(19) United States
(12) Patent Application Publication
(10) Pub. No.: US 2026/0297547 A1
(43) Pub. Date: Oct. 1, 2026

(54) METHODS AND COMPOSITIONS FOR MODIFYING GENETIC REPEATS

(71) Applicant: The General Hospital Corporation, Boston, MA (US)

(72) Inventor: Benjamin Kleinstiver, Boston, MA (US)

(21) Appl. No.: 19/478,664

(22) PCT Filed: Apr. 23, 2024
(86) PCT No.: PCT/US2024/025880

§ 371 Date: Oct. 24, 2025

Related U.S. Application Data
(60) Provisional application No. 63/497,943, filed on Apr. 24, 2023.

(51) Int. Cl. C12N 9/22; A61K 31/7105; A61K 38/46; C12N 15/11; C12N 15/90

(52) U.S. Cl. CPC C12N 9/226; A61K 31/7105; A61K 38/465; C12N 15/11; C12N 15/907; C12N 2310/20; C12N 2320/32

(57) ABSTRACT

Methods and compositions for contracting an expansion of CAG trinucleotide repeats in a huntingtin (HTT) gene in a cell, comprising Cas-based enzymes and a guide RNA (gRNA) that directs the Cas-based enzyme to the CAG nucleotide repeat.

### CLAIM OF PRIORITY

This application is a § 371 U.S. National Phase Application of International Patent Application No. PCT/US2024/025880, filed on Apr. 23, 2024, which claims the benefit of U.S. Provisional Patent Application Ser. No. 63/497,943, filed on Apr. 24, 2023. The entire contents of the foregoing are hereby incorporated by reference.

### SEQUENCE LISTING

This application contains a Sequence Listing that has been submitted electronically as an XML file named “29539-0744WO1_SL.xml.” The XML file, created on Jul. 29, 2024, is 161,912 bytes in size. The material in the XML file is hereby incorporated by reference in its entirety.

### TECHNICAL FIELD

Methods and compositions for contracting an expansion of nucleotide repeats, e.g., CAG trinucleotide repeats in a huntingtin (HTT) gene, in a cell, comprising various Cas-based enzymes and guide RNAs (gRNAs) that directs the Cas-based enzymes to the CAG nucleotide repeat.

### BACKGROUND

Various diseases, including but not limited to neurological and motor diseases, can result from expanded nucleotide repeat sequences within human genes. These expanded sequences can lead to a variety of consequences including decreased gene expression, the production of dominant negative proteins, RNA transcripts or protein aggregates, and other molecular pathologies. One prominent and well-studied example of a trinucleotide repeat expansion is the CAG nucleotide repeat found within the first exon of the Huntingtin gene (HTT). Translation of a mutant HTT allele (mHTT) encoding more than 40 CAG repeats results in the expression of a dominant negative mHTT protein implicated in Huntington's Disease (HD) pathogenesis.

Generally, onset of HD is inversely correlated with CAG-repeat length, whereas poor prognosis is strongly associated with an increased number of expanded triplets.

### SUMMARY

Because there is currently no cure for HD, one potential strategy to abrogate the neuromuscular and cognitive decline of HD individuals could be to selectively eliminate the mHTT protein by targeting and contracting the mHTT allele to a shorter non-pathogenic repeat length. Towards this goal, here we developed genome editing approaches that directly perturb the genetic cause of HD by targeting and permanently modifying the CAG-repeat length. To do so, we leveraged the distinct properties of different CRISPR systems, including engineered variants of Cas9 and Cas12a nucleases, nickases, and base editors that are more amenable to targeting and contracting repeats. We developed Cas9 nucleases and nickases capable of targeting the HTT CAG-repeat, leveraged distinctive properties of Cas12a nucleases to contract the HTT CAG-repeat, and explored the feasibility of base editors and engineered repeat editor enzymes to modify or reduce the length of the HTT CAG-repeat. Collectively, we thoroughly examined the potential of CRISPR methods and technologies to eliminate the HD-causative trinucleotide repeat, with the goal of identifying pre-clinical strategies towards a permanent genetic cure for HD patients. We envision that this approach should be extensible to other genetics repeats of various sequence compositions and their associated diseases.

Provided herein are methods of contracting nucleotide repeat expansion, e.g., an expansion of CAG trinucleotide repeats in a huntingtin (HTT) gene, in a cell. The methods comprise contacting the cell with or expressing in the cell a Cas-based enzyme and a guide RNA (gRNA) that directs the Cas-based enzyme to the CAG nucleotide repeat, preferably wherein the gRNA binds to an exon/repeat border, in an amount sufficient to reduce the number of nucleotide repeats in the cell.

Also provided herein are methods of treating a subject who has a condition associated with nucleotide repeat expansion, e.g., CAG nucleotide repeat expansion in a HTT gene. The methods comprise administering to the subject a therapeutically effective amount of a Cas-based enzyme and a guide RNA that directs the Cas-based enzyme to the nucleotide repeat, preferably wherein the gRNA binds to an exon/repeat border, in an amount sufficient to reduce the number of nucleotide repeats in the cell.

In some embodiments, the Cas-based enzyme and gRNA are administered to the CNS, e.g., brain or spinal cord of the subject (e.g., via ICV, cisternae magna, or intrathecal administration), or administered systemically to the subject.

In some embodiments, the Cas-based enzyme comprises Cas9, optionally SpG, or SpRY SaCas9, Nme2Cas9, or CjeCas9; or Cas12a, optionally enAsCas12a. In some embodiments, the methods use an IscB enzyme in place of a Cas-based enzyme.

In some embodiments, the Cas-based enzyme is:
(i) a Cas9 nickase, optionally a SpG nickase, or SpRY nickase;
(ii) a Cas12a nickase, optionally a enAsCas12a nickase;
(iii) a Cas9- or Cas12a-Base editor (BE) as described herein comprising a nicking or catalytically inactive Cas9, SpG, SpRY, Cas12a, or enAsCas12a and a UGI, or
(iv) a Cas9- or Cas12a-Repeat editor as described herein comprising a nicking or catalytically inactive Cas9, SpG, SpRY, Cas12a, or enAsCas 12a and lacking a UGI.

In some embodiments, the gRNA is listed in Table 1 or 2. In some embodiments, the 5′ end of the spacer may be extended or substituted to include alternate nucleotide compositions to modify transcription from polIII promoters. The spacer sequence can be, e.g., 20 nucleotides (nt) with a matched 5′ guanine (G), 20 nt with a mismatched 5′G, 20 nt with matched or mismatched alternate nts, 21 nt with an extended matched or mismatched 5′G or other nts, or 22 nt with extended matched or mismatched nts optionally including a 5′G. In some embodiments, the Cas12a target sites have exemplary 23 nt spacer sequences; the spacer sequences for the Cas12a crRNAs may be truncated to 22, 21, 20, 19, 18, or 17 nt (by removing bases from the 3′ PAM distal end of the spacer).

In some embodiments, the Cas-based enzyme and gRNA are as listed in Table 4.

In some embodiments, the Cas-based enzyme and gRNA are administered in an expression vector, e.g., a plasmid or viral vector; are administered as mRNA; or are administered as RNPs.

In some embodiments, the number of CAG repeats is reduced to below 40 or below 26.

Additionally provided herein are compositions comprising a Cas-based enzyme and gRNA as described herein, e.g., as listed in Table 4, and nucleic acids encoding a Cas-based enzyme and gRNA as described herein, e.g., as listed in Table 4. In some embodiments, the Cas-based enzyme is a repeat editor as described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Repeat expansion disorders and Huntington's Disease. (A) Trinucleotide repeat sequences located within various locations of genes can result in pathogenic effects when the repeat length exceeds a tolerable threshold. (B) Schematic of the first exon of the Huntingtin gene. Expansion of the CAG-repeat in exon 1, which encodes a poly-glutamine (Q) tract, can lead to Huntington's Disease. Other sequence features near the CAG-repeat confound targeting, including repetitive sequences and high DNA GC-content. (C) Reduction of the CAG-repeat through genome editing may generate alleles with a reduced number of CAG repeats. Repeated cycles of CAG-targeting and target site reconstitution via endogenous DNA repair pathways can lead to progressive shortening of the CAG-repeat. FIG. 1C discloses SEQ ID NOS 155-156, respectively, in order of appearance.

FIGS. 2A-E. Genome editing with CRISPR enzymes. (A) Endogenous DNA repair pathways engage nuclease-induced double-strand breaks in DNA, resulting in different edit outcomes. NHEJ, non-homologous end-joining; MMEJ, micro-homology mediated end-joining; HDR, homology-directed repair. (B) Schematics of CRISPR-Cas9 and -Cas12a nucleases (top panel). Both enzymes must pair with a guide RNA (gRNA) to be able to scan the genome for the presence of protospacer-adjacent motifs (PAMs), the first critical step of target site recognition by CRISPR nucleases. Whereas Cas9 generates a blunt DSB in the PAM-proximal region of its target site (a region of high specificity, or intolerance of nucleotide mismatches), Cas12a leaves a staggered DSB with a 4-6 nt 5′ overhang in the very PAM distal end of its target site (where Cas12a has weak specificity, or an inability to discriminate sequence differences). (C, D) CRISPR enzymes have been adapted for other genome editing applications, including for epigenome editing (C) or base editing (D). The fusion of heterologous effector domains to either Cas9 or Cas12a enables these enzymes to regulate gene transcription (C) or enzymatically generate single nucleotide edits at defined sites (D). (E) PAM preferences of exemplary CRISPR-Cas enzymes, including wild-type or engineered SpCas9, SaCas9, and AsCas12a nucleases (for the engineered enzymes, see references^{31,32,40,41}).

FIGS. 3A-B. Targeting the HTT CAG-repeat with Cas9 nucleases and nickases. (A) Schematic of HTT exon 1 showing putative target sites for SpCas9 PAM variants in regions that flank the CAG-repeat (poly-Q region). Sites directly embedded within the CAG repeat will be examined with caution due to potential off-target specificity issues. (B) Experimental workflow for assessing the abilities of SpCas9 variant nucleases and nickases to contract the CAG-repeat. Genomic DNA from samples will be extracted at several different times following transfection to assess and compare the kinetics of CAG-repeat contraction for nucleases and nickases. Reduction in CAG repeat number will be assessed by PCR and capillary electrophoresis, with lead candidate samples analyzed by next-generation sequencing (NGS).

FIGS. 4A-B. Contraction of the CAG-repeat using an enhanced AsCas12a variant. (A) Target sites for broad PAM compatibility enAsCas12a variant in regions of HTT exon 1 that flank the CAG-repeat (poly-Q region). Sites directly embedded within the CAG repeat will be examined with caution due to potential off-target specificity issues. (B) Schematic of enAsCas12a-mediated contraction of the CAG-repeat. The DNA nicking sites of enAsCas12a occur in a region of its target site that has poor specificity (left panel), a potential advantage for initiating a targeting/repair/re-targeting cascade that can fully contract the CAG-repeat (right panel). FIG. 4B discloses SEQ ID NOS 157-158, respectively, in order of appearance.

FIGS. 5A-E. Targeted contraction of the HTT CAG-repeat using CRISPR repeat editor technologies. (A) Schematic of how repeat editor (REd) technologies can potentially be used to contract the CAG repeat. Briefly, the REd-driven deamination event will initiate base-excision repair (BER), leading to strand resection of the deaminated CAG-containing DNA strand. The complementary strand is postulated to form a hairpin during DNA repair, leading to exclusion of CAG repeats during repair of the resected strand. Subsequent re-targeting of the CAG repeat with REds will repeat the cycle to reduce the trinucleotide repeat to sub-pathogenic levels. (B) Diagram of a canonical SpCas9-BE; a deaminase domain and a uracil glycosylate inhibitor (UGI) domain are fused to a nickase version of SpCas9 (nCas9). This complex initiates deamination events on the solvent exposed non-target strand, made possible by the stability of the target strand DNA/gRNA R-loop. (C) Examples of commonly used cytosine-to-thymine (C-to-T) and adenine-to-guanine (A-to-G) BEs (CBEs and ABEs, respectively), and their respective ‘editing windows’. nCas9, nickase version of SpCas9; dCas12a, catalytically inactive version of Cas12a. (D) Detailed molecular architectures of prototypical CBEs and ABEs. (E) Components of CBEs and ABEs that will be varied in the current study to optimize REd/BE technologies for CAG-repeat contraction. FIG. 5A discloses SEQ ID NOS 159-162, respectively, in order of appearance.

FIGS. 6A-E. HTT CAG repeat targeting with WT SpCas9 nuclease. (A) Diagram of HTT exon 1, illustrating gRNAs that target sites with NGG PAMs (arrow annotations) and also the poly-Q (CAG) and poly-P regions. (B) Heatmap showing the percentage of base deletion across the target amplicon, as judged by NGS. High levels of editing indicate deletion of that base in a large fraction of sequenced alleles. The CAG poly-Q repeat begins at base 66 and ends at base 119; the poly-P repeat begins at base 120 and ends at base 152; mean shown for N=3 biological replicates. (C) Graph of the total editing observed in transfections using WT SpCas9 nuclease and various gRNAs. N=3, mean and SEM shown. (D) Average allele length resulting from transfections using WT SpCas9 nuclease and various gRNAs. Shorter alleles are evidence of CAG repeat contraction. The dashed line indicates the length of the reference allele. N=3, mean and SEM shown. (E) Proportion of reads that are in-frame or that are out-of-frame. FIG. 6A discloses SEQ ID NOS 163-164, respectively, in order of appearance.

FIGS. 7A-E. HTT CAG repeat targeting with SpG nuclease. (A) Diagram of HTT exon 1, illustrating gRNAs that target sites with NGN PAMs (arrow annotations) and also the poly-Q (CAG) and poly-P regions. (B) Heatmap showing the percentage of base deletion across the target amplicon, as judged by NGS. High levels of editing indicate deletion of that base in a large fraction of sequenced alleles. The CAG poly-Q repeat begins at base 66 and ends at base 119; the poly-P repeat begins at base 120 and ends at base 152; mean shown for N=3 biological replicates. (C) Graph of the total editing observed in transfections using SpG nuclease and various gRNAs. N=3, mean and SEM shown. (D) Average allele length resulting from transfections using SpG nuclease and various gRNAs. Shorter alleles are evidence of CAG repeat contraction. The dashed line indicates the length of the reference allele. N=3, mean and SEM shown. (E) Proportion of reads that are in-frame or that are out-of-frame. FIG. 7A discloses SEQ ID NOS 165-166, respectively, in order of appearance.

FIGS. 8A-E. HTT CAG repeat targeting with SpRY nuclease. (A) Diagram of HTT exon 1, illustrating gRNAs that target sites with NRN PAMs (arrow annotations) and also the poly-Q (CAG) and poly-P regions. (B) Heatmap showing the percentage of base deletion across the target amplicon, as judged by NGS. High levels of editing indicate deletion of that base in a large fraction of sequenced alleles. The CAG poly-Q repeat begins at base 66 and ends at base 119; the poly-P repeat begins at base 120 and ends at base 152; mean shown for N=3 biological replicates. (C) Graph of the total editing observed in transfections using SpRY nuclease and various gRNAs. N=3, mean and SEM shown. (D) Average allele length resulting from transfections using SpRY nuclease and various gRNAs. Shorter alleles are evidence of CAG repeat contraction. The dashed line indicates the length of the reference allele. N=3, mean and SEM shown. (E) Proportion of reads that are in-frame or that are out-of-frame. FIG. 8A discloses SEQ ID NOS 167 and 164, respectively, in order of appearance.

FIGS. 9A-I. HTT CAG repeat targeting with various SpCas9-based nickases. (A) Diagram of HTT exon 1, illustrating gRNAs that target sites with NGG PAMs (arrow annotations) and also the poly-Q (CAG) and poly-P regions. (B) Heatmap showing the percentage of base deletion across the target amplicon with SpCas9-D10A nickase, as judged by NGS. High levels of editing indicate deletion of that base in a large fraction of sequenced alleles. The CAG poly-Q repeat begins at base 66 and ends at base 119; the poly-P repeat begins at base 120 and ends at base 152. (C) Graph of the total editing observed in transfections using SpCas9-D10A nickase and various gRNAs. N=3, mean and SEM shown. (D) Heatmap showing the percentage of base deletion across the target amplicon with SpCas9-H840A nickase. (E) Graph of the total editing observed in transfections using SpCas9-H840A nickase and various gRNAs. N=3, mean and SEM shown. (F-I) Heatmaps showing the percentage of base deletion across the target amplicon with SpG-D10A nickase (F), SpG-H840A nickase (G), SpRY-D10A nickase (H), and SpRY-H810A nickase (I). Percent deletions were evaluated by NGS; mean shown for N=3 biological replicates. FIG. 9A discloses SEQ ID NOS 163-164, respectively, in order of appearance.

FIGS. 10A-B. HTT CAG repeat targeting and editing using enAsCas12a. (A) schematic of the first exon of HTT with enAsCas12a target sites shown. (B) editing efficiency using enAsCas12a and 8 different crRNAs targeted within or near the CAG repeat. Top panel: heatmap showing editing efficiency per crRNA (row) as a function of each base pair in the NGS amplicon; mean shown for N=3 biological replicates. Bottom left panel: total editing efficiency of each allele, quantifying any edit (insertion, deletion, or substitution). Bottom right panel: average allele length in a given replicate transfection per crRNA (dashed line indicates the length of the unmodified amplicon). All data was analyzed using a modified version of CRISPResso2; Mean, SD, and individual data points shown for n=3 independent biological replicate experiments. FIG. 10A discloses SEQ ID NOS 168-169, respectively, in order of appearance.

FIG. 11. Schematic of HTT exon 1. The amplicon used for base editor experiments is shown with the CAG repeat (poly-glutamine; poly-Q), poly-proline (poly-P), and gRNA target sites shown. Within the PCR amplicon used for next-generation sequencing (NGS) in the following FIGS. 12 and 13, the CAG/poly-Q repeat begins at base 53 and ends at base 109, and the poly-P repeat begins at base 110 and ends at base 136 (using primers oHES163 and oHES168 to amplify the target region). Figure discloses SEQ ID NOS 170-171, respectively, in order of appearance.

FIGS. 12A-F. HTT CAG repeat targeting with SpG base editors. (A) Heatmap showing the percentage of base deletion across the target amplicon in experiments using SpG cytosine base editors (SpG-CBEs) bearing different cytosine deaminase domains with gRNA HES264 (gRNA spacer sequence=GCAGCAGCAGCAGCAGCAGC (SEQ ID NO: 3); Table 1), as judged by NGS; mean shown for N=2 biological replicates. High levels of editing indicate deletion of that base in a large fraction of sequenced alleles. The CAG poly-Q repeat begins at base 53 and ends at base 109; the poly-P repeat begins at base 110 and ends at base 136. (B) Average allele length resulting from transfections using SpG-CBEs and gRNA HES264 (using the data from 12A). Shorter alleles are evidence of CAG repeat contraction. The dashed line indicates the length of the reference allele. N=2, mean, SEM, and individual datapoints shown. (C) Proportion of reads that are in-frame or that are out-of-frame (using the data from 12A; N=2, mean, SEM, and individual datapoints shown). (D) Heatmap showing the percentage of base deletion across the target amplicon in experiments using SpG cytosine base editors (SpG-CBEs) bearing different cytosine deaminase domains with gRNA HES283 (gRNA spacer sequence=GTGCTGCTGGAAGGACTTGAG (SEQ ID NO: 4); Table 1), as judged by NGS; mean shown for N=2 biological replicates. High levels of editing indicate deletion of that base in a large fraction of sequenced alleles. The CAG poly-Q repeat begins at base 53 and ends at base 109; the poly-P repeat begins at base 110 and ends at base 136. (E) Average allele length resulting from transfections using SpG-CBEs and gRNA HES283 (using the data from 12D). Shorter alleles are evidence of CAG repeat contraction. The dashed line indicates the length of the reference allele. N=2, mean, SEM, and individual datapoints shown. (F) Proportion of reads that are in-frame or that are out-of-frame (using the data from 12D; N=2, mean, SEM, and individual datapoints shown). All BEs are nickase Cas9 enzymes (D10A).

FIGS. 13A-I. HTT CAG repeat targeting with SpRY base editors. (A) Heatmap showing the percentage of base deletion across the target amplicon in experiments using SpRY cytosine base editors (SpRY-CBEs) bearing different cytosine deaminase domains with gRNA HES253 (gRNA spacer sequence=GAGCAGCAGCAGCAGCAGCAG (SEQ ID NO: 5); Table 1), as judged by NGS; mean shown for N=2 biological replicates. High levels of editing indicate deletion of that base in a large fraction of sequenced alleles. The CAG poly-Q repeat begins at base 53 and ends at base 109; the poly-P repeat begins at base 110 and ends at base 136. (B) Average allele length resulting from transfections using SpRY-CBEs and gRNA HES253 (using the data from 13A). Shorter alleles are evidence of CAG repeat contraction. The dashed line indicates the length of the reference allele. N=2, mean, SEM, and individual datapoints shown. (C) Proportion of reads that are in-frame or that are out-of-frame (using the data from 13A; N=2, mean, SEM, and individual datapoints shown). (D) Heatmap showing the percentage of base deletion across the target amplicon in experiments using SpRY cytosine base editors (SpRY-CBEs) bearing different cytosine deaminase domains with gRNA HES256 (gRNA spacer sequence=GTGCTGCTGCTGGAAGGACTT (SEQ ID NO: 6); Table 1), as judged by NGS; mean shown for N=2 biological replicates. High levels of editing indicate deletion of that base in a large fraction of sequenced alleles. The CAG poly-Q repeat begins at base 53 and ends at base 109; the poly-P repeat begins at base 110 and ends at base 136. (E) Average allele length resulting from transfections using SpRY-CBEs and gRNA HES256 (using the data from 13D). Shorter alleles are evidence of CAG repeat contraction. The dashed line indicates the length of the reference allele. N=2, mean, SEM, and individual datapoints shown. (F) Proportion of reads that are in-frame or that are out-of-frame (using the data from 13D; N=2, mean, SEM, and individual datapoints shown). (G) Heatmap showing the percentage of base deletion across the target amplicon in experiments using SpRY cytosine base editors (SpRY-CBEs) bearing different cytosine deaminase domains with gRNA HES254 (gRNA spacer sequence=GCGCCGCCGCCGCCGCCTCCT (SEQ ID NO: 7); Table 1), as judged by NGS; mean shown for N=2 biological replicates. High levels of editing indicate deletion of that base in a large fraction of sequenced alleles. The CAG poly-Q repeat begins at base 53 and ends at base 109; the poly-P repeat begins at base 110 and ends at base 136. (H) Average allele length resulting from transfections using SpRY-CBEs and gRNA HES254 (using the data from 13G). Shorter alleles are evidence of CAG repeat contraction. The dashed line indicates the length of the reference allele. N=2, mean, SEM, and individual datapoints shown. (I) Proportion of reads that are in-frame or that are out-of-frame (using the data from 13G; N=2, mean, SEM, and individual datapoints shown). All BEs are nickase Cas9 enzymes (D10A).

FIG. 14. Schematic of HTT exon 1. The amplicon used for base editor (BE) and repeat editor (REd) experiments is shown with the CAG repeat (poly-glutamine; poly-Q), poly-proline (poly-P), and gRNA target sites shown. Within the PCR amplicon used for next-generation sequencing (NGS) in the following FIGS. 15 and 16, the CAG/poly-Q repeat begins at base 66 and ends at base 119, and the poly-P repeat begins at base 120 and ends at base 152. Figure discloses SEQ ID NOS 172-173, respectively, in order of appearance.

FIGS. 15A-L. HTT CAG repeat targeting with SpG base editors and repeat editors. (A) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), with gRNA HES264 (gRNA spacer sequence=GCAGCAGCAGCAGCAGCAGC (SEQ ID NO: 3); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (B) Average allele length resulting from transfections using SpG-CBEs or -REds and gRNA HES264 (using the data from 15A). Shorter alleles are evidence of CAG repeat contraction. The dashed line indicates the length of the reference allele. N=3, mean, SEM, and individual datapoints shown. (C) Proportion of reads that are in-frame or that are out-of-frame (using the data from 15A; N=3, mean, SEM, and individual datapoints shown). (D) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), with gRNA HES265 (gRNA spacer sequence=GCTGCTGCTGCTGCTGCTGC (SEQ ID NO: 8); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (E) Average allele length resulting from transfections using SpG-CBEs or -REds and gRNA HES265 (using the data from 15D). Shorter alleles are evidence of CAG repeat contraction. The dashed line indicates the length of the reference allele. N=3, mean, SEM, and individual datapoints shown. (F) Proportion of reads that are in-frame or that are out-of-frame (using the data from 15D; N=3, mean, SEM, and individual datapoints shown). (G) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), with gRNA HES284 (gRNA spacer sequence=GCTGCTGCTGCTGGAAGGACT (SEQ ID NO: 9); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (H) Average allele length resulting from transfections using SpG-CBEs or -REds and gRNA HES284 (using the data from 15G). Shorter alleles are evidence of CAG repeat contraction. The dashed line indicates the length of the reference allele. N=3, mean, SEM, and individual datapoints shown. (I) Proportion of reads that are in-frame or that are out-of-frame (using the data from 15G; N=3, mean, SEM, and individual datapoints shown). (J) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), with gRNA RTW5 (gRNA spacer sequence=GCTGCTGCTGCTGCTGCTGGA (SEQ ID NO: 10); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (K) Average allele length resulting from transfections using SpG-CBEs or -REds and gRNA RTW5 (using the data from 15J). Shorter alleles are evidence of CAG repeat contraction. The dashed line indicates the length of the reference allele. N=3, mean, SEM, and individual datapoints shown. (L) Proportion of reads that are in-frame or that are out-of-frame (using the data from 15J; N=3, mean, SEM, and individual datapoints shown). All BEs or REds are nickase Cas9 enzymes (D10A).

FIGS. 16A-R. HTT CAG repeat targeting with SpG base editors and modified repeat editors. (A) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), and with or without an additional nuclease inactivating H840A mutation (dead or dCas9), all with gRNA HES264 (gRNA spacer sequence=GCAGCAGCAGCAGCAGCAGC (SEQ ID NO: 3); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (B) Average allele length resulting from transfections using SpG-CBEs or -REds and gRNA HES264 (using the data from 16A). Shorter alleles are evidence of CAG repeat contraction. The dashed line indicates the length of the reference allele. N=3, mean, SEM, and individual datapoints shown. (C) Proportion of reads that are in-frame or that are out-of-frame (using the data from 16A; N=3, mean, SEM, and individual datapoints shown). (D) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), and with or without an additional nuclease inactivating H840A mutation (dead or dCas9), all with gRNA HES265 (gRNA spacer sequence=GCTGCTGCTGCTGCTGCTGC (SEQ ID NO: 8); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (E) Average allele length resulting from transfections using SpG-CBEs or -REds and gRNA HES265 (using the data from 16D). Shorter alleles are evidence of CAG repeat contraction. The dashed line indicates the length of the reference allele. N=3, mean, SEM, and individual datapoints shown. (F) Proportion of reads that are in-frame or that are out-of-frame (using the data from 16D; N=3, mean, SEM, and individual datapoints shown). (G) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), and with or without an additional nuclease inactivating H840A mutation (dead or dCas9), all with gRNA HES284 (gRNA spacer sequence=GCTGCTGCTGCTGGAAGGACT (SEQ ID NO: 9); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (H) Average allele length resulting from transfections using SpG-CBEs or -REds and gRNA HES284 (using the data from 16G). Shorter alleles are evidence of CAG repeat contraction. The dashed line indicates the length of the reference allele. N=3, mean, SEM, and individual datapoints shown. (I) Proportion of reads that are in-frame or that are out-of-frame (using the data from 16G; N=3, mean, SEM, and individual datapoints shown). (J) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), and with or without an additional nuclease inactivating H840A mutation (dead or dCas9), all with gRNA RTW5 (gRNA spacer sequence=GCTGCTGCTGCTGCTGCTGGA (SEQ ID NO: 10); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (K) Average allele length resulting from transfections using SpG-CBEs or -REds and gRNA RTW5 (using the data from 16J). Shorter alleles are evidence of CAG repeat contraction. The dashed line indicates the length of the reference allele. N=3, mean, SEM, and individual datapoints shown. (L) Proportion of reads that are in-frame or that are out-of-frame (using the data from 16J; N=3, mean, SEM, and individual datapoints shown). (M) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), and with or without an additional nuclease inactivating H840A mutation (dead or dCas9), all with gRNA HES283 (gRNA spacer sequence=GTGCTGCTGGAAGGACTTGAG (SEQ ID NO: 4); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (N) Average allele length resulting from transfections using SpG-CBEs or -REds and gRNA HES283 (using the data from 16M). Shorter alleles are evidence of CAG repeat contraction. The dashed line indicates the length of the reference allele. N=3, mean, SEM, and individual datapoints shown. (O) Proportion of reads that are in-frame or that are out-of-frame (using the data from 16M; N=3, mean, SEM, and individual datapoints shown). (P) Heatmap showing the percentage of base deletion across the target amplicon in experiments using SpG-based cytosine base editors (SpG-CBEs) bearing different cytosine deaminase domains, and engineered SpG-based repeat editors (SpG-REds) bearing different cytosine deaminase domains without the uracil glycosylase inhibitor (UGI), and with or without an additional nuclease inactivating H840A mutation (dead or dCas9), all with gRNA HES285 (gRNA spacer sequence=GTGCTGCTGCTGCTGCTGGAA (SEQ ID NO: 11); Table 1), as judged by NGS; mean shown for N=3 biological replicates. High levels of editing indicate deletion of that base in a large fraction of sequenced alleles. The CAG poly-Q repeat begins at base 66 and ends at base 119; the poly-P repeat begins at base 120 and ends at base 152. (Q) Average allele length resulting from transfections using SpG-CBEs or -REds and gRNA HES285 (using the data from 16P). Shorter alleles are evidence of CAG repeat contraction. The dashed line indicates the length of the reference allele. N=3, mean, SEM, and individual datapoints shown. (R) Proportion of reads that are in-frame or that are out-of-frame (using the data from 16P; N=3, mean, SEM, and individual datapoints shown). BEs or REds are nickase Cas9 enzymes (D10A) unless otherwise indicated that they are catalytically inactive (or dead; dCas9, which are then harboring an additional H840A mutation).

FIGS. 17A-I. HTT exon 1 CAG repeat targeting and editing over time. (A-I) Heatmaps showing the percentage of next-generation sequencing (NGS) reads with deletions at individual bases across the target amplicon. Experiments were performed by transfecting plasmids expressing enzymes and gRNAs, followed by extraction of genomic DNA over various timepoints (rows in the heatmaps) and NGS to determine editing and deletions within HTT exon 1. Enzymes used in this experiment include SpCas9 nuclease (A-C), SpG nuclease (D and E), enAsCas12a nuclease (F and G), and repeat editors (REds) comprising CDA1-dSpG-[no-UGI] (H) or CDA1-nSpG-[no-UGI] (I). The Cas9 gRNAs are listed in Table 1, Cas12a crRNAs in Table 2, and enzymes in Table 3. Figure discloses SEQ ID NOS 39, 17, 51, 62, 68, 142, 135, 74 and 17, respectively, in order of appearance.

### DETAILED DESCRIPTION

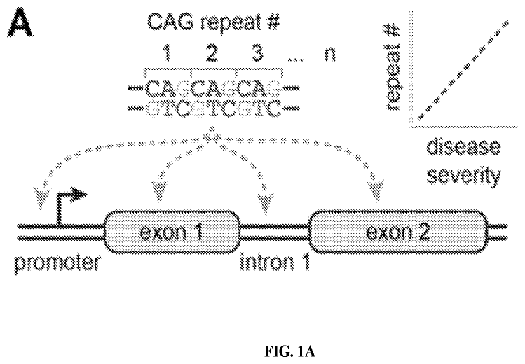

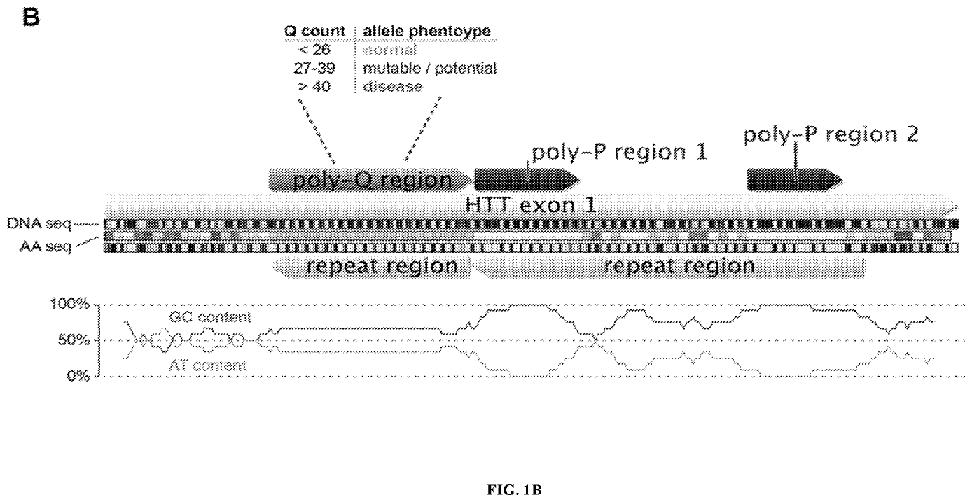

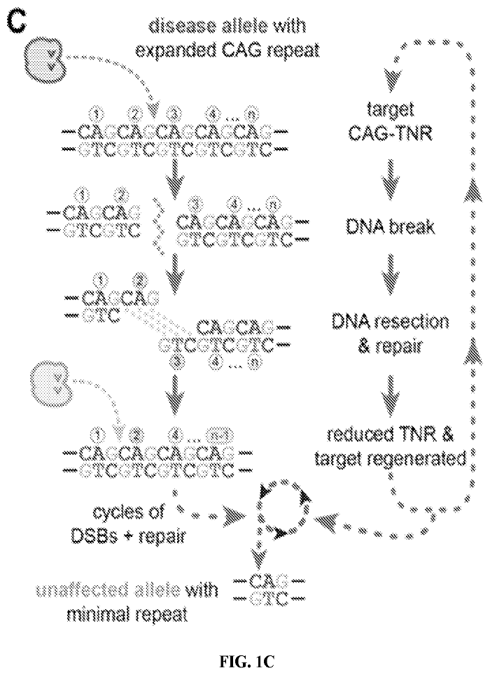

When unstable and repetitive sequences in the human genome are inadvertently expanded to supra-physiological lengths, gene dysregulation can often lead to disease pathogenesis. Consequently, more than two dozen diseases are suspected to be the result of extended and repetitive DNA sequences¹. One such class of disorders, characterized by trinucleotide repeat expansions, are thought to occur as the result of errors during DNA replication or transcription, including polymerase slippage, stalled or blocked replication forks, or spontaneous DNA breaks²⁻⁵. Trinucleotide repeat sequences have been identified near or within promoters, exons, introns, and 5′ or 3′ UTRs of human genes, with the length of the repeat often correlating with severity of the disease⁵ (FIG. 1a). Huntington's disease (HD) is one prominent example of an autosomal dominant trinucleotide repeat disorder caused by the expansion of a CAG sequence within the first exon of the Huntingtin gene (HTT; FIG. 1b). Genome sequencing of HD-affected and unaffected individuals has revealed that, in general, mutant HTT alleles (mHTT) with CAG expansions longer than 40 units are pathogenic^{6,7}. The accumulation of mHTT proteins (that harbor an extended poly-glutamine (polyQ) peptide sequence, a result of the CAG-repeat) is thought to lead to aggregation and atypical interactions with other proteins, causing neuronal cell death that results in movement and mental impairment characteristic of HD^{8,9}. While clinical strategies are available to manage symptoms and prolong HD patient life, no therapeutic approaches currently exist to directly correct the underlying genetic cause of HD. As a dominant negative disease, HD symptoms occur when a single mHTT allele is present; thus, one potential therapeutic approach to treat HD would be to exploit genome editing technologies to reduce the CAG-repeat on the disease-causing allele to a sub-pathogenic number of repeats (FIG. 1c).

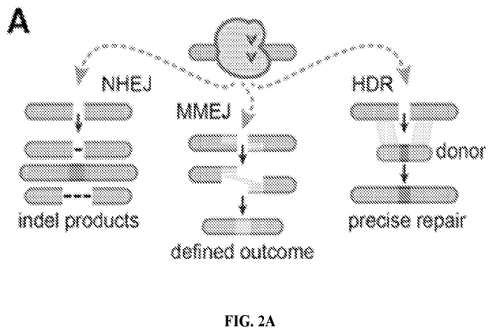

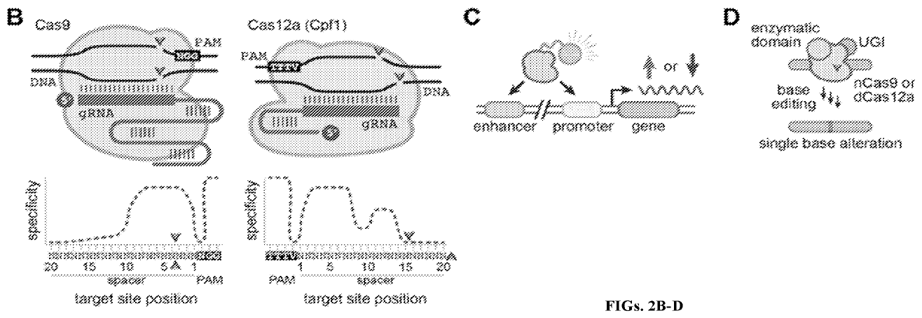

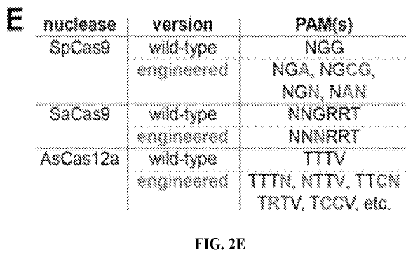

Recent advances in genome editing technologies have revolutionized a number of scientific disciplines as they now enable the efficient alteration of genomic sequence or the temporal control of gene expression¹⁰⁻¹³ Engineered nucleases can be programmed to induce double-stand breaks (DSBs) in DNA at specified sites in the genome, with heritable genetic changes resulting when endogenous DNA repair pathways are engaged to correct the break. Non-homologous end-joining (NHEJ) repair can introduce variable-length insertion or deletion mutations (indels), micro-homology mediated repair (MMEJ) can result in sequence-defined repair events, and homology directed repair (HDR) can generate precise, user-specified changes when an exogenous homologous repair template is provided in trans^{12,14-16} (FIG. 2a). Genome editing platforms that have been engineered to introduce site-specific DSBs in cells include homing endonucleases, zinc-finger nucleases (ZFNs), transcription activator-like effector nucleases (TALENs), and the more recently described monomeric clustered regularly interspaced short palindromic repeat (CRISPR) nucleases Cas9 and Cas12a^{10,17} (formerly named Cpf1; FIG. 2b). Because genome editing technologies enable the permanent manipulation of DNA sequences, they hold promise to treat the underlying genetics of repeat expansion diseases such as HD via the stable reduction of the repeat sequence⁴.

CRISPR nucleases have been adapted for genome editing and have been used widely for manipulating DNA sequences. Beyond the prototypical application of CRISPR nucleases to permanently edit DNA sequences, effector domains can be fused to catalytically inactivated nucleases to generate sequence-specific DNA-binding proteins capable of transcriptional regulation for epigenome editing^{18,19} (FIG. 2c), and other enzymatic functions including DNA deamination for base editing^{16,20-22} (FIG. 2d) or reverse transcriptase domains for prime editing²³. A major reason for the widespread implementation of CRISPR enzymes is ease of use. To initiate targeting of genomic sequences, most CRISPR nucleases require the reprogramming of a segment of a guide RNA (gRNA) to be complementarity to the intended target DNA²⁴⁻²⁶, making targeting new sites more straightforward compared to prior technologies. A second requirement for targeting is the presence of short protospacer-adjacent motif (PAM) recognized by the nuclease itself¹¹⁻¹³ (FIG. 2b). Once programmed with a gRNA, the nuclease/gRNA complex scans the genome for sites that encode a PAM. For the prototypical CRISPR-Cas9 nuclease from Streptococcus pyogenes (SpCas9), the PAM requirement of an NGG sequence restricts targeting to sites that encode this motif. Once the Cas9/gRNA complex recognizes the PAM, if the DNA sequence adjacent to the PAM is sufficiently complementary to the sequence of the gRNA, the nuclease can induce a DSB to initiate genome-editing events. Thus, PAM readout by CRISPR nucleases is the first critical step of target site recognition, which consequently restricts targeting to genomic loci that encode PAMs (and in many cases may limit targeting of certain DNA sequences) (FIG. 2e). Despite this and other limitations (areas that are the subject of active research²⁷), CRISPR nucleases hold tremendous promise for the correction of genetic diseases.

Here we explored the use of genome editing technologies to perturb genetic repeats, towards correcting the underlying genetic cause of repeat expansion diseases like HD. To do so, we explored novel approaches to induce in-frame contraction of the HTT CAG-repeat, including engineered CRISPR nucleases, nickases, and base editors capable of targeting near or straddling the repeat, avoiding unwanted genome-wide off-target edits that would occur when targeting solely within the CAG repeat (since CAG-repeats are distributed throughout the human genome). While recent proof-of-concept studies explored the feasibility of genome editing to treat HD²⁸⁻³⁰, the efficacy of each approach was limited by the inherent characteristics of naturally occurring CRISPR technologies⁴. One property of the widely used SpCas9 that has prevented its successful application for addressing different diseases is the lack of targetable PAMs near the site of the genetic mutation. The PAM restriction of CRISPR nucleases manifests clearly in the context of HD and the HTT-CAG locus, as there are no targetable NGG PAMs within the CAG-repeat. Additionally, there are other complex sequence features adjacent to the CAG-repeat that impose targeting constraints, including: (1) a relative paucity of canonical SpCas9 NGG PAMs adjacent to the CAG repeat, (2) the presence of two separate extended poly-proline repeat regions (poly-P1 and poly-P2), (3) high GC-content, and (4) relatively sparse unique sequence to target due to the repetitiveness of the locus (FIG. 1b). These complexities are further exacerbated by the genome-wide occurrence of CAG repeats, where the simultaneous targeting of the other non-HTT CAG repeats could cause catastrophic genome-scale consequences (due to translocations, cell toxicity, etc.). Using our engineered SpG and SpRY enzymes³¹, we can overcome both the targeting range and specificity drawbacks of prior genome editing studies. The overarching goal of this research is therefore to develop and utilize novel CRISPR-based genome editing methods and enzymes that can safely and efficiently address the CAG-repeat to return the mHTT locus to a sub-pathogenic length.

Because the internal and flanking sequence of the mHTT allele are nearly devoid of NGG PAMs recognized by SpCas9, targeting the CAG-repeat is limited when using conventional technologies. Towards relieving the targeting constraint, we previously engineering novel variants of SpCas9 that recognize alternate NGA and NGCG PAMs³² (FIG. 2e). In a subsequent study, we relaxed the more restrictive PAM of Staphylococcus aureus Cas9 (SaCas9) to quadruple its targeting range from NNGRRT to NNNRRT PAMs^{33,34} (FIG. 2e). Furthermore, we characterized the beneficial activities and specificities of CRISPR-Cas12a nucleases as genome editing reagents³⁵. Cas 12a nucleases offer potential advantages over Cas9 (FIG. 2b), including the abilities to: 1) target T-rich sequences³⁶, 2) induce DSBs at the PAM distal end of the spacer³⁵⁻³⁷, and 3) process multiple gRNAs out of a single RNA transcript³⁸⁻⁴⁰. We also recently described several engineered Acidaminococcus sp. Cas12a (AsCas12a) variants with expanded targeting ranges (FIG. 2e) and improved on-target activities⁴¹, leading to an enhanced AsCas12a (enAsCas12a) enzyme. Finally, we described the generation of novel SpCas9 variants of that can target NGN PAMs (now enabling targeting of NGC and NGT PAMs) and NRN>NYN PAMs, named SpG and SpRY, respecitvely³¹ (FIG. 2e). These SpG and SpRY variants permit high-density targeting at the border of, and within, the CAG-repeat.

Described herein are CRISPR-based gene-editing technologies and strategies for reducing the HD-causative CAG-repeat to sub-pathogenic levels. We utilized our comprehensive suite of genome editing technologies to contract the pathogenic repeat by generating DNA breaks, nicks, or deamination events within or near the CAG-repeat.

### Methods of Use

In some embodiments, the present methods and compositions can be used to contract Huntington's disease (HD)-associated CAG nucleotide expansions in a living cell or subject, or a cell or population of cells from a subject, who has HD associated with CAG nucleotide expansions, e.g., more than 26 or more than 40 CAG repeats in the HTT gene. Although exemplified on CAG repeats in HTT, the present methods can also be used in other nucleotide expansion diseases, e.g., as described in WO2022197857.

The present methods and compositions can be used to treat subjects who have Huntington's disease (HD). A diagnosis of HD can be made using methods known in the art. The cause of Huntington's disease was found to be a CAG expansion in exon 1 of the huntingtin gene (HTT). The disease protein contains a polyglutamine expansion in the N-terminal region of the Huntingtin protein (HTT) (Ellerby, L. M. (2019) Neurotherapeutics 16:924-927). Unaffected individuals may have roughly 6-29 CAG triplets in both alleles; yet, in HD patients, the disease allele may contain 36 to hundreds of CAG triplets. As the repeat number grows, the growing polyglutamine tract produces an abnormal HD gene product (called huntingtin) with increasingly aberrant properties that causes death of brain cells controlling movement (Budworth, H. and McMurray, C. T. (2013) Methods Mol Biol. 1010:3-17). In some embodiments, the methods and compositions described herein can be administered to a cell or subject having >30 repeats, e.g., 30-100 repeats, or >100 repeats. In some embodiments, the methods and compositions described herein methods can reduce levels of huntingtin.

In some embodiments, the subject has demonstrated signs of HD; in some embodiments, the subject has not yet demonstrated signs of HD. The methods can thus be used to ameliorate one or more symptoms of HD, e.g., to reduce severity of one or more symptoms; to reduce the likelihood that a subject will develop one or more symptoms of HD; or to slow progression or worsening of one or more symptoms of HD.

The methods include delivering to the cell or subject a CRISPR Cas protein (optionally in a base editor) and a guide RNA directing the Cas protein to the expansion sequence in the HTT gene.

The methods can include obtaining iPSC generated from differentiated somatic cells obtained from the subject; exposing the iPSC to a treatment described herein to contract (reduce the number of) nucleotide repeats; optionally promoting differentiation of the corrected cells, e.g., to neural precursor cells; and administering the cells to the subject, e.g., to the CNS (spinal cord or brain) of a subject, such as to the cortex, cerebellum, hypothalamus, substantia nigra, spinal cord, putamen, hippocampus, or other CNS regions (see, e.g., Duma et al., Molecular Biology Reports volume 46, pages 5257-5272 (2019); Schweitzer et al., N Engl J Med. 2020 May 14; 382 (20): 1926-1932; Kim et al., Alzheimers Dement (N Y). 2015 September; 1 (2): 95-102), or to one or more organs (e.g., liver, lung, heart, kidney, or gut). See, e.g., WO2022197857. Alternatively, the methods can include administering a composition as described herein to the subject, e.g., to the CNS of the subject (e.g., via ICV, cisternae magna, or intrathecal administration), to an organ (e.g., liver, lung, heart, kidney, or gut) or systemically.

### CRISPR/Cas Targeting of HTT Nucleotide Repeats

The present methods include using variants of Cas proteins, e.g., Cas9 or Cas12 proteins with altered PAM specificity, including nucleases, nickases, and base editors; in some embodiments, the Cas protein is not catalytically inactive (dCas) unless it is present in a base editor or repeat editor. These methods can be applied to other CRISPR-Cas proteins, including other Cas9 orthologs with various levels of basal activity (SaCas9, StlCas9, St3Cas9, NmeCas9, Nme2Cas9, CjeCas9, etc.), Cas12a orthologs, and other Cas3, Cas12, Cas13, and Cas14 proteins.

The Cas proteins can be incorporated into existing and widely used vectors, e.g., by simple site-directed mutagenesis, and can also be combined with other previously described improvements to the SpCas9 platform (e.g., truncated sgRNAs (Tsai et al., Nat Biotechnol 33, 187-197 (2015); Fu et al., Nat Biotechnol 32, 279-284 (2014)), nickase mutations (Mali et al., Nat Biotechnol 31, 833-838 (2013); Ran et al., Cell 154, 1380-1389 (2013)), dimeric FokI-dCas9 fusions (Guilinger et al., Nat Biotechnol 32, 577-582 (2014); Tsai et al., Nat Biotechnol 32, 569-576 (2014)); and high-fidelity variants (Kleinstiver et al. Nature 2016).

### SpCas9 Variants

In some embodiments, the present methods and compositions use Cas proteins comprising an SpCas9 variant. The SpCas9 wild type sequence is as follows:

(SEQ ID NO: 1)
MDKKYSIGLD IGTNSVGWAV ITDEYKVPSK KFKVLGNTDR HSIKKNLIGA LLFDSGETAE
ATRLKRTARR RYTRRKNRIC YLQEIFSNEM AKVDDSFFHR LEESFLVEED KKHERHPIFG
NIVDEVAYHE KYPTIYHLRK KLVDSTDKAD LRLIYLALAH MIKFRGHFLI EGDLNPDNSD
VDKLFIQLVQ TYNQLFEENP INASGVDAKA ILSARLSKSR RLENLIAQLP GEKKNGLFGN
LIALSLGLTP NFKSNFDLAE DAKLQLSKDT YDDDLDNLLA QIGDQYADLF LAAKNLSDAI
LLSDILRVNT EITKAPLSAS MIKRYDEHHQ DLTLLKALVR QQLPEKYKEI FFDQSKNGYA
GYIDGGASQE EFYKFIKPIL EKMDGTEELL VKLNREDLLR KQRTFDNGSI PHQIHLGELH
AILRRQEDFY PFLKDNREKI EKILTFRIPY YVGPLARGNS RFAWMTRKSE ETITPWNFEE
VVDKGASAQS FIERMTNFDK NLPNEKVLPK HSLLYEYFTV YNELTKVKYV TEGMRKPAFL
SGEQKKAIVD LLFKTNRKVT VKQLKEDYFK KIECFDSVEI SGVEDRFNAS LGTYHDLLKI
IKDKDFLDNE ENEDILEDIV LTLTLFEDRE MIEERLKTYA HLFDDKVMKQ LKRRRYTGWG
RLSRKLINGI RDKQSGKTIL DFLKSDGFAN RNFMQLIHDD SLTFKEDIQK AQVSGQGDSL
HEHIANLAGS PAIKKGILQT VKVVDELVKV MGRHKPENIV IEMARENQTT QKGQKNSRER
MKRIEEGIKE LGSQILKEHP VENTQLQNEK LYLYYLQNGR DMYVDQELDI NRLSDYDVDH
IVPQSFLKDD SIDNKVLTRS DKNRGKSDNV PSEEVVKKMK NYWRQLLNAK LITQRKFDNL
TKAERGGLSE LDKAGFIKRQ LVETRQITKH VAQILDSRMN TKYDENDKLI REVKVITLKS
KLVSDFRKDF QFYKVREINN YHHAHDAYLN AVVGTALIKK YPKLESEFVY GDYKVYDVRK
MIAKSEQEIG KATAKYFFYS NIMNFFKTEI TLANGEIRKR PLIETNGETG EIVWDKGRDF
ATVRKVLSMP QVNIVKKTEV QTGGFSKESI LPKRNSDKLI YGGFDSPTVA YGGFDSPTVA
YSVLVVAKVE KGKSKKLKSV KELLGITIME RSSFEKNPID FLEAKGYKEV KKDLIIKLPK
YSLFELENGR KRMLASAGEL QKGNELALPS KYVNFLYLAS HYEKLKGSPE DNEQKQLFVE
QHKHYLDEII EQISEFSKRV ILADANLDKV LSAYNKHRDK PIREQAENII HLFTLTNLGA
PAAFKYFDTT IDRKRYTSTK EVLDATLIHQ SITGLYETRI DLSQLGGD

In some embodiments, the Cas9/nCas9/dCas9 has altered PAM specificity, e.g., SpG (mutations at D1135L/S1136W/G1218K/E1219Q/R1335Q/T1337R, which targets NGN PAM sequences), or SpRY (D1135L/S1136W/G1218K/E1219Q/R1335Q/T1337R/L1111R/A1322R/A61R/N1317R/R1333P mutations, which targets almost all PAM sequences (NRN and to a lesser extent NYN PAMs) (Walton et al., Science 26 Mar. 2020: eaba8853; WO 2021151085). In some embodiments, the SpCas9 comprises a mutation at D1135E (NGG PAM); mutations at D1135V, R1335Q and T1337R (NGAN or NGNG PAM); mutations at D1135V, G1218R, R1335Q and T1337R (NGAN or NGNG PAM); mutations at D1135E, R1335Q and T1337R (NGAG PAM); mutations at D1135V, G1218R, R1335E and T1337R (NGCG PAM).

The SpCas9 proteins can include mutations at one of the following amino acid positions to reduce (creating a nickase) or destroy the nuclease activity of the Cas9: D10, E762, D839, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432). In some embodiments, the variant includes mutations at D10 or H840 (which creates a single-strand nickase, nCas9), or mutations at D10 and H840 (which abrogates nuclease activity; this mutant is known as dead Cas9 or dCas9).

### AsCas12a Variants

In some embodiments, the present methods and compositions use Cas proteins comprising an AsCas12a variant. The AsCpf1 wild type protein sequence is as follows:

(SEQ ID NO: 2)
MTQFEGFTNL YQVSKTLRFE LIPQGKTLKH IQEQGFIEED KARNDHYKEL KPIIDRIYKT
YADQCLQLVQ LDWENLSAAI DSYRKEKTEE TRNALIEEQA TYRNAIHDYF IGRTDNLTDA
INKRHAEIYK GLFKAELFNG KVLKQLGTVT TTEHENALLR SFDKFTTYFS GFYENRKNVF
SAEDISTAIP HRIVQDNFPK FKENCHIFTR LITAVPSLRE HFENVKKAIG IFVSTSIEEV
FSFPFYNQLL TQTQIDLYNQ LLGGISREAG TEKIKGLNEV LNLAIQKNDE TAHIIASLPH
RFIPLFKQIL SDRNTLSFIL EEFKSDEEVI QSFCKYKTLL RNENVLETAE ALFNELNSID
LTHIFISHKK LETISSALCD HWDTLRNALY ERRISELTGK ITKSAKEKVQ RSLKHEDINL
QEIISAAGKE LSEAFKQKTS EILSHAHAAL DQPLPTTLKK QEEKEILKSQ LDSLLGLYHL
LDWFAVDESN EVDPEFSARL TGIKLEMEPS LSFYNKARNY ATKKPYSVEK FKLNFQMPTL
ASGWDVNKEK NNGAILFVKN GLYYLGIMPK QKGRYKALSF EPTEKTSEGF DKMYYDYFPD
AAKMIPKCST QLKAVTAHFQ THTTPILLSN NFIEPLEITK EIYDLNNPEK EPKKFQTAYA
KKTGDQKGYR EALCKWIDFT RDFLSKYTKT TSIDLSSLRP SSQYKDLGEY YAELNPLLYH
ISFQRIAEKE IMDAVETGKL YLFQIYNKDF AKGHHGKPNL HTLYWTGLFS PENLAKTSIK
LNGQAELFYR PKSRMKRMAH RLGEKMLNKK LKDQKTPIPD TLYQELYDYV NHRLSHDLSD
EARALLPNVI TKEVSHEIIK DRRFTSDKFF FHVPITLNYQ AANSPSKFNQ RVNAYLKEHP
ETPIIGIDRG ERNLIYITVI DSTGKILEQR SLNTIQQFDY QKKLDNREKE RVAARQAWSV
VGTIKDLKQG YLSQVIHEIV DLMIHYQAVV VLENLNFGFK SKRTGIAEKA VYQQFEKMLI
DKLNCLVLKD YPAEKVGGVL NPYQLTDQFT SFAKMGTQSG FLFYVPAPYT SKIDPLTGFV
DPFVWKTIKN HESRKHFLEG FDFLHYDVKT GDFILHFKMN RNLSFQRGLP GFMPAWDIVF
EKNETQFDAK GTPFIAGKRI VPVIENHRFT GRYRDLYPAN ELIALLEEKG IVFRDGSNIL
PKLLENDDSH AIDTMVALIR SVLQMRNSNA ATGEDYINSP VRDLNGVCFD SRFQNPEWPM
DADANGAYHI ALKGQLLLNH LKESKDLKLQ NGISNQDWLA YIQELRN

The AsCpf1 variants described herein can include the amino acid sequence of SEQ ID NO: 2, e.g., at least comprising amino acids 1-1307 of SEQ ID NO:2, with mutations (i.e., replacement of the native amino acid with a different amino acid, e.g., alanine, glycine, or serine (except where the native amino acid is serine)), at one or more positions in Table 1, e.g., at the following positions: T167, S170, E174, T539, K548, N551, N552, M604, and/or K607 of SEQ ID NO: 2. In some embodiments, the AsCpf1 variants are at least 80%, e.g., at least 85%, 90%, or 95% identical to the amino acid sequence of SEQ ID NO:2, e.g., have differences at up to 5%, 10%, 15%, or 20% of the residues of SEQ ID NO:2 replaced, e.g., with conservative mutations, in addition to the mutations described herein. In some embodiments, the AsCpf1 comprises mutations at E174R, S542R, and K548R (enAsCpf1 or enAsCas12a). In some embodiments, catalytic activity-destroying mutations are made at D908 and/or E993, e.g., D908A and E993A, or R1226, e.g., R1226A. In preferred embodiments, the variant retains desired activity of the parent, e.g., the nuclease activity (except where the parent is a nickase or a dead Cpf1), and/or the ability to interact with a guide RNA and target DNA).

Other RNA-programmable DNA nickase that nicks the NTS, or a nuclease, including Cas-family enzymes (e.g., Cas9 or Cas12), TnpB-family, or IscB-family enzymes (Table A). See, e.g., Kapitonov et al., J Bacteriol. 2016 Mar. 1; 198 (5): 797-807; Karvelis et al., Nature. 2021; 599 (7886): 692-696 (TnpB); Koonin and Makarova, PLOS Biol. 2022 January; 20 (1): e3001481; Mingarro et al., Gene, 852:147064 (2023); Altae-Tran et al., Science. 2021 October; 374 (6563): 57-65 (TnpB and IscB); Meers et al., bioRxiv 2023.03.14.532601 (TnpB and IscB); Schuler et al., Science. 2022 Jun. 24; 376 (6600): 1476-1481; Kato et al., Nat Commun. 2022 Nov. 7; 13 (1): 6719. Nickases and catalytically inactive forms can be generated from wild type RNA-programmable DNA nucleases by the introduction of a mutation of a catalytic RuvC-II residue or a mutation of a catalytic HNH residue (Table A). For example, A. warmingii IscB nickases can include an H212A or E157A mutation; IscB nickases from other species can include corresponding mutations; see, e.g., WO 2022/087494. The nickase can also include one or more mutations that increase activity, reduce off-target effects, and/or alter protospacer adjacent motif (PAM) or target adjacent motif (TAM) specificity (Tables B and C). Exemplary Cas9 and Cas12 nickases and mutations are shown in Tables A-C.

TABLE A
List of Exemplary Cas9, Cas12a, and IscB Orthologs
(see WO2018218166 for references)

| | UniProt or GenBank | |
|---|---|---|
| | Accession Number | Nickase Mutations/Catalytic |
| Ortholog | or aa sequence | residues* |
| S. pyogenes Cas9 (SpCas9) | Q99ZW2.1 | D10A (RuvC), E762A (RuvC), |
| | | D839A (HNH), H840A (HNH), |
| | | N854A (HNH partial), N863A |
| | | (HNH), D983A (RuvC), D986A |
| | | (RuvC) |
| S. aureus Cas9 (SaCas9) | J7RUA5.1 | D10A (RuvC), D556A (HNH), |
| | | H557A (HNH), N571A (HNH), |
| | | N580 (HNH) |
| S. thermophilus Cas9 | G3ECR1.2 | D31A (RuvC), D867A (HNH), |
| (St1Cas9) | | H868A (HNH), N882A, (HNH), |
| | | N891A (HNH) |
| N. meningitidis Cas9 | CBA04028.1 | D16A (RuvC), D587A (HNH), |
| (Nme1sCas9) | | H588A (HNH), N602A (HNH), |
| | | N611A (HNH) |
| N. meningitidis DE10444 | WP_002230835.1 | D16A (RuvC), D587A (HNH), |
| Cas9 (Nme2Cas9) | | H588A (HNH), N602A (HNH), |
| | | N611A (HNH) |
| S. pasteurianus Cas9 | BAK30384.1 | D10A (RuvC), D598A (HNH), |
| (SpaCas9) | | H599A (HNH), N613A (HNH), |
| | | N622A (HNH) |
| C. jejuni Cas9 (CjCas9) | Q0P897.1 | D8A (RuvC), D558A (HNH), |
| | | H559A (HNH), N573A (HNH), |
| | | N582A (HNH) |
| F. novicida Cas9 (FnCas9) | A0Q5Y3.1 | D11A (RuvC), D968A (HNH), |
| | | H969A (HNH), N986A (HNH), |
| | | N995A (HNH) |
| P. lavamentivorans Cas9 | A7HP89.1 | D8A (RuvC), D600A (HNH), |
| (PlCas9) | | H601A (HNH), N615A (HNH), |
| | | N624A (HNH) |
| C. lari Cas9 (ClCas9) | G1UFN3.1 | D7A (RuvC), D566A (HNH), |
| | | H567 (HNH) |
| Pasteurella multocida Cas9 | Q9CLT2.1 | D13A (RuvC), D579A (HNH), |
| | | H580A (HNH), |
| F. novicida Cpf1 (FnCpf1) | A0Q7Q2.1 | D917A, E1006A, D1255A (all |
| | | RuvC) |
| M. bovoculi Cpf1 (MbCpf1) | WP_052585281.1 | D986A (RuvC) |
| A. sp. BV3L6 Cpf1 (AsCpf1) | U2UMQ6.1 | D90A8, E993A, Q1226A, |
| | | D1263A (all RuvC) |
| L. bacterium N2006 (LbCpf1) | A0A182DWE3.1 | D832A (RuvC) |
| ogeuIscB | Sequence below | E193A (RuvC) and H247A |
| | | (HNH) |

*for Cas9 and IscB enzymes, the RuvC domain nicks the non-target strand (NTS) DNA and the HNH domain nicks the target strand (TS) DNA. For Cas12a/Cpf1 or TnpB enzymes, the RuvC domain nicks both DNA strands. Mutations abrogate activity.

The sequence of ogeuIscB is as follows (from metagenome genome assembly, contig: NODE_25_length_150080_cov_8.882980; contig accession: OGEU01000025.1):

(SEQ ID NO: 12)
MAVVYVISKSGKPLMPTTRCGHVRILLKEGKARVVERKPFTIQLTYESA
EETQPLVLGIDPGRTNIGMSVVTESGESVFNAQIETRNKDVPKLMKDRK
QYRMAHRRLKRRCKRRRRAKAAGTAFEEGEKQRLLPGCFKPITCKSIRN
KEARFNNRKRPVGWLTPTANHLLVTHLNVVKKVQKILPVAKVVLELNRF
SFMAMNNPKVQRWQYQRGPLYGKGSVEEAVSMQQDGHCLFCKHGIDHYH
HVVPRRKNGSETLENRVGLCEEHHRLVHTDKEWEANLASKKSGMNKKYH
ALSVLNQIIPYLADQLADMFPGNFCVTSGQDTYLFREEHGIPKDHYLDA
YCIACSALTDAKKVSSPKGRPYMVHQFRRHDRQACHKANLNRSYYMGGK
LVATNRHKAMDQKTDSLEEYRAAHSAADVSKLTVKHPSAQYKDMSRIMP
GSILVSGEGKLFTLSRSEGRNKGQVNYFVSTEGIKYWARKCQYLRNNGG
LQIYV

TABLE B
List of Exemplary High Fidelity and/or PAM-relaxed RGN Orthologs

| Published HF/PAM- | | |
|---|---|---|
| RGN variants | PMID | Mutations* |
| S. pyogenes Cas9 | 26628643 | K810A/K1003A/R1060A eSpCas9(1.0); |
| (SpCas9) eSpCas9 | | K848A/K1003A/R1060A eSpCas9(1.1) |
| S. pyogenes Cas9 | 26735016 | N497A/R661A/Q695A/Q926A (SpCas9-HF1); L169A, |
| (SpCas9) HF1 | | Y450A, |
| S. pyogenes Cas9 | 28931002 | N692A, M694A, Q695A, H698A |
| (SpCas9) HypaCas9 | | |
| S. pyogenes Cas9 | 29431739 | M495V/Y515N/K526E/R661Q; |
| (SpCas9) evoCas9 | | (M495V/Y515N/K526E/R661S; |
| | | M495V/Y515N/K526E/R661L) |
| S. pyogenes Cas9 | 30082871 | R691A |
| (SpCas9) HiFi Cas9 | | |
| S. pyogenes Cas9 | 30082838 | F539S, M763I, K890N |
| (SpCas9) Sniper- | | |
| Cas9 | | |
| S. pyogenes Cas9 | 32144253 | Replacement of L1004-K1014 with two glycines |
| (SpCas9) Blackjack | | |
| S. pyogenes Cas9 | 32187529 | N690C, T769I, G915M, N980K |
| (SpCas9) LZ3 | | |
| S. pyogenes Cas9 | 35236982 | Y1010D, Y1013D, Y1016D, V1018D, R1019D, |
| (SpCas9) SuperFi- | | Q1027D, K1031D |
| Cas9 | | |
| S. pyogenes Cas9 | 36894722 | E1007L |
| (SpCas9) Sniper2L | | |
| S. pyogenes Cas9 | 36894722 | E1007P |
| (SpCas9) Sniper2P | | |
| S. pyogenes Cas9 | 36905119 | K526D |
| (SpCas9) rCas9HF | | |
| S. pyogenes Cas9 | 29512652 | A262T, R324L, S409I, E480K, E543D, M694I, |
| (SpCas9) xCas9 | | E1219V |
| S. pyogenes Cas9 | 30166441 | R1335V, L1111R, D1135V, G1218R, |
| (SpCas9) SpCas9-NG | | E1219F, A1322R, T1337R |
| S. pyogenes Cas9 | 26098369 | D1135V, R1335Q, T1337R (SpCas9-VQR); |
| (SpCas9) | | D1135V/G1218R/R1335E/T1337R (SpCas9-VRER); |
| VQR/VRER | | D1135E |
| S. pyogenes Cas9 | 26098369, | D1135V, G1218R, R1335Q, T1337R |
| (SpCas9) SpCas9- | 26735016 | |
| VRQR | | |
| S. aureus Cas9 | 26524662 | E782K/N968K/R1015H |
| (SaCas9)-KKH | | |
| enAsCas12a | U.S. Ser. No. 15/960,271 | One or more of: E174R, S170R, S542R, K548R, |
| | | K548V, N551R, N552R, K607R, K607H, e.g., |
| | | E174R/S542R/K548R, E174R/S542R/K607R, |
| | | E174R/S542R/K548V/N552R, S170R/S542R/K548R, |
| | | S170R/E174R, E174R/S542R, S170R/S542R, |
| | | E174R/S542R/K548R/N551R, E174R/S542R/K607H, |
| | | S170R/S542R/K607R, or |
| | | S170R/S542R/K548V/N552R |
| enAsCas12a-HF | U.S. Ser. No. 15/960,271 | One or more of: E174R, S542R, K548R, e.g., |
| | | E174R/S542R/K548R, E174R/S542R/K607R, |
| | | E174R/S542R/K548V/N552R, S170R/S542R/K548R, |
| | | S170R/E174R, E174R/S542R, S170R/S542R, |
| | | E174R/S542R/K548R/N551R, E174R/S542R/K607H, |
| | | S170R/S542R/K607R, or |
| | | S170R/S542R/K548V/N552R, with the addition of one |
| | | or more of: N282A, T315A, N515A and K949A |
| enLbCas12a(HF) | U.S. Ser. No. 15/960,271 | One or more of T152R, T152K, D156R, D156K, |
| | | Q529K, G532R, G532K, G532Q, K538R, K538V, |
| | | D541R, Y542R, M592A, K595R, K595H, K595S or |
| | | K595Q, e.g., D156R/G532R/K538R, |
| | | D156R/G532R/K595R, D156R/G532R/K538V/Y542R, |
| | | T152R/G532R/K538R, T152R/D156R, D156R/G532R, |
| | | T152R/G532R, D156R/G532R/K538R/D541R, |
| | | D156R/G532R/K595H, T152R/G532R/K595R, |
| | | T152R/G532R/K538V/Y542R, optionally with the |
| | | addition of one or more of: N260A, N256A, K514A, |
| | | D505A, K881A, S286A, K272A, K897A |
| enFnCas12a(HF) | U.S. Ser. No. 15/960,271 | One or more of T177A, K180R, K180K, E184R, |
| | | E184K, T604K, N607R, N607K, N607Q, K613R, |
| | | K613V, D616R, N617R, M668A, K671R, K671H, |
| | | K671S, or K671Q, e.g., E184R/N607R/K613R, |
| | | E184R/N607R/K671R, E184R/N607R/K613V/N617R, |
| | | K180R/N607R/K613R, K180R/E184R, E184R/N607R, |
| | | K180R/N607R, E184R/N607R/K613R/D616R, |
| | | E184R/N607R/K671H, K180R/N607R/K671R, |
| | | K180R/N607R/K613V/N617R, optionally with the |
| | | addition of one or more of: N305A, N301A, K589A, |
| | | N580A, K962A, S334A, K320A, K978A |
| chimeric Cas9 | 30718489 | S. aureus Cas9 with PAM interaction domain from |
| cCas9 | | SaCas9 orthologues, expands recognition and |
| | | targetability of NNVRRN, NNVACT, NNVATG, |
| | | NNVATT, NNVGCT, NNVGTG, and NNVGTT PAM |
| | | sequences |
| Streptococcus | doi.org/10.1 | Recognizes 5′-NAA-3′ PAM |
| macacae (Smac) | 101/429654 | |
| Cas9 NCTC 11558 | | |
| Spy-mac Cas9, | 32424114 | Recognizes 5′-NAA-3′ PAM |
| Smac-py Cas9, | | |
| iSpyMac | | |
| N. meningitidis | 30581144 | Recognizes N₄CC PAM |
| Nme2Cas9 | | |
| SpG Cas9 (SEQ-ID | 32217751; | SpCas9 variant capable of targeting NGN PAMs |
| 158) | WO | D1135L/S1136W/G1218K/E1219Q/R1335Q/T1337R |
| | 2021151085 | Also as SpG-HF1 in combination with |
| | | N497A/R661A/Q695A/Q926A |
| SpRY Cas9 (SEQ-ID | 32217751; | SpCas9 variant capable of targeting NRN > NYN PAMs |
| 157) | WO | SpRY(A61R/L1111R/D1135L/S1136W/G1218K/E1219Q/ |
| | 2021151085 | N1317R/A1322R/R1333P/R1335Q/T1337R); also |
| | | as SpRY-HF1 in combination with |
| | | N497A/R661A/Q695A/Q926A |
| S. pyogenes Cas9 | 32042170 | D10T(optional)/I322V/S409I/E427G/R654L/R753G/ |
| (SpCas9) SpCas9- | | R1114G/D1135N/E1219V/D1332N/R1335Q/T1337N/ |
| NRCH | | S1338T/H1349R |
| S. pyogenes Cas9 | 32042170 | D10T(optional)/I322V/S409I/E427G/R654L/R753G/ |
| (SpCas9) SpCas9- | | R1114G/D1135N/V1139A/D1180G/E1219V/Q1221H/ |
| NRRH | | A1320V/R1333K |
| S. pyogenes Cas9 | 32042170 | D10T(optional)/I322V/S409I/E427G/R654L/R753G/ |
| (SpCas9) SpCas9- | | R1114G/D1135N/D1180G/G1218S/E1219V/Q1221H/ |
| NRTH | | P1249S/E1253K/P1321S/D1332G/R1335L |

*predicted based on UniRule annotation on the UniProt database.

TABLE C
List of Exemplary SpCas9 Activity-Altering Mutations

| | PMID or | |
|---|---|---|
| Name of enzyme | patent ID | Mutations |
| S. pyogenes Cas9 (SpCas9) | 26098369, | L1111H, G1218R, T1337R |
| SpCas9-VRQR | 26735016 | |
| S. pyogenes Cas9 (SpCas9) | PCT/US2021/ | S55R, A61R, R221K, G366R, N394K, |
| (various mutations) | 014933 | G1104K, G1104R, L1111R, T1138K, |
| | | T1138R, V1139T, V1139S, K1151R, |
| | | K1153R, K1200R, G1218R, E1243K, |
| | | L1245V, E1253K, A1285R, A1285K, |
| | | K1289R, T1314R, N1317R, G1219R, |
| | | G1319H, A1320S, A1320T, A1322S, |
| | | A1322T, A1322R, D1332R, D1332K, |
| | | D1332H, D1332Q, D1332N, |
| | | A61R/N1317R, G1104K/N1317R, |
| | | A61R/G1104K, S55R/G1104K, |
| | | S55R/A61R, S55R/N1317R, |
| | | G1104K/T1314R, A61R/T1314R, |
| | | T1314R/N1317R, S55R/T1314R, |
| | | A61R/G1104R, G1104R/N1317R, |
| | | S55R/G1104R, G1104R/T1314R, |
| | | G366R/G1104K, S55R/G366R, |
| | | A61R/G366R, G366R/N1317R, |
| | | G366R/T1314R, G366R/G1104R, |
| | | G1104K/A1285R, A61R/A1285R, |
| | | A1285R/N1317R, S55R/A1285R, |
| | | A1285R/T1314R, G1104R/A1285R, |
| | | G366R/A1285R, G1104K/A1285K, |
| | | A61R/A1285K, A1285K/N1317R, |
| | | S55R/A1285K, A1285K/T1314R, |
| | | G1104R/A1285K, G366R/A1285K, |
| | | G1104K/D1332R, A61R/D1332R, |
| | | D1332R/N1317R, S55R/D1332R, |
| | | D1332R/T1314R, G1104R/D1332R, |
| | | G366R/D1332R, A1285K/D1332R, |
| | | A1285R/D1332R, |
| | | A61R/G1104K/N1317R, A61R/L1111R, |
| | | A61R/A1322R, A61R/L1111R/A1322R, |
| | | G1104K/L1111R, G1104K/A1322R, |
| | | G1104K/L1111R/A1322R, |
| | | N1317R/L1111R, N1317R/A1322R, |
| | | N1317R/L1111R/A1322R, |
| | | A61R/N1317R/L1111R, |
| | | A61R/N1317R/A1322R, |
| | | A61R/N1317R/L1111R/A1322R, |
| | | G1104K/N1317R/L1111R, |
| | | G1104K/N1317R/A1322R, |
| | | G1104K/N1317R/L 1111R/A1322R, |
| | | A61R/G1104K/L 1111R, |
| | | A61R/G1104K/A1322R, |
| | | A61R/G1104K/L1111R/A1322R, |
| | | S55R/L1111R, S55R/A1322R, |
| | | S55R/L1111R/A1322R, G366R/L1111R, |
| | | G366R/A1322R, G366R/L1111R/ |
| | | A1322R, N394K/L1111R, |
| | | N394K/A1322R, |
| | | N394K/L1111R/A1322R, |
| | | A1285K/L1111R, A1285K/A1322R, |
| | | A1285K/L1111R/A1322R, |
| | | D1332K/L1111R, D1332K/A1322R, |
| | | D1332K/L1111R/A1322R, R691A, |
| | | M495V, Y515N, K526E, R661Q, |
| | | R661L, R661S, Y450A/Q695A, |
| | | L169A/Q695A, Q695A/Q926A, |
| | | Q695A/D1135E, Q926A/D1135E, |
| | | Y450A/D1135E, L169A/Y450A/Q695A, |
| | | L169A/Q695A/Q926A, |
| | | Y450A/Q695A/Q926A, |
| | | R661A/Q695A/Q926A, |
| | | N497A/Q695A/Q926A, |
| | | Y450A/Q695A/D1135E, |
| | | Y450A/Q926A/D 1135E, |
| | | Q695A/Q926A/D1135E, |
| | | L169A/Y450A/Q695A/Q926A, |
| | | L169A/R661A/Q695A/Q926A, |
| | | Y450A/R661A/Q695A/Q926A, |
| | | N497A/Q695 A/Q926A/D1135E, |
| | | R661A/Q695A/Q926A/D1135E, and |
| | | Y450A/Q695A/Q926A/D1135E; |
| | | N692A/M694A/Q695A/H698A, |
| | | N692A/M694A/Q695A/H698A/Q926A; |
| | | N692A/M694A/Q695A/Q926A; |
| | | N692A/M694A/H698A/Q926A; |
| | | N692A/Q695A/H698A/Q926A; |
| | | M694A/Q695A/H698A/Q926A; |
| | | N692A/Q695A/H698A; |
| | | N692A/M694A/Q695A; |
| | | N692A/H698A/Q926A; |
| | | N692A/M694A/Q926A; |
| | | N692A/M694A/H698A; |
| | | M694A/Q695A/H698A; |
| | | M694A/Q695A/Q926A; |
| | | Q695A/H698A/Q926A; |
| | | G582A/V583A/E584A/D585A/N588A/ |
| | | Q926A; |
| | | G582A/V583A/E584A/D585A/N588A; |
| | | T657A/G658A/W659A/R661A/Q926A; |
| | | T657A/G658A/W659A/R661A; |
| | | F491A/M495A/T496A/N497A/Q926A; |
| | | F491A/M495A/T496A/N497A; |
| | | K918A/V922A/R925A/Q926A; or |
| | | 918A/V922A/R925A; K855A; |
| | | K810A/K1003A/R1060A; |
| | | K848A/K1003A/R1060A; |
| | | M495V/Y515N/K526E/R661Q; |
| | | M495V/Y515N/K526E/R661L; or |
| | | M495V/Y515N/K526E/R661S |
| S. pyogenes Cas9 (SpCas9) | 32217751 | A61R, G1104K (not present in final |
| SpRY | | SpRY enzyme), L1111R, N1317R, |
| | | A1322R |
| S. pyogenes Cas9 (SpCas9) | 30166441 | L1111R, A1322R |
| SpCas9-NG | | |
| S. pyogenes Cas9 (SpCas9) | 29203891 | R221K, L244Q, N349K, E1243K, |
| (various mutations) | | L1245V, E1253K |
| S. pyogenes Cas9 (SpCas9) | 26990992 | N1286Q, I1331F, D1332K, |
| SpCas9-QQR | | |
| S. pyogenes Cas9 (SpCas9) | 25207793 | D147Y, P411T |
| iCas9 | | |
| S. pyogenes Cas9 (SpCas9) | 36917274 | L1206P |
| SpCas9-L1206P | | |

### Guide RNAs

The methods can include the delivery of a Cas variant protein (or nucleic acid encoding the Cas Variant and one or more guide RNA (gRNAs) bearing various spacer sequences that target the dCas9 to the repeat expansion region. Preferably the gRNA binds to an exon/repeat border. A number of exemplary Cas enzyme target sites and corresponding gRNA spacer sequences are provided in Tables 1 and 2, with the corresponding Cas variant indicated.

For the gRNA spacer sequences in Table 1, exemplary spacer sequences are shown; the 5′ end of the spacer may be extended or substituted to include alternate nucleotide compositions to modify transcription from polIII promoters. The spacer sequence can be, e.g., 20 nucleotides (nt) with a matched 5′ guanine (G), 20 nt with a mismatched 5′G, 20 nt with matched or mismatched alternate nts, 21 nt with an extended matched or mismatched 5′G or other nts, or 22 nt with extended matched or mismatched nts optionally including a 5′G.

In Table 2, the Cpf1/Cas12a target sites have exemplary 23 nt spacer sequences; the spacer sequences for the Cpf1/Cas12a crRNAs may be truncated to 22, 21, 20, 19, 18, or 17 nt (by removing bases from the 3′ PAM distal end of the spacer).

TABLE 1
HTT exon 1 target sites and gRNA spacers for Cas9 enzymes

| | gRNA | target | | | | Loca-tion of | |
|---|---|---|---|---|---|---|---|
| site | plasmid | site with | | gRNA spacer | Spacer | target site | Cas |
| ID | ID | PAM | PAM | sequence | length | in HTT | Variant |
| SpCas9- | RTW3 | CTGCTGCTG | GG | GCTGCTGCTGG | 21 (+1 | 5′ edge of | WT SpCas9 |
| NGG-1 | | GAAGGACTT | G | AAGGACTTGA | 5′G) | CAG repeat | |
| | | GAGGG | | (SEQ ID NO: | | BOTTOM | |
| | | (SEQ ID | | 14) | | | |
| | | NO: 13) | | | | | |
| SpCas9- | RTW22 | GCTGCTGCT | AG | GCTGCTGCTGG | 20 | 5′ edge of | WT SpCas9 |
| NGG-2 | | GGAAGGACT | G | AAGGACTTG | | CAG repeat | |
| | | TGAGG | | (SEQ ID NO: | | BOTTOM | |
| | | (SEQ ID | | 16) | | | |
| | | NO: 15) | | | | | |
| SpCas9- | RTW5 | CTGCTGCTG | AG | GCTGCTGCTGC | 21 (+1 | 5′ edge of | WT SpCas9 |
| NGG-3 | | CTGCTGCTG | G | TGCTGCTGGA | 5′G) | CAG repeat | |
| | | GAAGG | | (SEQ ID NO: | | BOTTOM | |
| | | (SEQ ID | | 10) | | | |
| | | NO: 17) | | | | | |
| SpCas9- | RTW24 | GCTGCTGCT | TG | GCTGCTGCTGC | 20 | 5′ edge of | WT SpCas9 |
| NGG-4 | | GCTGCTGCT | G | TGCTGCTGC | | CAG repeat | |
| | | GCTGG | | (SEQ ID NO: | | BOTTOM | |
| | | (SEQ ID | | 174) | | | |
| | | NO: 18) | | | | | |
| SpCas9- | HES223 | GAAGGACTT | AG | GAAGGACTTGA | 20 | fully outside | WT SpCas9 |
| NGG-5 | | GAGGGACTC | G | GGGACTCGA | | 5′ repeat | |
| | | GAAGG | | (SEQ ID NO: | | BOTTOM | |
| | | (SEQ ID | | 20) | | | |
| | | NO: 19) | | | | | |
| SpCas9- | HES224 | GGCGGCGGC | CG | GGCGGCGGCGG | 20 | 5′ end of | WT SpCas9 |
| NGG-6 | | GGCGGCGGT | G | CGGCGGTGG | | Proline | |
| | | GGCGG | | (SEQ ID NO: | | repeat BOTTOM | |
| | | (SEQ ID | | 22) | | | |
| | | NO: 21) | | | | | |
| SpCas9- | HES225 | GGCGGCGGC | TG | GGCGGCGGCGG | 20 | fully in | WT SpCas9 |
| NGG-7 | | GGCGGCGGC | G | CGGCGGCGG | | Proline | |
| | | GGTGG | | (SEQ ID NO: | | repeat BOTTOM | |
| | | (SEQ ID | | 24) | | | |
| | | NO: 23) | | | | | |
| SpCas9- | HES226 | GGAAGCTGA | CG | GGAAGCTGAGG | 20 | 3′ end of | WT SpCas9 |
| NGG-8 | | GGAGGCGGC | G | AGGCGGCGG | | Proline | |
| | | GGCGG | | (SEQ ID NO: | | repeat BOTTOM | |
| | | (SEQ ID | | 26) | | | |
| | | NO: 25) | | | | | |
| SpCas9- | HES227 | TGAGGAAGC | CG | GTGAGGAAGCT | 21 (+1 | 3′ end of | WT SpCas9 |
| NGG-9 | | TGAGGAGGC | G | GAGGAGGCGG | 5 ′G) | Proline | |
| | | GGCGG | | (SEQ ID NO: | | repeat BOTTOM | |
| | | (SEQ ID | | 28) | | | |
| | | NO: 27) | | | | | |
| SpCas9- | HES228 | GGCTGAGGA | CG | GGCTGAGGAAG | 20 | 3′ end of | WT SpCas9 |
| NGG-10 | | AGCTGAGGA | G | CTGAGGAGG | | Proline | |
| | | GGCGG | | (SEQ ID NO: | | repeat BOTTOM | |
| | | (SEQ ID | | 30) | | | |
| | | NO: 29) | | | | | |
| SpCas9- | HES240 | GGCGGCTGA | AG | GGCGGCTGAGG | 20 | 3′ end of | WT SpCas9 |
| NGG-11 | | GGAAGCTGA | G | AAGCTGAGG | | Proline | |
| | | GGAGG | | (SEQ ID NO: | | repeat BOTTOM | |
| | | (SEQ ID | | 32) | | | |
| | | NO: 31) | | | | | |
| SpCas9- | HES241 | GGCGGCGGC | AG | GGCGGCGGCTG | 20 | fully out 3′ | WT SpCas9 |
| NGG-12 | | TGAGGAAGC | G | AGGAAGCTG | | Pro repeat | |
| | | TGAGG | | (SEQ ID NO: | | BOTTOM | |
| | | (SEQ ID | | 34) | | | |
| | | NO: 33) | | | | | |
| SpCas9- | HES242 | TGAAGGCCT | AA | GTGAAGGCCTT | 21 (+1 | fully outside | WT or |
| NAG-1 | | TCGAGTCCC | G | CGAGTCCCTC | 5′G) | 5′ repeat TOP | SpRY |
| | | TCAAG | | (SEQ ID NO: | | | |
| | | (SEQ ID | | 36) | | | |
| | | NO : 35) | | | | | |
| SpCas9- | HES243 | TCGAGTCCC | CA | GTCGAGTCCCT | 21 (+1 | 5′ edge of | WT or |
| NAG-2 | | TCAAGTCCT | G | CAAGTCCTTC | 5′G) | CAG repeat | SpRY |
| | | TCCAG | | (SEQ ID NO: | | TOP | |
| | | (SEQ ID | | 38) | | | |
| | | NO: 37) | | | | | |
| SpCas9- | HES244 | AGTCCCTCA | CA | GAGTCCCTCAA | 21 (+1 | 5′ edge of | WT or SpRY |
| NAG-3 | | AGTCCTTCC | G | GTCCTTCCAG | 5′G) | CAG repeat | |
| | | AGCAG | | (SEQ ID NO: | | TOP | |
| | | (SEQ ID | | 40) | | | |
| | | NO: 39) | | | | | |
| SpCas9- | HES245 | CCCTCAAGT | CA | GCCCTCAAGTC | 21 (+1 | 5′ edge of | WT or SpRY |
| NAG-4 | | CCTTCCAGC | G | CTTCCAGCAG | 5′G) | CAG repeat | |
| | | AGCAG | | (SEQ ID NO: | | TOP | |
| | | (SEQ ID | | 42) | | | |
| | | NO: 41) | | | | | |
| SpCas9- | HES246 | TCAAGTCCT | CA | GTCAAGTCCTT | 21 (+1 | 5′ edge of | WT or |
| NAG-5 | | TCCAGCAGC | G | CCAGCAGCAG | 5′G) | CAG repeat | SpRY |
| | | AGCAG | | (SEQ ID NO: | | TOP | |
| | | (SEQ ID | | 44) | | | |
| | | NO: 43) | | | | | |
| SpCas9- | HES251 | AGTCCTTCC | CA | GAGTCCTTCCA | 21 (+1 | 5′ edge of | WT or |
| NAG-6 | | AGCAGCAGC | G | GCAGCAGCAG | 5′G) | CAG repeat | SpRY |
| | | AGCAG | | (SEQ ID NO: | | TOP | |
| | | (SEQ ID | | 46) | | | |
| | | NO: 45) | | | | | |
| SpCas9- | HES292 | CCTTCCAGC | CA | GCCTTCCAGCA | 21 (+1 | 5′ edge of | WT or SpRY |
| NAG-7 | | AGCAGCAGC | G | GCAGCAGCAG | 5′G) | CAG repeat | |
| | | AGCAG | | (SEQ ID NO: | | TOP | |
| | | SEQ ID | | 48) | | | |
| | | NO: 47) | | | | | |
| SpCas9- | HES252 | TCCAGCAGC | CA | GTCCAGCAGCA | 21 (+1 | 5′ edge of | WT or |
| NAG-8 | | AGCAGCAGC | G | GCAGCAGCAG | 5′G) | CAG repeat | SpRY |
| | | AGCAG | | (SEQ ID NO: | | TOP | |
| | | (SEQ ID | | 50) | | | |
| | | NO: 49) | | | | | |
| SpCas9- | HES253 | AGCAGCAGC | CA | GAGCAGCAGCA | 21 (+1 | fully inside | WT or |
| NAG-9 | | AGCAGCAGC | G | GCAGCAGCAG | 5′G) | CAG repeat | SpRY |
| | | AGCAG | | (SEQ ID NO: | | TOP | |
| | | SEQ | | 5) | | | |
| | | NO: 51) | | | | | |
| SpCas9- | HES254 | CGCCGCCGC | CA | GCGCCGCCGCC | 21 (+1 | 3′ end of | WT or |
| NAG-10 | | CGCCGCCTC | G | GCCGCCTCCT | 5′G) | Proline | SpRY |
| | | CTCAG | | (SEQ ID NO: | | repeat TOP | |
| | | (SEQ ID | | 7) | | | |
| | | NO: 52) | | | | | |
| SpCas9- | HES255 | CGCCGCCTC | CA | GCGCCGCCTCC | 21 (+1 | 3′ end of | WT or SpRY |
| NAG-11 | | CTCAGCTTC | G | TCAGCTTCCT | 5′G) | Proline | |
| | | CTCAG | | (SEQ ID NO: | | repeat TOP | |
| | | (SEQ ID | | 54) | | | |
| | | NO: 53) | | | | | |
| SpCas9- | HES256 | TGCTGCTGC | GA | GTGCTGCTGCT | 21 (+1 | 5′ edge of | WT or |
| NAG-12 | | TGGAAGGAC | G | GGAAGGACTT | 5′G) | CAG repeat | SpRY |
| | | TTGAG | | (SEQ ID NO: | | BOTTOM | |
| | | (SEQ ID | | 6) | | | |
| | | NO: 55) | | | | | |
| SpCas9- | HES540 | GCTGCTGCT | AA | GCTGCTGCTGC | 20 | 5′ edge of | WT or |
| NAG-13 | | GCTGCTGCT | G | TGCTGCTGG | | CAG repeat | SpRY |
| | | GGAAG | | (SEQ ID NO: | | BOTTOM | |
| | | (SEQ ID | | 57) | | | |
| | | NO: 56) | | | | | |
| SpCas9- | HES580 | GGAAGGACT | AA | GGAAGGACTTG | 20 | 5′ edge of | WT or |
| NAG-14 | | TGAGGGACT | G | AGGGACTCG | | CAG repeat | SpRY |
| | | CGAAG | | (SEQ ID NO: | | BOTTOM | |
| | | (SEQ ID | | 59) | | | |
| | | NO: 58) | | | | | |
| SpCas9- | HES257 | CGAGTCCCT | AG | GCGAGTCCCTC | 21 (+1 | fully outside | SpCas9- |
| NGC-1 | | CAAGTCCTT | C | AAGTCCTTCC | 5′G) | 5′ repeat TOP | NGC |
| | | CCAGC | | (SEQ ID NO: | | | |
| | | (SEQ ID | | 61) | | | |
| | | NO: 60) | | | | | |
| SpCas9- | HES258 | GTCCCTCAA | AG | GTCCCTCAAGT | 20 | 5′ edge of | SpCas9- |
| NGC-2 | | GTCCTTCCA | C | CCTTCCAGC | | CAG repeat | NGC |
| | | GCAGC | | (SEQ ID NO: | | TOP | |
| | | (SEQ ID | | 63) | | | |
| | | NO: 62) | | | | | |
| SpCas9- | HES259 | CCTCAAGTC | AG | GCCTCAAGTCC | 21 (+1 | 5′ edge of | SpCas9- |
| NGC-3 | | CTTCCAGCA | C | TTCCAGCAGC | 5′G) | CAG repeat | NGC |
| | | GCAGC | | (SEQ ID NO: | | TOP | |
| | | (SEQ ID | | 65) | | | |
| | | NO: 64) | | | | | |
| SpCas9- | HES260 | CAAGTCCTT | AG | GCAAGTCCTTC | 21 (+1 | 5′ edge of | SpCas9- |
| NGC-4 | | CCAGCAGCA | C | CAGCAGCAGC | 5′G) | CAG repeat | NGC |
| | | GCAGC | | (SEQ ID NO: | | TOP | |
| | | (SEQ ID | | 67) | | | |
| | | NO: 66) | | | | | |
| SpCas9- | HES261 | GTCCTTCCA | AG | GTCCTTCCAGC | 20 | 5′ edge of | SpCas9- |
| NGC-5 | | GCAGCAGCA | C | AGCAGCAGC | | CAG repeat | NGC |
| | | GCAGC | | (SEQ ID NO: | | TOP | |
| | | (SEQ ID | | 69) | | | |
| | | NO: 68) | | | | | |
| SpCas9- | HES262 | CTTCCAGCA | AG | GCTTCCAGCAG | 21 (+1 | 5′ edge of | SpCas 9- |
| NGC-6 | | GCAGCAGCA | C | CAGCAGCAGC | 5′G) | CAG repeat | NGC |
| | | GCAGC | | (SEQ ID NO: | | TOP | |
| | | (SEQ ID | | 71) | | | |
| | | NO: 70) | | | | | |
| SpCas9- | HES2 63 | CCAGCAGCA | AG | GCCAGCAGCAG | 21 (+1 | 5′ edge of | SpCas9- |
| NGC-7 | | GCAGCAGCA | C | CAGCAGCAGC | 5′G) | CAG repeat | NGC |
| | | GCAGC | | (SEQ ID NO: | | TOP | |
| | | (SEQ ID | | 73) | | | |
| | | NO: 72) | | | | | |
| SpCas9- | HES264 | GCAGCAGCA | AG | GCAGCAGCAGC | 20 | fully inside | SpCas9- |
| NGC-8 | | GCAGCAGCA | C | AGCAGCAGC | | CAG repeat | NGC |
| | | GCAGC | | (SEQ ID NO: | | TOP | |
| | | (SEQ ID | | 3) | | | |
| | | NO: 74) | | | | | |
| SpCas9- | HES2 65 | GCTGCTGCT | TG | GCTGCTGCTGC | 20 | fully inside | SpCas9- |
| NGC-9 | | GCTGCTGCT | C | TGCTGCTGC | | CAG repeat | NGC |
| | | GCTGC | | (SEQ ID NO: | | BOT | |
| | | (SEQ ID | | 8) | | | |
| | | NO: 75) | | | | | |
| SpCas9- | HES266 | GTTGCTGCT | TG | GTTGCTGCTGC | 20 | 3 edge of CAG | SpCas9- |
| NGC-10 | | GCTGCTGCT | C | TGCTGCTGC | | repeat BOTTOM | NGC |
| | | GCTGC | | (SEQ ID NO: | | | |
| | | (SEQ ID | | 77) | | | |
| | | NO: 76) | | | | | |
| SpCas9- | HES267 | GCTGTTGCT | TG | GCTGTTGCTGC | 20 | 3 edge of CAG | SpCas9- |
| NGC-11 | | GCTGCTGCT | C | TGCTGCTGC | | repeat BOTTOM | NGC |
| | | GCTGC | | (SEQ ID NO: | | | |
| | | (SEQ ID | | 79) | | | |
| | | NO: 78) | | | | | |
| SpCas9- | HES268 | GCGGCTGTT | TG | GCGGCTGTTGC | 20 | 3 edge of CAG | SpCas9- |
| NGC-12 | | GCTGCTGCT | C | TGCTGCTGC | | repeat BOTTOM | NGC |
| | | GCTGC | | (SEQ ID NO: | | | |
| | | (SEQ ID | | 81) | | | |
| | | NO: 80) | | | | | |
| SpCas9- | HES269 | GTGGCGGCT | TG | GTGGCGGCTGT | 20 | 3 edge of CAG | SpCas9- |
| NGC-13 | | GTTGCTGCT | C | TGCTGCTGC | | repeat BOTTOM | NGC |
| | | GCTGC | | (SEQ ID NO: | | | |
| | | (SEQ ID | | 83) | | | |
| | | NO: 82) | | | | | |
| SpCas9- | HES270 | GCGGTGGCG | TG | GCGGTGGCGGC | 20 | 3 edge of CAG | SpCas9- |
| NGC-14 | | GCTGTTGCT | C | TGTTGCTGC | | repeat BOTTOM | NGC |
| | | GCTGC | | (SEQ ID NO: | | | |
| | | (SEQ ID | | 85) | | | |
| | | NO: 84) | | | | | |
| SpCas9- | HES271 | GCGGCGGTG | TG | GCGGCGGTGGC | 20 | 3 edge of CAG | SpCas 9- |
| NGC-15 | | GCGGCTGTT | C | GGCTGTTGC | | repeat BOTTOM | NGC |
| | | GCTGC | | (SEQ ID NO: | | | |
| | | (SEQ ID | | 87) | | | |
| | | NO: 86) | | | | | |
| SpCas9- | HES272 | GCGGCGGCG | TG | GCGGCGGCGGT | 20 | 3 edge of CAG | SpCas9- |
| NGC-16 | | GTGGCGGCT | C | GGCGGCTGT | | repeat BOTTOM | NGC |
| | | GTTGC | | (SEQ ID NO: | | | |
| | | (SEQ ID | | 89) | | | |
| | | NO: 88) | | | | | |
| SpCas9- | HES273 | CGGCGGCGG | GC | GCGGCGGCGGC | 21 (+1 | 5′ end of | SpCas9- |
| NGC-17 | | CGGCGGCGG | G | GGCGGCGGTG | 5′G) | Proline | NGC |
| | | TGGCG | | (SEQ ID NO: | | repeat BOTTOM | |
| | | (SEQ ID | | 91) | | | |
| | | NO: 90) | | | | | |
| SpCas9- | HES274 | GAGGCGGCG | GG | GAGGCGGCGGC | 20 | 3′ end of | SpCas9- |
| NGC-18 | | GCGGCGGCG | C | GGCGGCGGC | | Proline | NGC |
| | | GCGGC | | (SEQ ID NO: | | repeat BOTTOM | |
| | | (SEQ ID | | 93) | | | |
| | | NO: 92) | | | | | |
| SpCas9- | HES275 | GCAGCAGCA | AG | GCAGCAGCAGC | 20 | 3 edge of CAG | SpCas9- |
| NGC-19 | | GCAGCAGCA | C | AGCAGCAAC | | repeat TOP | NGC |
| | | ACAGC | | (SEQ ID NO: | | | |
| | | (SEQ ID | | 95) | | | |
| | | NO: 94) | | | | | |
| SpCas9- | HES276 | GCAGCAGCA | CG | GCAGCAGCAGC | 20 | 3 edge of CAG | SpCas9- |
| NGC-20 | | GCAGCAACA | C | AGCAACAGC | | repeat TOP | NGC |
| | | GCCGC | | (SEQ ID NO: | | | |
| | | (SEQ ID | | 97) | | | |
| | | NO: 96) | | | | | |
| SpCas9- | HES277 | GCAGCAGCA | CG | GCAGCAGCAAC | 20 | 3 edge of CAG | SpCas9- |
| NGC-21 | | ACAGCCGCC | C | AGCCGCCAC | | repeat TOP | NGC |
| | | ACCGC | | (SEQ ID NO: | | | |
| | | (SEQ ID | | 99) | | | |
| | | NO: 98) | | | | | |
| SpCas9- | HES278 | GCAGCAACA | CG | GCAGCAACAGC | 20 | 3 edge of CAG | SpCas9- |
| NGC-22 | | GCCGCCACC | C | CGCCACCGC | | repeat TOP | NGC |
| | | GCCGC | | (SEQ ID NO: | | | |
| | | (SEQ ID | | 101) | | | |
| | | NO: 100) | | | | | |
| SpCas9- | HES279 | GCAACAGCC | CG | GCAACAGCCGC | 20 | 3 edge of CAG | SpCas9- |
| NGC-23 | | GCCACCGCC | C | CACCGCCGC | | repeat TOP | NGC |
| | | GCCGC | | (SEQ ID NO: | | | |
| | | (SEQ ID | | 103) | | | |
| | | NO: 102) | | | | | |
| SpCas9- | HES280 | ACAGCCGCC | CG | GACAGCCGCCA | 21 (+1 | 3 edge of CAG | SpCas 9- |
| NGC-24 | | ACCGCCGCC | C | CCGCCGCCGC | 5′G) | repeat TOP | NGC |
| | | GCCGC | | (SEQ ID NO: | | | |
| | | (SEQ ID | | 105) | | | |
| | | NO: 104) | | | | | |
| SpCas9- | HES281 | GCGGCGGCG | TG | GCGGCGGCGGC | 20 | 3 edge of CAG | SpCas9- |
| NGT-1 | | GCGGTGGCG | T | GGTGGCGGC | | repeat BOTTOM | NGT |
| | | GCTGT | | (SEQ ID NO: | | | |
| | | (SEQ ID | | 107) | | | |
| | | NO: 106) | | | | | |
| SpCas9- | HES282 | CTGGAAGGA | CG | GCTGGAAGGAC | 21 (+1 | 5′ edge of | SpCas9- |
| NGA-1 | | CTTGAGGGA | A | TTGAGGGACT | 5′G) | CAG repeat | NGA |
| | | CTCGA | | (SEQ ID NO: | | BOTTOM | |
| | | (SEQ ID | | 109) | | | |
| | | NO: 108) | | | | | |
| SpCas9- | HES283 | TGCTGCTGG | GG | GTGCTGCTGGA | 21 (+1 | 5′ edge of | SpCas9- |
| NGA-2 | | AAGGACTTG | A | AGGACTTGAG | 5′G) | CAG repeat | NGA |
| | | AGGGA | | (SEQ ID NO: | | BOTTOM | |
| | | (SEQ ID | | 4) | | | |
| | | NO: 110) | | | | | |
| SpCas9- | HES284 | CTGCTGCTG | TG | GCTGCTGCTGC | 21 (+1 | 5′ edge of | SpCas9- |
| NGA-3 | | CTGGAAGGA | A | TGGAAGGACT | 5′G) | CAG repeat | NGA |
| | | CTTGA | | (SEQ ID NO: | | BOTTOM | |
| | | (SEQ ID | | 9) | | | |
| | | NO: 111) | | | | | |
| SpCas9- | HES285 | TGCTGCTGC | GG | GTGCTGCTGCT | 21 (+1 | 5′ edge of | SpCas 9- |
| NGA-4 | | TGCTGCTGG | A | GCTGCTGGAA | 5′G) | CAG repeat | NGA |
| | | AAGGA | | (SEQ ID NO: | | BOTTOM | |
| | | (SEQ ID | | 11) | | | |
| | | NO: 112) | | | | | |
| SpCas9- | HES286 | CTGCTGCTG | GG | GCTGCTGCTGC | 21 (+1 | 5′ edge of | SpCas 9- |
| NGA-5 | | CTGCTGCTG | A | TGCTGCTGCT | 5′G) | CAG repeat | NGA |
| | | CTGGA | | (SEQ ID NO: | | BOTTOM | |
| | | (SEQ ID | | 114 | | | |
| | | NO: 113) | | | | | |
| SpCas9- | HES538 | TGCTGCTGC | GA | GTGCTGCTGCT | 20 (+1 | 5′ edge of | SpRY |
| NAA-1 | | TGCTGCTGC | A | GCTGCTGCTG | 5 ′G) | CAG repeat | |
| | | TGGAA | | (SEQ ID NO: | | BOTTOM | |
| | | (SEQ ID | | 116) | | | |
| | | NO: 115) | | | | | |
| SpCas9- | HES546 | TGGAAGGAC | GA | GTGGAAGGACT | 20 (+1 | 5′ edge of | SpRY |
| NAA-2 | | TTGAGGGAC | A | TGAGGGACTC | 5′G) | CAG repeat | |
| | | TCGAA | | (SEQ ID NO: | | BOTTOM | |
| | | (SEQ ID | | 118) | | | |
| | | NO: 117) | | | | | |
| SpCas9- | HES542 | GCTGCTGGA | GA | GCTGCTGGAAG | 20 | 5′ edge of | SpRY |
| NAC-1 | | AGGACTTGA | C | GACTTGAGG | | CAG repeat | |
| | | GGGAC | | (SEQ ID NO: | | BOTTOM | |
| | | (SEQ ID | | 120) | | | |
| | | NO: 119) | | | | | |
| SpCas9- | HES545 | GCTGCTGCT | GA | GCTGCTGCTGC | 20 | 5′ edge of | SpRY |
| NAC-2 | | GCTGCTGGA | C | TGCTGGAAG | | CAG repeat | |
| | | AGGAC | | (SEQ ID NO: | | BOTTOM | |
| | | (SEQ ID | | 122) | | | |
| | | NO: 121) | | | | | |
| SpCas9- | LLH457 | GCTGGAAGG | TC | GCTGGAAGGAC | 20 | 5′ edge of | SpRY |
| NYN-1 | | ACTTGAGGG | G | TTGAGGGAC | | CAG repeat | |
| | | ACTCG | | (SEQ ID NO: | | BOTTOM | |
| | | (SEQ ID | | 124) | | | |
| | | NO: 123) | | | | | |
| SpCas9- | LLH459 | TGCTGGAAG | CT | GTGCTGGAAGG | 21 (+1 | 5′ edge of | SpRY |
| NYN-2 | | GACTTGAGG | C | ACTTGAGGGA | 5′G) | CAG repeat | |
| | | GACTC | | (SEQ ID NO: | | BOTTOM | |
| | | (SEQ ID | | 126) | | | |
| | | NO: 125) | | | | | |
| SpCas9- | LLH461 | CTGCTGGAA | AC | GCTGCTGGAAG | 21 (+1 | 5′ edge of | SpRY |
| NYN-3 | | GGACTTGAG | T | GACTTGAGGG | 5′G) | CAG repeat | |
| | | GGACT | | (SEQ ID NO: | | BOTTOM | |
| | | ( SEQ ID | | 128) | | | |
| | | NO: 127) | | | | | |
| SpCas9- | LLH463 | GCTGCTGCT | TT | GCTGCTGCTGC | 20 | 5′ edge of | SpRY |
| NYN-4 | | GCTGGAAGG | G | TGGAAGGAC | | CAG repeat | |
| | | ACTTG | | (SEQ ID NO: | | BOTTOM | |
| | | (SEQ ID | | 130) | | | |
| | | NO: 129) | | | | | |
| SpCas9- | LLH465 | TGCTGCTGC | CT | GTGCTGCTGCT | 21 (+1 | 5′ | SpRY |
| NYN-5 | | TGCTGGAAG | T | GCTGGAAGGA | 5′G) | edge of | |
| | | GACTT | | (SEQ ID NO: | | CAG repeat | |
| | | (SEQ ID | | 132) | | BOTTOM | |
| | | NO: 131) | | | | | |
| SpCas9- | LLH468 | CTGCTGCTG | AC | GCTGCTGCTGC | 21 (+1 | 5′ edge of | SpRY |
| NYN-6 | | CTGCTGGAA | T | TGCTGGAAGG | 5′G) | CAG repeat | |
| | | GGACT | | (SEQ ID NO: | | BOTTOM | |
| | | (SEQ ID | | 134) | | | |
| | | NO: 133) | | | | | |

TABLE 2
HTT exon 1 target sites and crRNA spacers for Cas12a enzymes

| | | full | | | | |
|---|---|---|---|---|---|---|
| site | plasmid | target | | spacer | spacer | |
| ID | ID | with PAM | PAM | sequence only | length | Location |
| Cas12a- | RTW8 | CTTCGAGTC | CTTC | GAGTCCCTCAAGTC | 23 | 5′ edge of |
| 1 | | CCTCAAGTC | | CTTCCAGCA (SEQ | | CAG repeat |
| | | CTTCCAGCA | | ID NO: 136) | | TOP |
| | | (SEQ ID | | | | |
| | | NO: 135) | | | | |
| Cas12a- | RTW9 | TTCGAGTCC | TTCG | AGTCCCTCAAGTCC | 23 | 5′ edge of |
| 2 | | CTCAAGTCC | | TTCCAGCAG (SEQ | | CAG repeat |
| | | TTCCAGCAG | | ID NO: 175) | | TOP |
| | | (SEQ ID | | | | |
| | | NO: 137) | | | | |
| Cas12a- | HES294 | TCCCTCAAG | TCCC | TCAAGTCCTTCCAG | 23 | 5′ edge of |
| 3 | | TCCTTCCAG | | CAGCAGCAG (SEQ | | CAG repeat |
| | | CAGCAGCAG | | ID NO: 176) | | TOP |
| | | (SEQ ID | | | | |
| | | NO: 138) | | | | |
| Cas12a- | HES295 | TCCTTCCAG | TCCT | TCCAGCAGCAGCAG | 23 | 5′ edge of |
| 4 | | CAGCAGCAG | | CAGCAGCAG (SEQ | | CAG repeat |
| | | CAGCAGCAG | | ID NO: 177) | | TOP |
| | | (SEQ ID | | | | |
| | | NO: 139) | | | | |
| Cas12a- | RTW53 | CTTCCAGCA | CTTC | CAGCAGCAGCAGCA | 23 | 5′ edge of |
| 5 | | GCAGCAGCA | | GCAGCAGCA (SEQ | | CAG repeat |
| | | GCAGCAGCA | | ID NO: 141) | | TOP |
| | | (SEQ ID | | | | |
| | | NO: 140) | | | | |
| Cas12a- | RTW30 | TTCCAGCAG | TTCC | AGCAGCAGCAGCAG | 23 | 5′ edge of |
| 6 | | CAGCAGCAG | | CAGCAGCAG (SEQ | | CAG repeat |
| | | CAGCAGCAG | | ID NO: 178) | | TOP |
| | | (SEQ ID | | | | |
| | | NO: 142) | | | | |
| Cas12a- | HES296 | GTTGCTGCT | GTTG | CTGCTGCTGCTGCT | 23 | 3 edge of |
| 7 | | GCTGCTGCT | | GCTGCTGCT (SEQ | | CAG repeat |
| | | GCTGCTGCT | | ID NO: 144) | | BOTTOM |
| | | (SEQ ID | | | | |
| | | NO: 143) | | | | |
| Cas12a- | HES298 | CTTGAGGGA | CTTG | AGGGACTCGAAGGC | 23 | fully |
| 8 | | CTCGAAGGC | | CTTCATCAG (SEQ | | outside 5′ |
| | | CTTCATCAG | | ID NO: 146) | | repeat TOP |
| | | (SEQ ID | | | | |
| | | NO: 145) | | | | |
| Cas12a- | HES300 | CTTCCTCAG | CTTC | CTCAGCCGCCGCCG | 23 | fully |
| 9 | | CCGCCGCCG | | CAGGCACAG (SEQ | | outside 3′ |
| | | CAGGCACAG | | ID NO: 148) | | repeat TOP |
| | | (SEQ ID | | | | |
| | | NO: 147) | | | | |
| Cas12a- | HES302 | TCCTCAGCC | TCCT | CAGCCGCCGCCGCA | 23 | fully |
| 10 | | GCCGCCGCA | | GGCACAGCC (SEQ | | outside 3′ |
| | | GGCACAGCC | | ID NO: 150) | | repeat TOP |
| | | (SEQ ID | | | | |
| | | NO: 149) | | | | |

In some embodiments, the Cas protein is present in a base editor, e.g., a cytosine base editor (CBE) or adenine base editor (ABE), e.g., an engineered Cas9 or Cas12 base editor (BE) construct comprising a Cas9 or Cas12 DNA binding domain and a deaminase domain fused at the N or C terminus or inlaid internally, and a uracil DNA glycosylase (UGI) as a fused component or co-expressed (see, e.g., Komor et al., Nature. 2016 May 19; 533 (7603): 420-4; Nishida et al., Science. 2016 Sep. 16; 353 (6305); Kim et al., Nat Biotechnol. 2017 April; 35 (4): 371-376; Komor et al., Sci Adv. 2017 Aug. 30; 3 (8): eaao4774; Gaudelli et al., Nature. 2017 Nov. 23; 551 (7681): 464-471); Jeong et al., Mol Ther. 2020 Sep. 2; 28 (9): 1938-1952; Chu et al., The CRISPR Journal 2021 4:2, 169-177; Carrington et al., Cells. 2020 July; 9 (7): 1690; and Porto et al., Nature Reviews Drug Discovery 19:839-859 (2020)). In some embodiments, the base editor is BE4max or ABEmax, e.g., as described in Koblan et al., Nat. Biotechnol. 2018; 36:843-846 or Komor et al., Sci. Adv. 2017; 3: eaao4774. Preferably the BE comprises a Cas variant as described herein. Also described herein are repeat editors (REds) comprising a Cas9 or Cas12 DNA binding domain and a deaminase domain fused at the N or C terminus or inlaid internally, but no UGI. In some embodiments, the base editor or repeat editor comprises a deaminase domains that has been reported to more efficiently edit cytosines located within a GC sequence context (e.g., evoAPOBEC1, evoCDA, evoFERNY and FERNY)⁶¹.

In some embodiments, the combination of gRNA and Cas is as shown in Table 4.

TABLE 4
Exemplary gRNA/Cas enzyme combinations

| enzyme | gRNA |
|---|---|
| REd-FERNY-nSpG-[no-UGIs] | HES264 |
| REd-CDA1-nSpG-[no-UGIs] | RTW5 |
| REd-CDA1-dSpG(D10A/H840A)-[no-UGIs] | HES285 |
| CBE-CDA1-dSpG(D10A/H840A)-UGI-UGI | HES285 |
| REd-evoCDA1-nSpG-[no-UGIs] | RTW5 |
| REd-CDA1-nSpG-[no-UGIs] | HES283 |
| REd-APOBEC-nSpG-[no-UGIs] | HES264 |
| SpRY nuclease | HES251 |
| enAsCas12a nuclease | RTW8 |
| enAsCas12a nuclease | RTW30 |
| SpCas9 nuclease | HES251 |
| SpRY nuclease | HES252 |
| SpRY nuclease | HES245 |
| SpCas9 nuclease | HES244 |
| SpCas9 nuclease | RTW5 |
| SpG nuclease | HES258 |
| SpG nuclease | HES261 |
| REd-CDA1-dSpG(D10A/H840A)-[no-UGIs] | HES264 |
| REd-CDA1-nSpG-[no-UGIs] | HES264 |
| nSpG(D10A) | HES264 |
| nSpG(D10A) | RTW24 |
| nSpG(H840A) | HES261 |
| REd-CDA1-nSpG-[no-UGIs] | HES284 |
| REd-CDA1-nSpG-[no-UGIs] | RTW5 |
| REd-evoCDA1-nSpG-[no-UGIs] | HES284 |
| REd-evoCDA1-nSpG-[no-UGIs] | HES284 |
| REd-evoCDA1-nSpG-[no-UGIs] | HES283 |
| enAsCas12a nuclease | RTW8 |
| SpG nuclease | HES261 |
| enAsCas12a nuclease | HES294 |
| enAsCas12a nuclease | RTW9 |
| REd-FERNY-nSpG-[no-UGIs] | HES265 |
| REd-APOBEC-nSpG-[no-UGIs] | HES265 |
| nSpG(H840A) | HES264 |
| nSpG(H840A) | HES258 |
| nSpCas9(D10A) | HES253 |
| SpCas9 nuclease | HES253 |
| REd-CDA1-dSpG(D10A/H840A)-[no-UGIs] | HES264 |
| SpCas9 nuclease | HES540 |
| SpCas9 nuclease | RTW24 |
| SpG nuclease | HES264 |
| SpG nuclease | HES285 |
| SpG nuclease | HES286 |
| SpG nuclease | RTW24 |
| SpRY nuclease | HES253 |
| SpRY nuclease | HES538 |
| SpRY nuclease | HES540 |
| nSpCas9(D10A) | RTW24 |
| nSpRY(D10A) | HES252 |
| nSpRY(D10A) | HES253 |
| enAsCas12a nuclease | RTW53 |
| enAsCas12a nuclease | HES296 |
| CBE-CDA-nSpG(D10A)-UGI-UGI | RTW5 |
| REd-CDA-nSpG-[no-UGIs] | RTW5 |
| REd-CDA-dSpG(D10A/H840A)-UGI-UGI | RTW5 |
| REd-CDA-dSpG(D10A/H840A)-[no-UGIs] | RTW5 |
| CBE-CDA-nSpG(D10A)-UGI-UGI | RTW5 |
| REd-CDA-nSpG-[no-UGIs] | RTW5 |
| REd-CDA-dSpG(D10A/H840A)-UGI-UGI | RTW5 |
| REd-CDA-dSpG(D10A/H840A)-[no-UGIs] | RTW5 |
| CBE-CDA-nSpG(D10A)-UGI-UGI | HES265 |
| REd-CDA-nSpG-[no-UGIs] | HES265 |
| REd-CDA-dSpG(D10A/H840A)-UGI-UGI | HES265 |
| REd-CDA-dSpG(D10A/H840A)-[no-UGIs] | HES265 |
| CBE-CDA-nSpG(D10A)-UGI-UGI | HES265 |
| REd-CDA-nSpG-[no-UGIs] | HES265 |
| REd-CDA-dSpG(D10A/H840A)-UGI-UGI | HES265 |
| REd-CDA-dSpG(D10A/H840A)-[no-UGIs] | HES265 |
| CBE-CDA-nSpG(D10A)-UGI-UGI | HES285 |
| REd-CDA-nSpG-[no-UGIs] | HES285 |
| REd-CDA-dSpG(D10A/H840A)-UGI-UGI | HES285 |
| REd-CDA-dSpG(D10A/H840A)-[no-UGIs] | HES285 |
| CBE-CDA-nSpG(D10A)-UGI-UGI | HES285 |
| REd-CDA-nSpG-[no-UGIs] | HES285 |
| REd-CDA-dSpG(D10A/H840A)-UGI-UGI | HES285 |
| REd-CDA-dSpG(D10A/H840A)-[no-UGIs] | HES285 |
| CBE-CDA-nSpG(D10A)-UGI-UGI | HES264 |
| REd-CDA-nSpG-[no-UGIs] | HES264 |
| REd-CDA-dSpG(D10A/H840A)-UGI-UGI | HES264 |
| REd-CDA-dSpG(D10A/H840A)-[no-UGIs] | HES264 |
| CBE-CDA-nSpG(D10A)-UGI-UGI | HES264 |
| REd-CDA-nSpG-[no-UGIs] | HES264 |
| REd-CDA-dSpG(D10A/H840A)-UGI-UGI | HES264 |
| REd-CDA-dSpG(D10A/H840A)-[no-UGIs] | HES264 |
| CBE-CDA-nSpG(D10A)-UGI-UGI | HES283 |
| REd-CDA-nSpG-[no-UGIs] | HES283 |
| REd-CDA-dSpG(D10A/H840A)-UGI-UGI | HES283 |
| REd-CDA-dSpG(D10A/H840A)-[no-UGIs] | HES283 |
| CBE-CDA-nSpG(D10A)-UGI-UGI | HES283 |
| REd-CDA-nSpG-[no-UGIs] | HES283 |
| REd-CDA-dSpG(D10A/H840A)-UGI-UGI | HES283 |
| REd-CDA-dSpG(D10A/H840A)-[no-UGIs] | HES283 |
| CBE-CDA-nSpG(D10A)-UGI-UGI | HES284 |
| REd-CDA-nSpG-[no-UGIs] | HES284 |
| REd-CDA-dSpG(D10A/H840A)-UGI-UGI | HES284 |
| REd-CDA-dSpG(D10A/H840A)-[no-UGIs] | HES284 |
| CBE-CDA-nSpG(D10A)-UGI-UGI | HES284 |
| REd-CDA-nSpG-[no-UGIs] | HES284 |
| REd-CDA-dSpG(D10A/H840A)-UGI-UGI | HES284 |
| REd-CDA-dSpG(D10A/H840A)-[no-UGIs] | HES284 |
| CBE-rAPOBEC1-nSpG(D10A)-UGI-UGI | HES264 |
| REd-rAPOBEC1-nSpG(D10A)-[no-UGI] | HES264 |
| CBE-CDA-nSpG(D10A)-UGI-UGI | HES264 |
| REd-CDA-nSpG(D10A)-[no-UGI] | HES264 |
| CBE-evoAPOBEC-nSpG(D10A)-UGI-UGI | HES264 |
| REd-evoAPOBEC-nSpG(D10A)-[no-UGI] | HES264 |
| CBE-evoCDA-nSpG(D10A)-UGI-UGI | HES264 |
| REd-evoCDA-nSpG(D10A)-[no-UGI] | HES264 |
| CBE-evoFERNY-nSpG(D10A)-UGI-UGI | HES264 |
| REd-evoFERNY-nSpG(D10A)-[no-UGI] | HES264 |
| CBE-FERNY-nSpG(D10A)-UGI-UGI | HES264 |
| REd-FERNY-nSpG(D10A)-[no-UGI] | HES264 |
| CBE-rAPOBEC1-nSpG(D10A)-UGI-UGI | HES284 |
| REd-rAPOBEC1-nSpG(D10A)-[no-UGI] | HES284 |
| CBE-CDA-nSpG(D10A)-UGI-UGI | HES284 |
| REd-CDA-nSpG(D10A)-[no-UGI] | HES284 |
| CBE-evoAPOBEC-nSpG(D10A)-UGI-UGI | HES284 |
| REd-evoAPOBEC-nSpG(D10A)-[no-UGI] | HES284 |
| CBE-evoCDA-nSpG(D10A)-UGI-UGI | HES284 |
| REd-evoCDA-nSpG(D10A)-[no-UGI] | HES284 |
| CBE-evoFERNY-nSpG(D10A)-UGI-UGI | HES284 |
| REd-evoFERNY-nSpG(D10A)-[no-UGI] | HES284 |
| CBE-FERNY-nSpG(D10A)-UGI-UGI | HES284 |
| REd-FERNY-nSpG(D10A)-[no-UGI] | HES284 |
| CBE-rAPOBEC1-nSpG(D10A)-UGI-UGI | RTW5 |
| REd-rAPOBEC1-nSpG(D10A)-[no-UGI] | RTW5 |
| CBE-CDA-nSpG(D10A)-UGI-UGI | RTW5 |
| REd-CDA-nSpG(D10A)-[no-UGI] | RTW5 |
| CBE-evoAPOBEC-nSpG(D10A)-UGI-UGI | RTW5 |
| REd-evoAPOBEC-nSpG(D10A)-[no-UGI] | RTW5 |
| CBE-evoCDA-nSpG(D10A)-UGI-UGI | RTW5 |
| REd-evoCDA-nSpG(D10A)-[no-UGI] | RTW5 |
| CBE-evoFERNY-nSpG(D10A)-UGI-UGI | RTW5 |
| REd-evoFERNY-nSpG(D10A)-[no-UGI] | RTW5 |
| CBE-FERNY-nSpG(D10A)-UGI-UGI | RTW5 |
| REd-FERNY-nSpG(D10A)-[no-UGI] | RTW5 |
| CBE-rAPOBEC1-nSpG(D10A)-UGI-UGI | HES265 |
| REd-rAPOBEC1-nSpG(D10A)-[no-UGI] | HES265 |
| CBE-CDA-nSpG(D10A)-UGI-UGI | HES265 |
| REd-CDA-nSpG(D10A)-[no-UGI] | HES265 |
| CBE-evoAPOBEC-nSpG(D10A)-UGI-UGI | HES265 |
| REd-evoAPOBEC-nSpG(D10A)-[no-UGI] | HES265 |
| CBE-evoCDA-nSpG(D10A)-UGI-UGI | HES265 |
| REd-evoCDA-nSpG(D10A)-[no-UGI] | HES265 |
| CBE-evoFERNY-nSpG(D10A)-UGI-UGI | HES265 |
| REd-evoFERNY-nSpG(D10A)-[no-UGI] | HES265 |
| CBE-FERNY-nSpG(D10A)-UGI-UGI | HES265 |
| REd-FERNY-nSpG(D10A)-[no-UGI] | HES265 |
| CBE-rAPOBEC1-nSpRY(D10A)-UGI-UGI | HES254 |
| CBE-CDA-nSpRY(D10A)-UGI-UGI | HES254 |
| CBE-evoAPOBEC-nSpRY(D10A)-UGI-UGI | HES254 |
| CBE-evoCDA-nSpRY(D10A)-UGI-UGI | HES254 |
| CBE-evoFERNY-nSpRY(D10A)-UGI-UGI | HES254 |
| CBE-FERNY-nSpRY(D10A)-UGI-UGI | HES254 |
| CBE-rAPOBEC1-nSpG(D10A)-UGI-UGI | HES264 |
| CBE-CDA-nSpG(D10A)-UGI-UGI | HES264 |
| CBE-evoAPOBEC-nSpG(D10A)-UGI-UGI | HES264 |
| CBE-evoCDA-nSpG(D10A)-UGI-UGI | HES264 |
| CBE-evoFERNY-nSpG(D10A)-UGI-UGI | HES264 |
| CBE-FERNY-nSpG(D10A)-UGI-UGI | HES264 |
| CBE-rAPOBEC1-nSpG(D10A)-UGI-UGI | HES283 |
| CBE-CDA-nSpG(D10A)-UGI-UGI | HES283 |
| CBE-evoAPOBEC-nSpG(D10A)-UGI-UGI | HES283 |
| CBE-evoCDA-nSpG(D10A)-UGI-UGI | HES283 |
| CBE-evoFERNY-nSpG(D10A)-UGI-UGI | HES283 |
| CBE-FERNY-nSpG(D10A)-UGI-UGI | HES283 |
| CBE-rAPOBEC1-nSpRY(D10A)-UGI-UGI | HES253 |
| CBE-CDA-nSpRY(D10A)-UGI-UGI | HES253 |
| CBE-evoAPOBEC-nSpRY(D10A)-UGI-UGI | HES253 |
| CBE-evoCDA-nSpRY(D10A)-UGI-UGI | HES253 |
| CBE-evoFERNY-nSpRY(D10A)-UGI-UGI | HES253 |
| CBE-FERNY-nSpRY(D10A)-UGI-UGI | HES253 |
| CBE-rAPOBEC1-nSpRY(D10A)-UGI-UGI | HES256 |
| CBE-CDA-nSpRY(D10A)-UGI-UGI | HEES256 |
| CBE-evoAPOBEC-nSpRY(D10A)-UGI-UGI | HES256 |
| CBE-evoCDA-nSpRY(D10A)-UGI-UGI | HES256 |
| CBE-evoFERNY-nSpRY(D10A)-UGI-UGI | HES256 |
| CBE-FERNY-nSpRY(D10A)-UGI-UGI | HES256 |
| enAsCas12a nuclease | HES295 |
| enAsCas12a nuclease | HES298 |
| nSpCas9(D10A) | HES244 |
| nSpCas9(D10A) | HES251 |
| nSpCas9(D10A) | HES540 |
| nSpCas9(D10A) | RTW5 |
| nSpCas9(H840A) | RTW24 |
| nSpCas9(H840A) | HES253 |
| nSpCas9(H840A) | HES244 |
| nSpCas9(H840A) | HES251 |
| nSpCas9(H840A) | HES540 |
| nSpCas9(H840A) | RTW5 |
| nSpG(D10A) | HES258 |
| nSpG(D10A) | HES251 |
| nSpG(D10A) | HES285 |
| nSpG(D10A) | HES286 |
| nSpG(H840A) | HES285 |
| nSpG(H840A) | HES286 |
| nSpG(H840A) | RTW24 |
| nSpRY(D10A) | HES245 |
| nSpRY(D10A) | HES251 |
| nSpRY(D10A) | HES538 |
| nSpRY(D10A) | HES540 |
| nSpRY(H840A) | HES252 |
| nSpRY(H840A) | HES253 |
| nSpRY(H840A) | HES245 |
| nSpRY(H840A) | HES251 |
| nSpRY(H840A) | HES538 |
| nSpRY(H840A) | HES540 |

### Delivery and Expression Systems

The methods can include delivering the Cas variant in a nucleic acid that encodes them. This can be performed in a variety of ways. For example, the nucleic acid encoding the Cas can be delivered as mRNA, or can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the Cas for production of the Cas. The nucleic acid encoding the Cas can also be cloned into an expression vector, for administration to an animal cell, preferably a mammalian cell or a human cell, or to a fungal cell, bacterial cell, or protozoan cell.

To obtain expression, a sequence encoding a Cas is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the Cas is to be administered in vivo for gene regulation, either a constitutive or an inducible promoter can be used, depending on the particular use of the Cas. In addition, a preferred promoter for administration of the Cas can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the Cas, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the Cas, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., lentiviral vectors, adenoviral vectors, SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the Cas guide RNAs or crRNAs can include RNA Pol III promoters, e.g., the H1, U6 or 7SK promoters. These human promoters allow for expression of Cas guide RNAs or crRNAs in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the Cas9.

All of the variants described herein can be rapidly incorporated into existing and widely used vectors, e.g., by simple site-directed mutagenesis. Exemplary plasmids are shown in Table 3.

TABLE 3
Enzyme expression plasmids

| plasmid ID | Cas enzyme | enzyme class | plasmid description |
|---|---|---|---|
| RTW3027 | SpCas9 | nuclease | pCMV-T7-SpCas9-BPNLS-3xFLAG |
| RTW4177 | SpG | nuclease | pCMV-T7-SpG-BPNLS-3xFLAG |
| RTW4830 | SpRY | nuclease | pCMV-T7-SpRY-BPNLS-3xFLAG |
| AAS848 | enAsCas12a | nuclease | pCAG-enAsCas12a-NLS(nuc)-3xHA |
| HES24 | SpCas9 | nickase(D10A) | pCMV-T7-nSpCas9(D10A)-BPNLS- |
| | | | 3xFLAG |
| HES638 | SpG | nickase(D10A) | pCMV-T7-nSpRY(D10A)-BPNLS- |
| | | | 3xFLAG |
| HES636 | SpRY | nickase(D10A) | pCMV-T7-nSpG(D10A)-BPNLS- |
| | | | 3xFLAG |
| RTW3897 | SpCas9 | CBE-BE4max | pCAG-rAPOBEC1-nSpCas9-UGI-UGI |
| HES451 | SpCas9 | CBE-BE4max | pCAG-CDA1-nSpCas9-UGI-UGI |
| HES347 | SpCas9 | CBE-BE4max | pCAG-FERNY-nSpCas9-UGI-UGI |
| HES346/1034 | SpCas9 | CBE-BE4max | pCAG-evoCDA1-nSpCas9-UGI-UGI |
| HES353 | SpCas9 | CBE-BE4max | pCAG-evoFERNY-nSpCas9-UGI-UGI |
| HES610 | SpCas9 | CBE-BE4max | pCAG-evoAPOBEC-nSpCas9-UGI-UGI |
| RTW4552 | SpG | CBE-BE4max | pCAG-rAPOBEC1-nSpG-UGI-UGI |
| HES370 | SpG | CBE-BE4max | pCAG-CDA1-nSpG-UGI-UGI |
| HES310 | SpG | CBE-BE4max | pCAG-FERNY-nSpG-UGI-UGI |
| HES477 | SpG | CBE-BE4max | pCAG-evoCDA1-nSpG-UGI-UGI |
| HES312 | SpG | CBE-BE4max | pCAG-evoFERNY-nSpG-UGI-UGI |
| HES555 | SpG | CBE-BE4max | pCAG-evoAPOBEC1-nSpG-UGI-UGI |
| RTW5133 | SpRY | CBE-BE4max | pCAG-rAPOBEC1-nSpRY-UGI-UGI |
| HES371 | SpRY | CBE-BE4max | pCAG-CDA1-nSpRY-UGI-UGI |
| HES315 | SpRY | CBE-BE4max | pCAG-FERNY-nSpRY-UGI-UGI |
| HES485 | SpRY | CBE-BE4max | pCAG-evoCDA1-nSpRY-UGI-UGI |
| HES358 | SpRY | CBE-BE4max | pCAG-evoFERNY-nSpRY-UGI-UGI |
| HES372 | SpRY | CBE-BE4max | pCAG-evoAPOBEC1-nSpRY-UGI-UGI |
| RTW1028 | enAsCas12a | CBE | pCAG-rAPOBEC1-enAsCas12a-UGI |
| HES1075 | SpG | REd (BE4max no | pCAG-FERNY-nSpG-[no-UGIs] |
| | | UGIs) | |
| HES1082 | SpG | REd (BE4max no | pCAG-evoFERNY-nSpG-[no-UGIs] |
| | | UGIs) | |
| HES1124 | SpG | REd (BE4max no | pCAG-CDA1-nSpG-[no-UGIs] |
| | | UGIs) | |
| HES1135 | SpG | REd (BE4max no | pCAG-evoCDA1-nSpG-[no-UGIs] |
| | | UGIs) | |
| HES1129 | SpG | REd (BE4max no | pCAG-APOBEC-nSpG-[no-UGIs] |
| | | UGIs) | |
| HES1165 | SpG | REd (BE4max no | pCAG-evoAPOBEC-nSpG-[no-UGIs] |
| | | UGIs) | |
| HES1192 | SpG | dCBE | pCAG-CDA1-dSpG(D10A/H840A)- |
| | | | UGI-UGI |
| HES1195 | SpG | dCBE | pCAG-evoCDA1-dSpG(D10A/H840A)- |
| | | | UGI-UGI |
| HES1200 | SpG | dREd (BE4max no | pCAG-CDA1-dSpG(D10A/H840A)- |
| | | UGIs) | [no-UGIs] |
| HES1203 | SpG | dREd (BE4max no | pCAG-evoCDA1-dSpG(D10A/H840A)- |
| | | UGIs) | [no-UGIs] |

Delivery of mRNA or AAV or other viral vectors can also be used; see, e.g., Davis et al., Nature Biomedical Engineering 6:1272-1283 (2022). In some embodiments, the Cas is split into two parts to facilitate delivery in an AAV, e.g., Koblan et al. Nature 589, 608-614 (2021); Villiger et al., Nat. Med. 24, 1519-1525 (2018); Lim et al., Mol. Ther. 28, 1177-1189 (2020); She et al., Sig Transduct Target Ther 8, 57 (2023).

Adeno-associated virus is a naturally occurring defective virus that requires another virus, such as an adenovirus or a herpes virus, as a helper virus for efficient replication and a productive life cycle. (Muzyczka, Curr Top Microbiol Immunol, 158:97-129 (1992)). AAV vectors efficiently transduce various cell types and can produce long-term expression of transgenes in vivo. AAV vectors have been extensively used for gene augmentation or replacement and have shown therapeutic efficacy in a range of animal models as well as in the clinic; see, e.g., Mingozzi and High, Nat Rev Genet, 2011. 12(5): p. 341-55; Deyle and Russell, Curr Opin Mol Ther, 2009. 11(4): p. 442-7; Asokan et al., Mol Ther, 2012. 20(4): p. 699-708). AAV vectors containing as little as 300 base pairs of AAV can be packaged and can produce recombinant protein expression. In some embodiments, the AAV vector can include (or include a sequence encoding) an AAV capsid polypeptide described in PCT/US2014/060163. In some embodiments, the AAV incorporates inverted terminal repeats (ITRs). The AAV can also encode the gRNA, e.g., driven by a promoter known in the art. In some embodiments, a polymerase III promoter, such as a human U6 promoter.

The AAV genomes described above can be packaged into AAV capsids, which capsids can be included in compositions (such as pharmaceutical compositions) and/or administered to subjects. An exemplary pharmaceutical composition comprising an AAV capsid according to this disclosure can include a pharmaceutically acceptable carrier such as balanced saline solution (BSS) and one or more surfactants (e.g., Tween 20) and/or a thermosensitive or reverse-thermosensitive polymer (e.g., pluronic). Other pharmaceutical formulation elements known in the art may also be suitable for use in the compositions described here.

Alternatively, the methods can include delivering the Cas protein and guide RNA together, e.g., as a complex. For example, the Cas and gRNA can be overexpressed in a host cell and purified, then complexed with the guide RNA (e.g., in a test tube) to form a ribonucleoprotein (RNP), and delivered to cells. In some embodiments, the variant Cas can be expressed in and purified from bacteria through the use of bacterial Cas expression plasmids. For example, His-tagged variant Cas proteins can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there's no persistent expression of the nuclease and guide (as you′d get from a plasmid). The RNPs can be delivered to the cells in vivo or in vitro, e.g., using lipid-mediated transfection or electroporation. See, e.g., Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019.

### Compositions

Also described herein are compositions comprising a gRNA and Cas/BE, that can be administered to a subject in need thereof. The compositions can include, e.g., a viral delivery vector, e.g., preferably an adeno-associated virus (AAV) vector that comprises sequences encoding the sgRNA and Cas/BE (as noted above, the BE or Cas can be split, and encoded across two AAV). Alternatively, the compositions can comprise a RNP comprising the Cas/BE complexed with the guide RNA.

Also described herein are pharmaceutical compositions comprising or consisting of a gRNA and Cas/BE, or a nucleic acid encoding the gRNA and Cas/BE, as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88 (2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a kit, container, pack, or dispenser together with instructions for administration.

### EXAMPLES

The invention is further described in the following examples, which do not limit the scope of the invention described in the claims.

### Example 1. Development and Assessment of CRISPR-Based Technologies to Contract HD Genetic Repeats

Here we sought to develop numerous orthogonal gene-editing approaches to permanently reduce or fully eliminate the HD CAG-repeat. We envisioned that these experiments would provide molecular insight into nuclease-induced repeat contraction as a strategy to treat trinucleotide repeat diseases, and may establish a path toward the development of novel HD therapeutics. Published and unpublished CRISPR nucleases that can target sites previously inaccessible with wild-type nucleases can be employed to test the hypothesis that editing events within the CAG-repeat can initiate sequence contraction. Because MMEJ-mediated repair of DSBs can induce variable length triplet contractions via end-resection of broken DNA ends (with imperfect template switching during repair), the original ‘in-phase’ target sites should be restored to re-establish bona fide on-target sites for subsequent re-targeting (thus initiating a cycle of targeting and contraction; see FIG. 1c). We hypothesize that contractions of one or more CAG triplets per event will occur in-frame, since MMEJ-driven DNA repair events have recently been shown to be enriched compared to NHEJ-based indels⁴². Continuous re-targeting of the HTT CAG-repeat will result in progressively shorter poly-Q tracts and the genetic elimination of mHTT protein production (FIG. 1c). The CRISPR nuclease target sites will only be destroyed when the CAG-repeat is fully or nearly fully contracted. While the proposed strategies cannot differentiate the HTT allele from the repeat expanded mHTT allele per se, if the deletions occur in-frame, they should generate functional N-terminally truncated alleles that should in principle reduce the risk or delay the onset of HD. Experiments can be conducted with human HEK293T cells due to ease of transfection and reagent testing, with subsequent validation of top candidate strategies in humanized murine cell lines with expanded CAG-repeat sizes⁴³⁻⁴⁵, with eventual translation of optimal strategies in HD-patient derived iPS cells.

### Methods

The following materials and Methods were used in this Example.

### Target Sites, Plasmids, and Oligonucleotides

The gRNA sequences and target sites used for Cas9 and Cas12a enzymes are listed in Tables 1 and 2, respectively. Expression plasmids for human U6 promoter-driven SpCas9, SpG, or SpRY sgRNAs were generated by annealing and ligating duplexed oligonucleotides corresponding to spacer sequences into BsmBI-digested pUC19-U6-BsmBI_cassette-SpCas9_sgRNA (BPK1520; Addgene plasmid 65777). Expression plasmids for human U6 promoter-driven enAsCas12a crRNAs were generated by annealing and ligating duplexed oligonucleotides corresponding spacer sequences into BsmBI-digested pUC19-BsmBI_cassette-AsCas12a_crRNA (BPK3079; Addgene plasmid 78741). Plasmids encoding various nucleases, nickases, base editors, and repeat editors were cloned via isothermal assembly⁶⁸ (and are listed in Table 3).

### Cell Culture and Transfections

Human HEK 293T cells (American Type Culture Collection; ATCC) were cultured in Dulbecco's Modified Eagle Medium (DMEM) supplemented with 10% heat-inactivated FBS (HI-FBS) and 1% penicillin-streptomycin. Samples of supernatant media from cell culture experiments were analyzed monthly for the presence of mycoplasma using MycoAlert PLUS (Lonza).

HEK 293T human cell transfections were performed 20 hours following seeding of 2×10⁴ HEK 293T cells per well in 96-well plates. Transfections for nucleases or nickases contained 29 ng of enzyme expression plasmid and 12 ng of gRNA or crRNA expression plasmid mixed with 0.3 μL of TransIT-X2 (Mirus) in a total volume of 15 μL Opti-MEM (Thermo Fisher Scientific). Transfections for base editors or repeat editors contained 70 ng of enzyme expression plasmid and 30 ng gRNA expression plasmid mixed with 0.72 μL of TransIT-X2 in a total volume of 15 μL Opti-MEM. Transfection mixtures were incubated for 15 minutes at room temperature and then distributed across the seeded HEK 293T cells. Experiments were halted after 72 hours and genomic DNA (gDNA) was collected by discarding the media, resuspending the cells in 100 μL of quick lysis buffer (20 mM Hepes pH 7.5, 100 mM KCl, 5 mM MgCl₂, 5% glycerol, 25 mM DTT, 0.1% Triton X-100, and 60 ng/μL Proteinase K (New England Biolabs; NEB)), heating the lysate for 6 minutes at 65° C., heating at 98° C. for 2 minutes, and then storing at −20° C.

### Assessment of Editing Efficiency

The efficiency of genome modification was determined by next-generation sequencing (NGS) using a 2-step PCR-based Illumina library construction method, similar to as previously described³¹. Briefly, genomic loci were amplified using approximately 50-100 ng of gDNA, Q5 High-fidelity DNA Polymerase (NEB), and PCR-1 primers. The gene specific regions of the PCR-1 primers used to amplify HTT exon 1 were oHES226 (forward1-GGGAGACCGCCATGGCGAC (SEQ ID NO: 151)), oHES229 (reverse1-GGCTGAGGCAGCAGCGGCTG (SEQ ID NO: 152)), oHES163 (foward2-CATGGCGACCCTGGAAAAGCTGATG (SEQ ID NO: 153)), and oHES168 (reverse2-CTGAGGAAGCTGAGGAGGCGG (SEQ ID NO: 154)).

Cycling conditions of PCR-1 were 1 cycle at 98° C. for 2 min; 35 cycles of 98° C. for 10 sec, 58° C. for 10 sec, 72° C. for 20 sec; and 1 cycle of 72° C. for 1 min. PCR products were purified using paramagnetic beads prepared as previously described^{41,69}. Approximately 20 ng of purified PCR-1 products were used as template for a second round of PCR (PCR-2) to add barcodes and Illumina adapter sequences using Q5 and primers (using PCR-2 primers as previously described³¹) and cycling conditions of 1 cycle at 98° C. for 2 min; 10 cycles at 98° C. for 10 sec, 65° C. for 30 sec, 72° C. 30 sec; and 1 cycle at 72° C. for 5 min. PCR products were purified prior to quantification via capillary electrophoresis (Qiagen QIAxcel), normalization, and pooling. Final libraries were quantified by qPCR using the KAPA Library Quantification Kit (Complete kit; Universal) (Roche) and sequenced on a MiSeq sequencer using a 300-cycle v2 kit (Illumina). The resulting data was analyzed using CRISPResso2⁵², optionally with modifications to report repeat-related metrics (for repeat contraction, allele length, etc.)

### Example 1.1: CRISPR-Cas9 Nucleases and Nickases to Induce CAG-Repeat Contraction

In contrast to the formerly prevailing belief that SpCas9-induced breaks in DNA resulted in variable length indels (often referred to as ‘random indels’), recent characterization of the molecular products of Cas9 edits revealed that DSBs outcomes predominantly result in high levels of sequence-dictated deletions, with fewer insertions^{42,46-48}. Additionally, these studies suggested that the size of the deletion is heavily dependent on the sequence surrounding the DNA break, often resulting predicable editing ‘fingerprints’ that are specific to individual target sites. The likelihood of defined deletions occurring is further enhanced when there is homology between the sequences on opposite sides of the break, since strand resection and transient pairing between the two broken ends can result in MMEJ repair⁴⁶ (FIG. 1a). Since the CAG-repeat is highly repetitive, we hypothesized that the CAG-repeat should be inherently prone to MMEJ-based in-frame reductions of CAG codons when a DNA break is targeted within the repeat (FIG. 1c). Positioning DNA DSBs or nicks within the repetitive element therefore has the potential to precisely contract the repeat.

We sought to investigate the potential of highly targetable SpCas9 nucleases and nickases to reduce the HTT CAG-repeat to sub-pathogenic levels. To do so, we will utilize our recently described SpCas9 variants³¹ capable of targeting alternate PAMs that will enable higher resolution targeting of the sequences flanking and within the CAG-repeat. Since there are no NGG PAMs that enable positioning a DNA break or nick within the CAG repeat with wild-type SpCas9, our SpCas9 variants that can recognize NGN and NAN PAMs (where N is any DNA base) are required for targeting the mHTT sequence. These variants increase the targeting range of SpCas9 by more than 10-fold, providing additional sites for targeting either the 5′ or 3′ junction of the CAG-repeat. Furthermore, these variants enable us to interrogate formerly inaccessible targets sites positioned near or across the exon1-CAG junction to varying phases, enabling us to determine the optimal positioning of DSBs or nicks in different regions of the repeat.

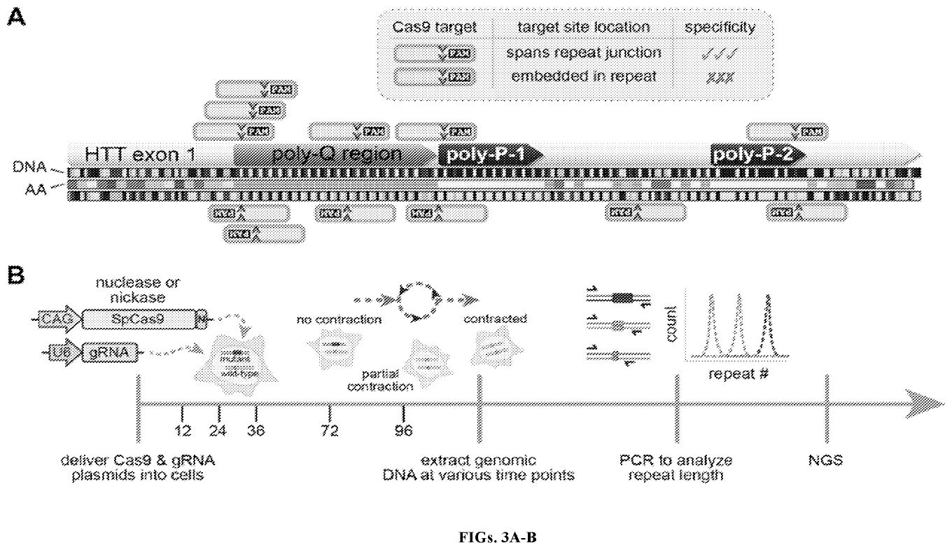

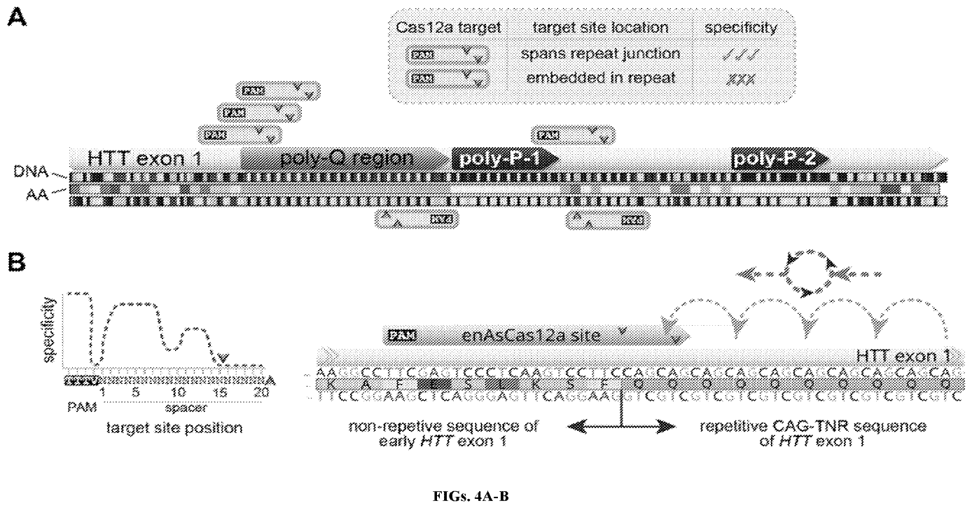

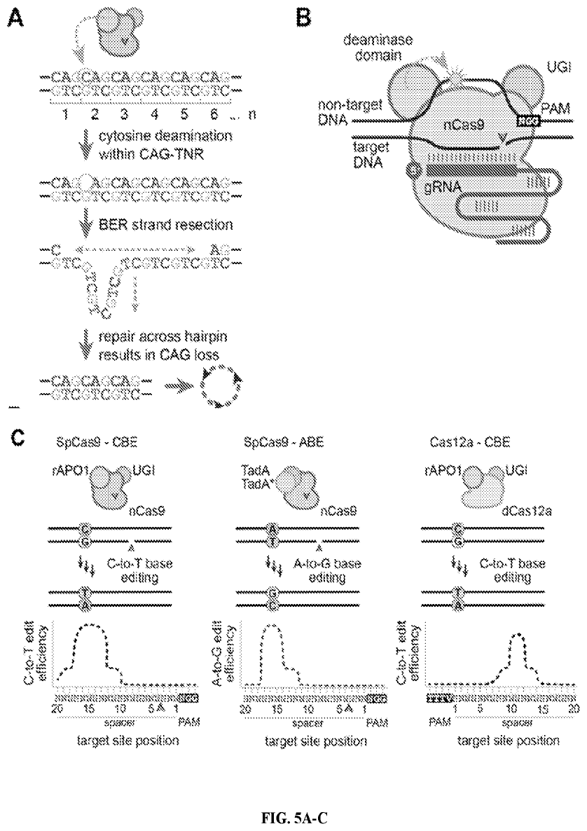

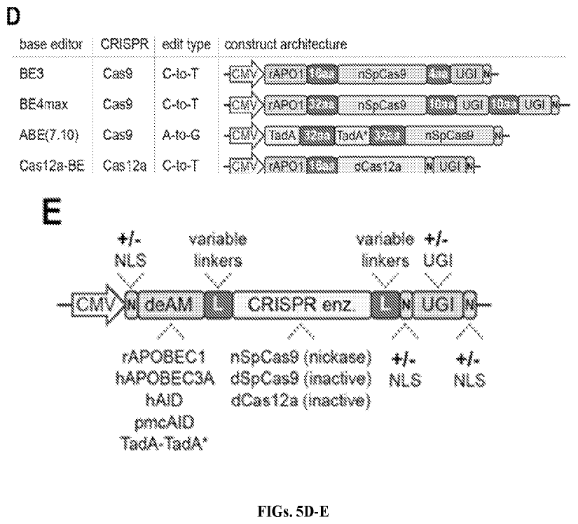

To initiate efficient repeat contraction, we hypothesized that a DNA break must occur within or immediately proximal to the trinucleotide repeat sequence. Since SpCas9 generates a DSB internally within its target site (see FIG. 2b), a large portion of the SpCas9 target site must lie within the CAG (Glutamine) or CCG (Proline) repeats for the DNA break to initiate sequence contraction of HTT exon 1 (FIG. 3a). Conversely, positioning the target site too far into the CAG-repeat risks potential off-target effects due to loss of target specificity, since CAG repeat sequences are found frequently throughout the human genome. To enable targeting sites near the repeat boundary, we utilize SpCas9 variants that can target PAMs of the forms NGN (NGA, NGC, NGG, and NGT) and NAN (FIG. 2e). These variants increase targeting density at the junction of the exon 1 and CAG-repeat sequences without necessitating targeting wholly within the repeat, a benefit to prevent undesirable DSBs within other genomic CAG sequences.

To utilize our previously engineered and our recently developed set of SpCas9 variants^{31,32}, we designed a comprehensive list of guide RNAs (gRNAs) that would permit high resolution nicking, cleavage, or deamination within various phases of the CAG repeat (Table 1). We annotated all possible NGA, NGC, NGG, NGT (collectively NGN), NAN, NCN, and NYN target sites near the CAG-repeat. Target site design was focused on three regions of HTT exon 1, searching for PAMs that enable target sites to straddle the: (1) exon1-polyQ junction, (2) the polyQ and poly-proline (polyP) junction, and (3) the polyP-exon1 junction (FIG. 3a). In particular, our NGC PAM variants will dramatically increase the number of target sites that flank the exon1-CAG or CAG-exon1 junctions, since there are many NGC PAMs in the CAG-CAG and CTG-CTG repeats encoded on the coding and non-coding strands, respectively; FIG. 3a). There are also target sites with NGA, NGT, NAN, NCN, and NYN PAMs that span these same HTT regions.

Since MMEJ-based repair of the DSB should reconstitute the original target site (if the CAG-repeat is contracted in-frame), subsequent re-targeting by SpCas9 will lead to further contraction of the repeat (FIGS. 1c). Thus, the repeated cycle of targeting the mHTT CAG-repeat, repair/restoration, and re-targeting, should eventually lead to the genetic elimination of the CAG-repeat. The CRISPR nuclease target sites should in principle only be eliminated when the CAG-repeat is fully contracted, or if repair occurs out-of-frame. We tested the efficacy of this approach using SpCas9 PAM variants and corresponding gRNAs that tile the exon1-polyQ, polyQ-polyP, and polyP-exon1 junctions (FIG. 3a).

In addition to SpCas9 nucleases, we also sought to evaluate the ability of SpCas9 nickases to contract the CAG repeat sequence. DNA nicks are known to engage different repair factors compared to DSB, and models have been proposed for efficient excision of repetitive regions during gap repair (resulting from collapse of ssDNA as a result of resected DNA ends). This hypothesis was previously examined with SpCas9 (ref.³⁰), but repeat contraction was inefficient because target sites known to be weakly targeted by wild-type SpCas9 with non-canonical NAG PAMs were utilized due to the lack of available sites encoding canonical NGG PAMs. To evaluate the effectiveness of SpCas9 nickases at contracting repetitive sequences, we separately generated the D10A (RuvC inactivating) or H840A (HNH inactivating) nickase versions of the SpCas9 PAM variants that nick the non-target and target strands, respectively^{49,50}. Depending on the target site orientation, the D10A and H840A nickases will generate a lesion on the transcribed or non-transcribed strands of the HTT gene. Nicks positioned on either strand may potentially result in different repair outcomes due to collisions with RNA polymerase and interactions with other DNA repair factors⁵¹.

To assess the activities of various Cas9 nucleases and nickases, we first cloned the gRNAs. Oligonucleotides corresponding to the spacer sequences of the gRNAs for various target sites were cloned into an SpCas9 sgRNA expression vector (Table 1), and plasmids were verified by Sanger sequencing. The activities of various SpCas9 enzymes and gRNAs were then assessed via transfection into HEK293T cells (using enzyme and gRNA expression vectors), genomic DNA was extracted 3 days following transfection, PCRs of the targeted region were performed, and we sequenced the edited products by Illumina sequencing with analysis using CRISPResso2 (ref.⁵²) (FIG. 3B).

### Example 1.2: HTT Editing Using Various Cas9 Nucleases

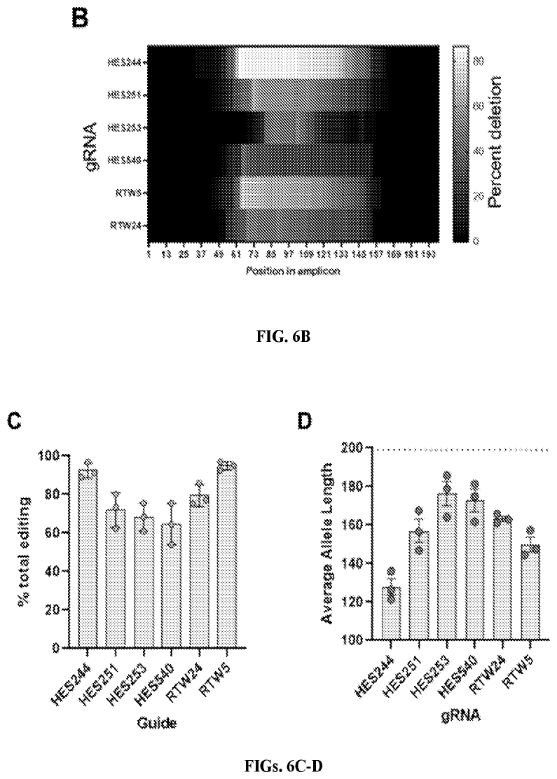

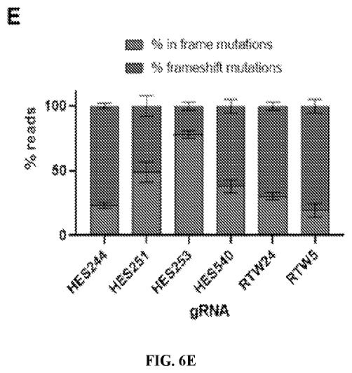

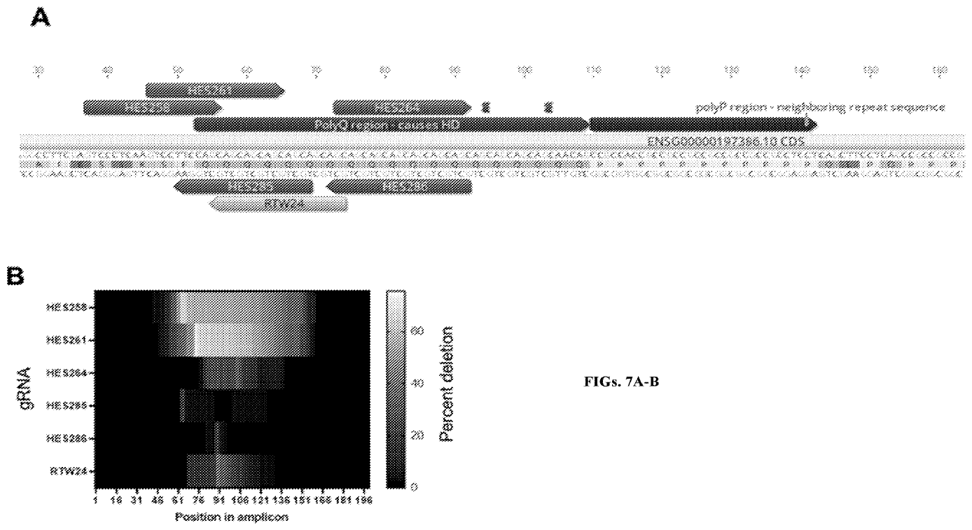

We initially assessed the on-target editing efficiency of wild-type (WT) SpCas9 targeting sites with NGG PAMS near the CAG repeat, or sites with non-canonical NAG PAMs within the repeat (FIG. 6a; Table 1). Plasmids encoding WT SpCas9 and the gRNAs were transfected into HEK 293T cells, genomic DNA was extracted 72 hours later, and PCRs were performed for next-generation sequencing. Data analysis revealed that some gRNAs led to efficient large deletions within the polyQ repeat (FIG. 6b; see gRNA HES253). Furthermore, other gRNAs led to deletions across the polyQ and polyp regions. Most deletions were anchored near the polyQ or polyp boundaries (FIG. 6b). Since these sites were targeted efficiently by WT SpCas9, we observed very high levels of editing (sometimes nearing 100%; FIG. 6c) and high degrees of repeat contraction (FIG. 6d). If the deletion mutations were random, one would anticipate that ⅓ (33%) of the alleles would be in-frame and ⅔ would be out-of-frame. Depending on the gRNA used, some edit profiles were substantially higher than 33% in-frame, suggesting that carefully selecting combinations of Cas enzymes and gRNAs might lead to higher proportions of in-frame contraction of the CAG repeat (FIG. 6e).

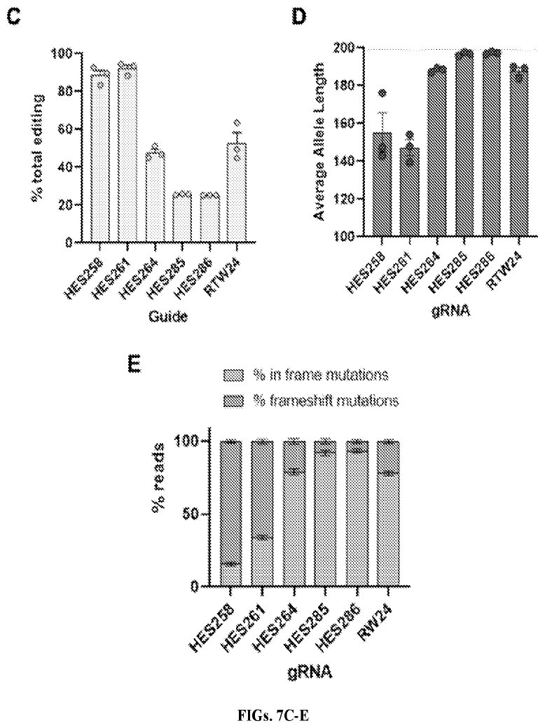

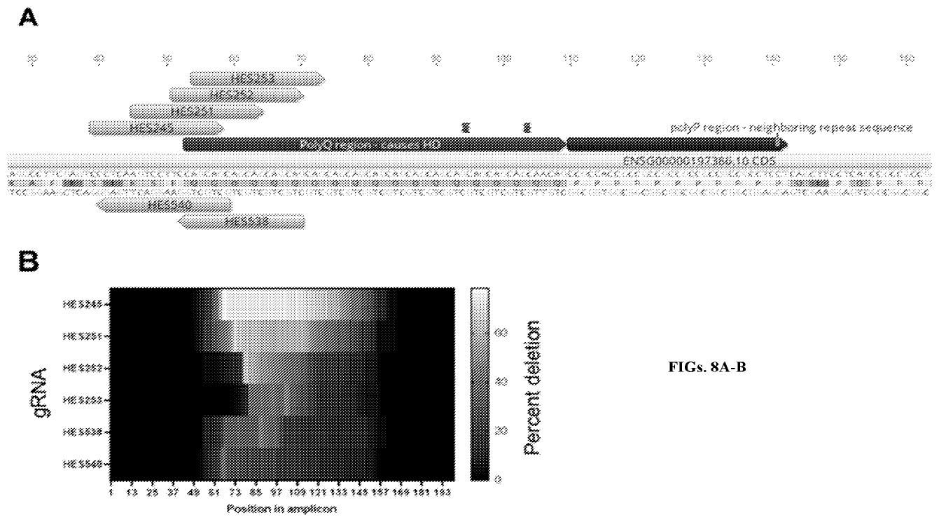

Next, we performed analogous experiments using our previously described SpG nuclease³¹ that can target an expanded range of NGN PAMs (avoiding the ‘G’ requirement in the 3^{rd} position of the PAM, which is required for WT SpCas9). The use of SpG enables more flexibility to design and utilize gRNAs more precisely positioned around the boundary of 5′ exon 1 and the 5′ end of the CAG repeat. We selected six different gRNAs targeted to sites with NGN PAMs, either fully within the repeat on the top or bottom strand, or on the boundary of the 5′ end of the CAG repeat (FIG. 7a). We performed transfections with plasmids encoding SpG nuclease and the gRNAs, extracted genomic DNA 72 hours later, and performed PCRs for next-generation sequencing. We observed a range of deletion profiles (FIG. 7b), overall editing efficiencies (FIG. 7c), average allele lengths (FIG. 7d), and in-frame edits (FIG. 7e) depending on the gRNA used. Although the editing was lower in efficiency with some gRNAs, with SpG they appeared to lead to higher proportions of in-frame contractions, in many cases well above the 33% threshold of random deletion profiles.

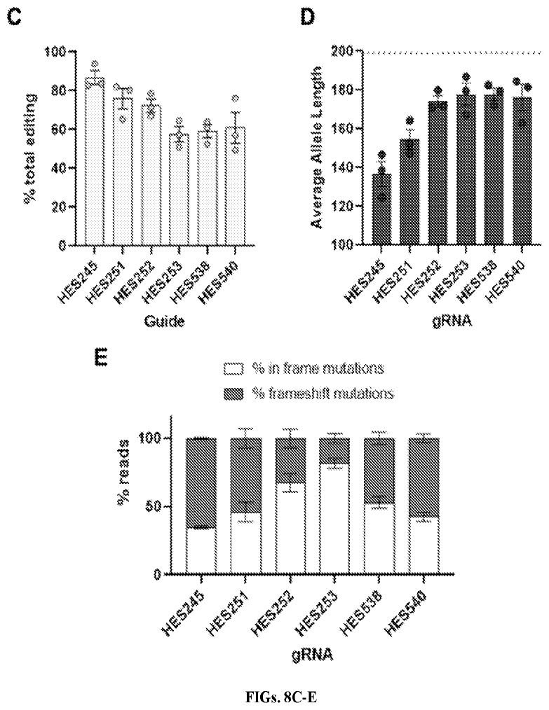

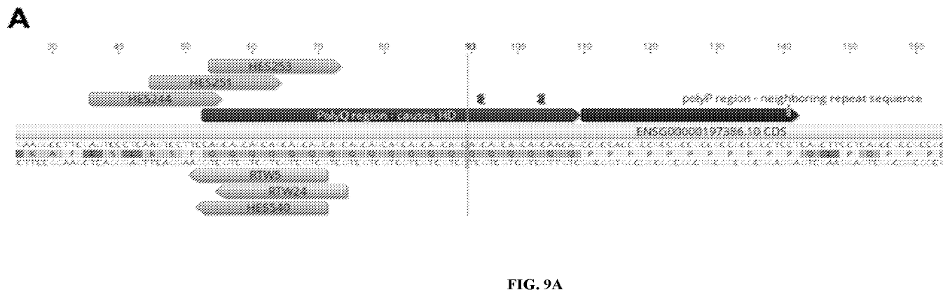

Finally, we utilized SpRY in nuclease-based transfections in HEK 293T cells. SpRY is an enzyme that we previously engineered to relax its PAM requirement³¹, permitting targeting of sites with NRN PAMs and sometimes sites with NYN PAMs. We performed transfections using 6 gRNAs in different phases of the CAG repeat or flanking the exon/5′ repeat boundary (FIG. 8a). With all gRNAs, we observed medium-to-high levels of deletions within the CAG repeat (FIG. 8b) and overall editing (FIG. 8c). We also observed a range of mean contraction lengths (FIG. 8d) and in-frame mutations alleles (FIG. 8e), with gRNA 252 being the most promising in terms of efficiency, deletion capacity, and in-frame alleles (approximately 66% of edits were in-frame).

Together, these results reveal that potent nuclease-based editing of the CAG repeat is possible when using WT SpCas9 or engineered PAM variants SpG and SpRY. Depending on the gRNA used, it's possible to titrate the breadth of deletion alleles, the overall editing efficiency, and also the proportion of in-frame alleles.

### Example 1.3: HTT Editing Using Cas9 Nickases

We evaluated the effectiveness of various SpCas9 nickases at modifying and contracting the HTT CAG repeat. We generated the D10A and H840A nickase versions of WT SpCas9 and SpG (the latter of which targets NGN PAMs), and assessed their ability to perturb the repeat using a series of gRNAs. Target modification activities determined by transfection of expression plasmids into human cells, modification was assessed by targeted next-generation sequencing (NGS), and data were analyzed to determine overall editing efficiencies.

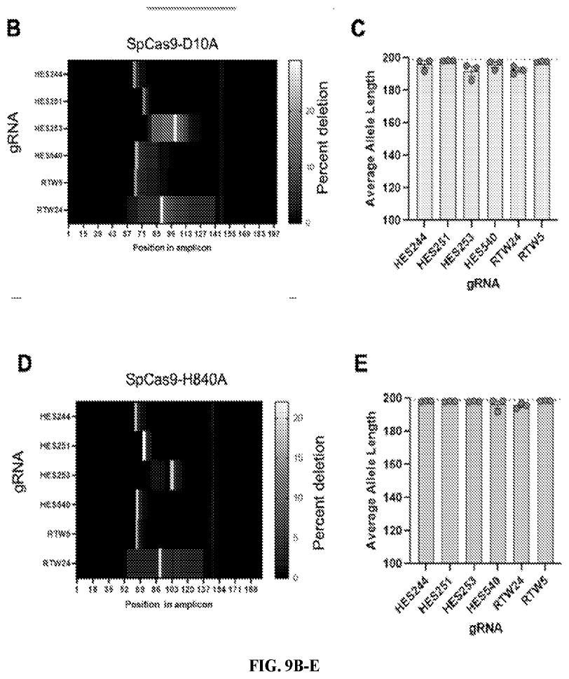

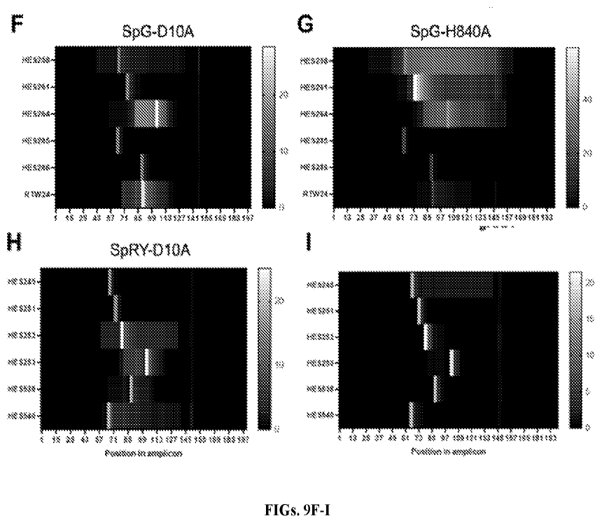

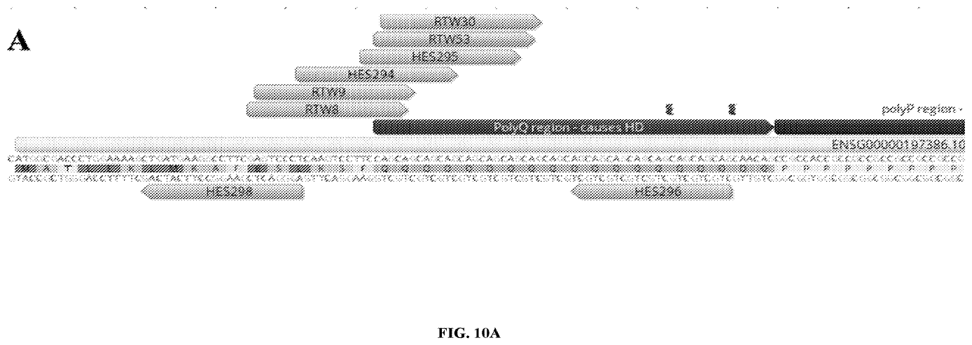

We performed transfections using both types of SpCas9 nickases, the D10A and H840A nickases, which inactivate the RuvC or HNH catalytic domains, respectively. Initially we performed experiments using WT SpCas9-based nickases and the six gRNAs that we previously utilized for the nuclease-based transfections (FIG. 9a). With the SpCas9-D10A nickase, we observed only low levels of deletions (FIG. 9b) or alteration in allele length (FIG. 9c). Notably, the HES253 gRNA that displayed the greatest degree of editing with with SpCas9-D10A is fully embedded within the CAG repeat (targeting repeated sites with NAG PAMs), and is the same gRNA utilized previously by Cinesi et al.³⁰. We also explored the use of the SpCas9-H840A nickase using the same six gRNAs. We observed only low levels of deletions/editing or alterations in allele length (FIGS. 9d and 9e).

Next, we performed experiments using nickases derived from the SpG and SpRY enzymes, given their ability to target different phases of the repeat. For SpG we used gRNAs targeting NGN PAMs (FIG. 7a) and for SpRY we used gRNAs targeting NRN PAMs (FIG. 8a). With the D10A and H840A versions of SpG, we observed reasonable (20-40%) contraction in some cases depending on the gRNA (FIGS. 9f and 9g). Although the overall editing was lower than what we observed with the nuclease equivalent experiments, the percentage of alleles that remained in-frame was high. As with SpCas9, some gRNAs with the SpG-D10A or H840A nickases exhibited low-to-no evidence of editing. Finally, we performed experiments using SpRY-D10A and SpRY-H840A constructs (FIGS. 9h and 9i). We observed low levels of editing with SpRY-D10A and some gRNAs, but the majority of SpRY-D10 or SpRY-H840A conditions led to near-background levels of editing or repeat contraction.

Together, these results demonstrate that although some nickase/gRNA pairs can lead to appreciable levels of editing and repeat contraction (often in-frame), the overall levels of CAG perturbation are much less efficient than when performing experiments with the comparable nuclease constructs.

### Example 1.4: CRISPR-Cas12a Nucleases to Contract the HTT CAG-Repeat

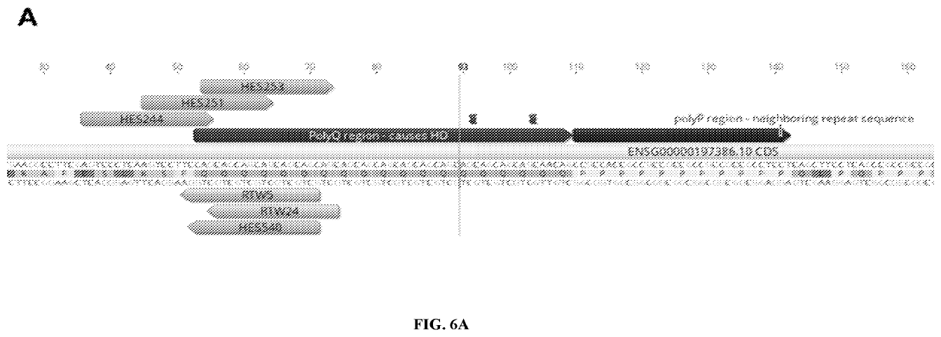

The expanding toolbox of different CRISPR nucleases provides additional genome editing technologies that have beneficial properties compared to the prototypical SpCas9. Here we took advantage of distinct characteristics of Cas12a nucleases (formerly called Cpf1³⁶) to contract the CAG-repeat. Compared to SpCas9, Cas12a nucleases possess several distinct properties that include: recognition of an extended T-rich PAM, catalysis that generates 5′-overhangs (compared to a blunt DSB by SpCas9), the initiation of DSBs at the very PAM distal end of the Cas12a target site (compared to PAM proximal breaks with SpCas9), the ability to process individual crRNAs out of a single transcript to enable multiplex targeting, and the requirement for only a single short ~40 nt crRNA compared to the 100 nt sgRNA for SpCas9 (FIG. 2b). We previously determined that AsCas12a can robustly function for genome editing in human cells and that it possesses high genome-wide specificity³⁵. Cas12a nucleases generally lack specificity in their target site near where the DSB occurs, and thus can tolerate small indels in the PAM distal end of their target sites (FIG. 2b). This property contrasts with SpCas9, where indels within its target site overlapping the cleavage site may disrupt binding and prevent subsequent DSB events (leading to insufficient repeat contraction). We therefore hypothesized that AsCas12a target sites situated on the boundary of the CAG-repeat may offer advantages for efficient contraction due to persistent cleavage until the Cas12a target site is eventually destroyed, presumably after the CAG-repeat has been fully reduced (FIG. 4b). Furthermore, because of AsCas12a's exquisite genome-wide specificity, we anticipate that target sites that span the exon1-CAG junction may prevent genome-wide targeting of CAG sequences. However, one major obstacle that precludes the use of wild-type AsCas12a for targeting the GC-rich CAG-repeat is that there are no available TTTV PAMs (where Vis any nucleotide except for T) for wild-type AsCas12a within or proximal to the repeat. To overcome this targeting range limitation and to leverage the desirable properties of AsCas12a, we utilized our recently described enAsCas12a variant that can target previously inaccessible PAMs⁴¹. The enAsCas12a variant also possesses 2-to-3-fold increased on-target activities, improving the likelihood that the variant can induce efficient contraction of the HTT-repeat. We hypothesized that several advantageous characteristics of enAsCas12a will enable robust and efficient contraction of the CAG-repeat without collateral genome-wide effects, including: (1) improved on-target activity, (2) tolerance of indels within the DSBs portion of the target site, enabling successive rounds or targeting and re-cleavage, and (3) high genome-wide specificity.

To assess the ability of enAsCas12a to target and contract the CAG-repeat, we identified, designed, and cloned crRNAs (or also called guide RNAs; gRNAs) corresponding to putative enAsCas12a target sites that span the exon1-polyQ, the polyQ-polyP, and the polyP-exon1 junctions (FIG. 4a and Table 2). Because wild-type Cas12a enzymes can typically only target sites that encode TTTV PAMs, there are no canonical target sites for wild-type Cas12a nucleases that can be utilized within these regions of HTT. However, enAsCas 12 has a substantially expanded targeting range that enables recognition of sites harboring PAMs that include TTTN, NTTV, TTCN, TRTV, TCCV, and others. This expanded targeting range will in principle enable the design and targeting of several new sites within the CAG-repeat, all previously inaccessible with wild-type Cas 12a nucleases.

To determine whether Cas12a nucleases could contract the CAG-repeat, HEK293T cells were transfected with enAsCas 12a nuclease (Table 3) and crRNA expression vectors (Table 2), and genomic DNA was extracted 3 days following transfection. To determine modification efficiency, aliquots of genomic DNA were used as templates for PCR of the targeted regions and edited products were quantified by Illumina next-generation sequencing (NGS). Data was analyzed using our modified version of CRISPResso2 to permit quantification of repeat-relevant data (e.g. percentage of in-frame alleles, histograms of the distributions of repeat lengths, total CAG repeat editing, etc.).

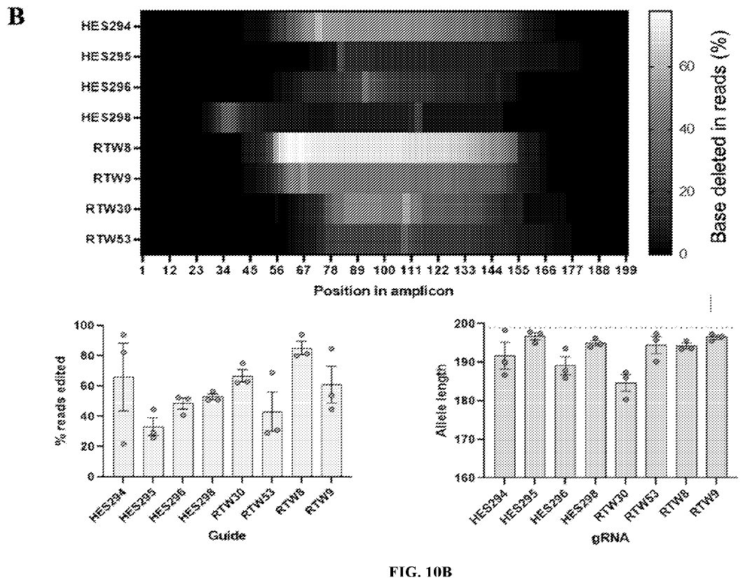

We assessed the on-target efficiency of enAsCas12a against various target sites in HTT exon 1. Transfections were performed using 8 different crRNAs targeted to various target sites harboring enAsCas12a-accessible PAMs (FIG. 10A). For each of the crRNAs, we plotted a vidualization of the percent deletion of each base in the amplicon, the overall editing percentage within the population of reads, and the average allele length (FIG. 10B). In some cases, we observed substantial levels of editing at or within the repeat. For example, crRNA-RTW8 led to very high levels of editing and wide contraction of the CAG repeat (and that also extended into the neighboring poly-proline region; FIG. 10B). An additional crRNA-RTW30 also led to high levels of editing, but that was more constrained to within the repeat and the resulting alleles were largely in-frame (FIG. 10B). The control crRNA HES298 was designed to target outside the CAG repeat, which led only to indels early in HTT exon 1 without any meaningful perturbation of the repeat (FIG. 10B).

Together, these results demonstrate the potential of enAsCas12a to generate large sequence perturbations to the CAG repeat, and in some cases also the flanking sequence.

### Example 1.5: CRISPR Base Editors and Repeat Editors to Alter or Contract CAG-Repeats

A potential approach to reduce genetic repeat length would be to engage a DNA repair pathway called base-excision repair (BER), by specifically deaminating DNA bases within the CAG-repeat to induce fragility and reduction of the repeat. Several studies have long indicated the pivotal contributions of different DNA repair processes to repeat stability, where misregulation or intentional suppression of proteins involved in DNA repair can lead to repeat expansions or contractions⁵³⁻⁵⁷. It was recently demonstrated in Saccharomyces cerevisiae that by modifying R-loop stability (RNA: DNA hybrids that occur during transcription), repetitive sequences can become volatile and contract⁵⁸. The authors hypothesized that persistent accessibility of the displaced non-template DNA strand during stalled transcription events can lead to extended contact with endogenous deaminases. Cytosine deamination events on the displaced strand engage BER to repair the deaminated bases, leading to long-range strand resection. During BER, the repetitive sequences on the opposite intact strand (such as the complementary CTG-strand to the CAG-repeat) are prone to the formation of energetically favorable hairpins, resulting in repeat sequence loss when the BER-undergoing repeat-containing non-template strand is repaired using the hairpin-containing DNA as a template⁵⁹ (similar to as shown in FIG. 5a). Together, this and other work reinforces the notion that different DNA repair process may be exploited to address the CAG-repeat of HD.

To directly determine whether targeted deamination of the CAG-repeat can induce contraction, we sought to utilize and engineer CRISPR base editor (BE) or repeat editor (REd) technologies that direct BER events within the repeat (FIG. 5a). BEs are fusion proteins comprised of a catalytically inactive or nickase version of SpCas9 (dCas9 or nCas9, respectively) fused to deaminase domains, including APOBEC, AID, and others, as well as a uracil glycosylase inhibitor (UGI) to maintain the identity of the edited base by subverting DNA repair^{20-22,60,61} (FIGS. 5b and 5c). When the gRNA of the BE complex is paired with the target DNA strand, the non-target strand becomes solvent exposed and is a target for deamination by the tethered deaminase (FIG. 5b). Whereas cytosine base editors (CBEs) enable the direct conversion of cytosine to thymine (C-to-T), adenine base editors (ABEs) were also recently described that catalyze the conversion of adenine to guanine⁶²⁻⁶⁴ (A-to-G). Several studies have demonstrated highly-efficient C-to-T or A-to-G editing in many different species^{16,22,65}, yet one major caveat of both CBEs and ABEs is that potent editing is only possible in short sequence windows within the target site (FIG. 5c). Thus, like CRISPR nucleases, efficient editing with BEs is dependent on the availability of PAMs to appropriately position the base editing event. To edit the CAG repeat, we engineered BE fusions that have expanded targeting ranges to specifically deaminate C or A bases within the CAG-repeat, and will also optimize SpCas9- and Cas12a-based BEs more prone to initiating BER to contract the CAG repeat.

While BE technologies have broadly enabled the installation of C-to-T and A-to-G substitutions, there are several properties of SpCas9-BEs that may be suboptimal for contraction of the CAG-repeat. First, common forms of SpCas9-BEs (called BE3 (ref.²⁰) and BE4max⁶⁶; see FIG. 5d) utilize a fused uracil glycosylate inhibitor (UGI) to enhance the intended C-to-T edit by preventing endogenous BER from re-installing the original cytosine in place of the deaminated base. Because in our present study we may want to promote rather than inhibit BER, BE fusions that include the UGI domain could potentially reduce the potency of CAG contraction. Thus, we engineered new versions of BEs called repeat editors (REds) without the fused UGI or other domain alterations (including making a catalytically inactive dCas9 that harbors an H840A mutation in addition to the conventional D10A RuvC-inactivating nCas9 mutation in BEs) to determine which are more likely to induce repeat contraction (FIG. 5e). A second major issue of canonical SpCas9-BEs is their targeting range restriction to sites that encode NGG PAMs, preventing broad targeting within the CAG-repeat. To circumvent these two potential issues, we: (1) engineered versions of SpCas9-CBEs+/−UGI, (2) optimized the BE architecture by generating fusions of different deaminase domains (APOBEC1, APOBEC3A, AID, etc.) to either the N- or C-terminus of dead Cas9 (dCas9) or nickase Cas9 (nCas9), and (3) utilized our new SpG and SpRY variants capable of targeting non-canonical NGN and NAN PAMs to better position the BE or REd ‘edit window’ within the CAG-repeat.

In addition to SpCas9 BEs, Cas12a-based base-editors (Cas12-BEs) have also been described^{41,67}, offering the ability to target novel sequences for C-to-T editing. However, as detailed above, the restricted targeting range of the original Cas12a-BE constructs are not compatible with targeting within the CAG-repeat due to lack of available canonical TTTV PAMs. To overcome this restriction for Cas12a base editing applications, we recently described enhanced AsCas12a-BEs (enAsCas12a-BEs) that have greatly expanded targeting range (including many PAMs that will enable deamination in the repeat) and several fold-improved C-to-T editing activity⁴¹. Thus, BER-prone enAsCas12a-BEs can be generated by varying the presence or absence of UGI, and by altering the identity and position of deaminase domain fusions (similar to as described above for SpCas9; see FIGS. 5c, 5d, and 5e). Together, the collection of engineered SpCas9- and enAsCas12a-BEs and REds will offer us several technologies capable of initiating BER within the CAG-repeat to induce repeat contraction.

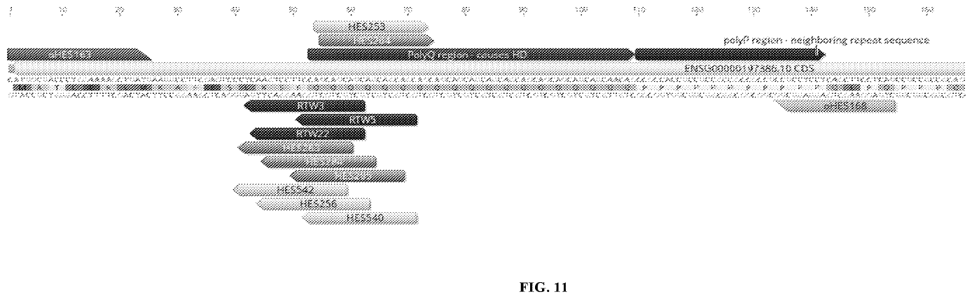

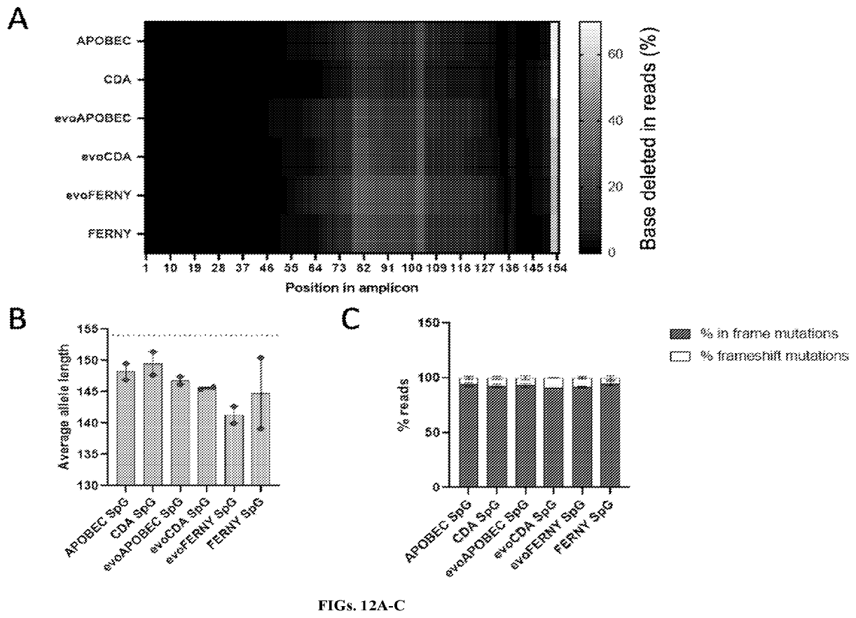

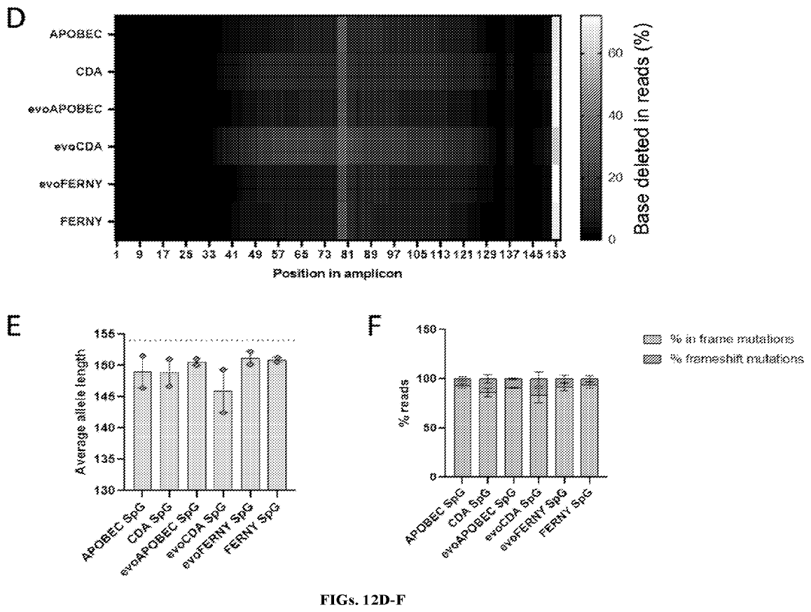

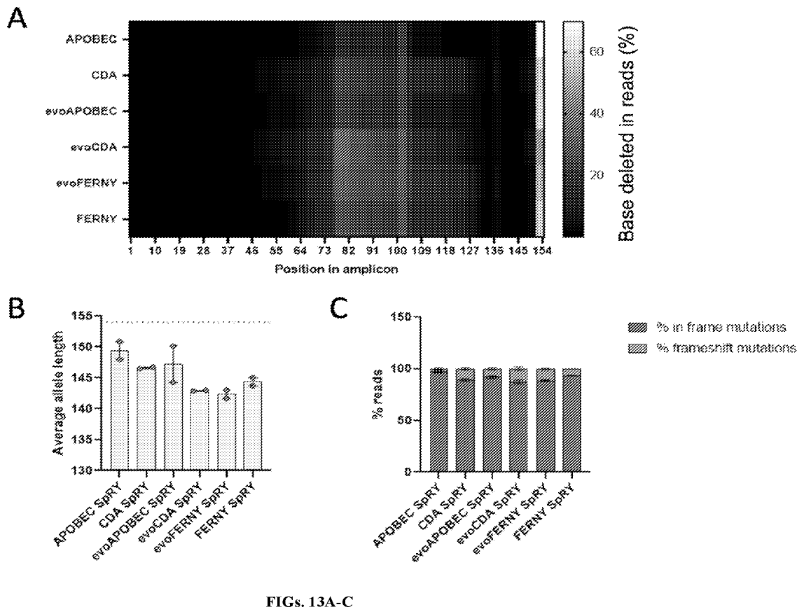

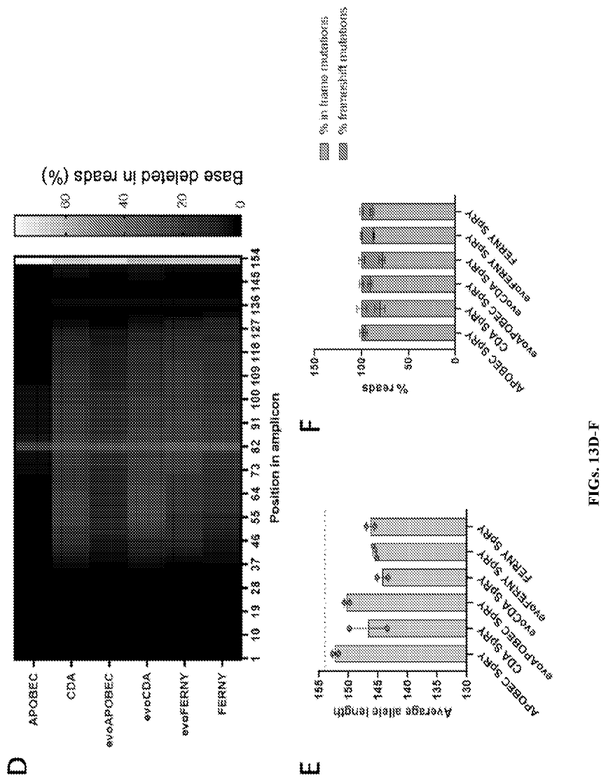

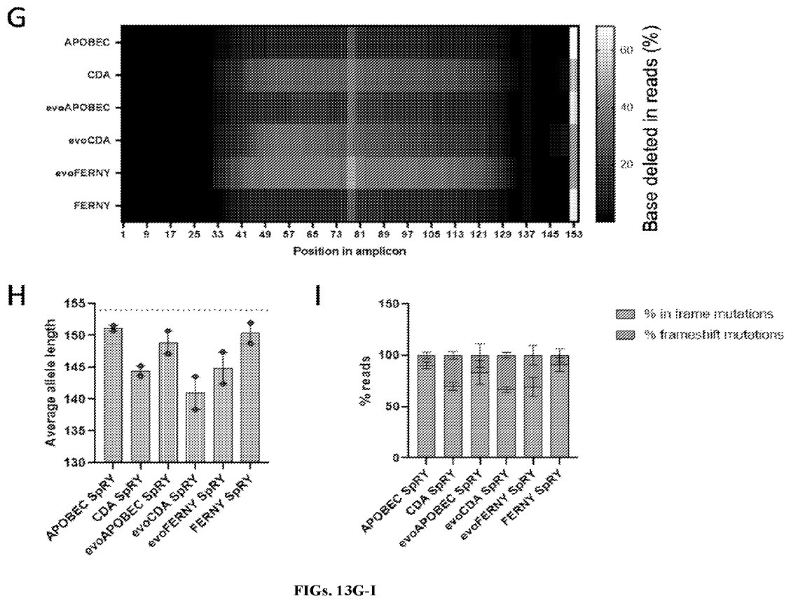

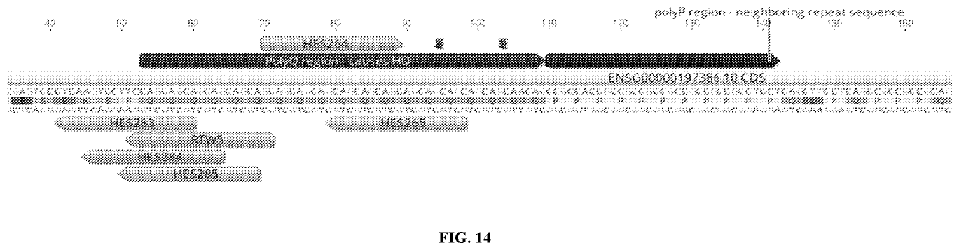

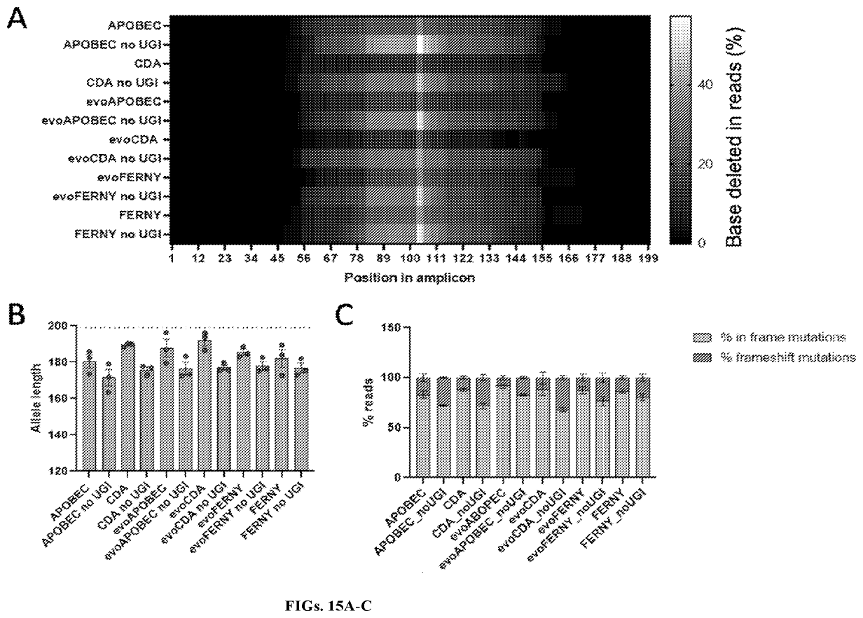

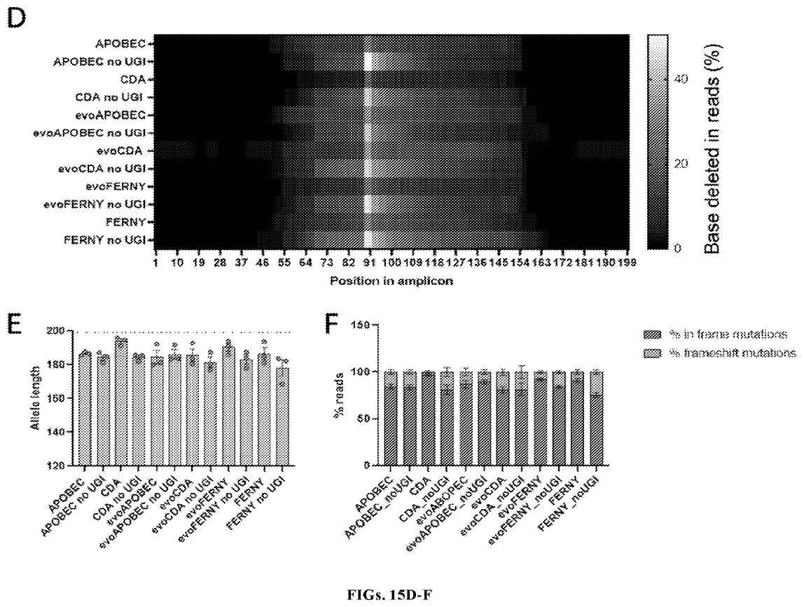

To determine whether deamination events can trigger BER within the CAG-repeat and lead to repeat contraction, we initially tested various BEs with gRNAs (FIG. 11 and Table 1) that positioned the editing windows within the CAG-repeat. We assessed the efficiencies of either SpG-BEs or SpRY-BEs (FIGS. 12 and 13, respectively) bearing the conventional rAPOBEC1 deaminase (BE4max), or alternate deaminases cloned into the BE4max CBE architecture including CDA1^{20,60,66}. Additionally, we explored engineered deaminase domains that have been reported to more efficiently edit cytosines located within a GC sequence context (including evoAPOBEC1, evoCDA, evoFERNY and FERNY)⁶¹ would improve editing in the CAGCAG and CTGCTG repeats encoded on the coding and non-coding strands, respectively. We co-transfected combinations of plasmids expressing the enzymes and gRNAs into HEK293T cells, extracted genomic DNA at 72 hours post-transfection, and the ability of each BE construct to contract the repeat was be assessed by NGS.

We performed experiments with SpG-based CBEs and two gRNAs whose edit windows were either positioned on the top strand fully within the repeat (FIGS. 12A-12C) or positioned on the bottom strand and at the 5′ border of the CAG repeat (FIGS. 12D-12F). We observed editing deletion of some bases in the CAG repeat up to approximately 30% (FIGS. 12A and 12D, with deletion of 3 or 4 CAG repeats (FIGS. 12B and 12E), and most alleles remaining in-frame (FIGS. 12C and 12F). We then assessed the efficiency of editing when using SpRY-based CBEs and three different gRNAs either targeted to the top strand embedded within the repeat (FIGS. 13A-C), positioned on the bottom strand at the 5′ border of the CAG repeat (FIGS. 13D-13F), or on the top strand at the 3′ border of the CAG repeat/spanning the poly-P region, positioning the deamination event in the poly-P region (FIGS. 13G-131). The levels of editing and deletion were dependent on the gRNA target site (and likely the position and efficiency of the base edit), where in some cases we observed nearly 50% editing and deletion of up to 5 CAG repeats (FIGS. 13H-I).

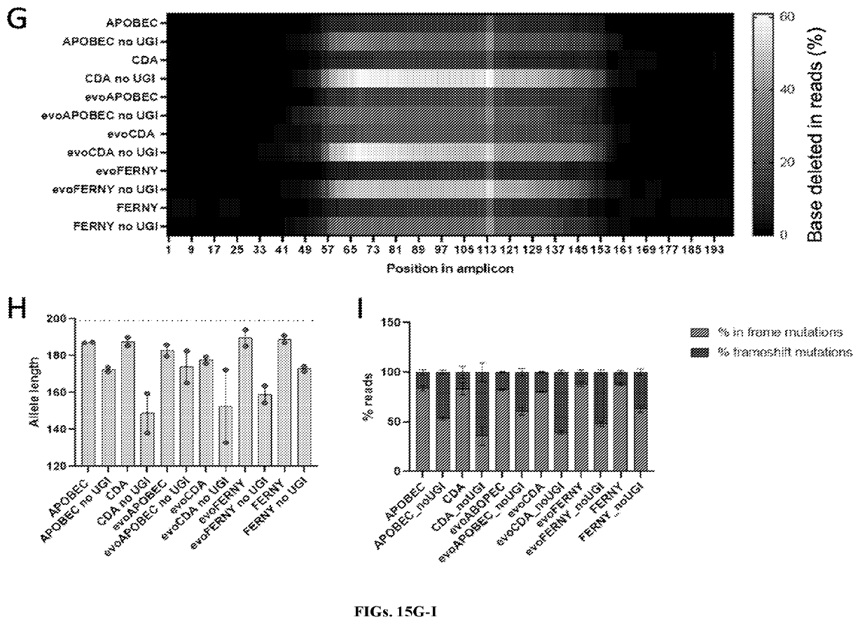

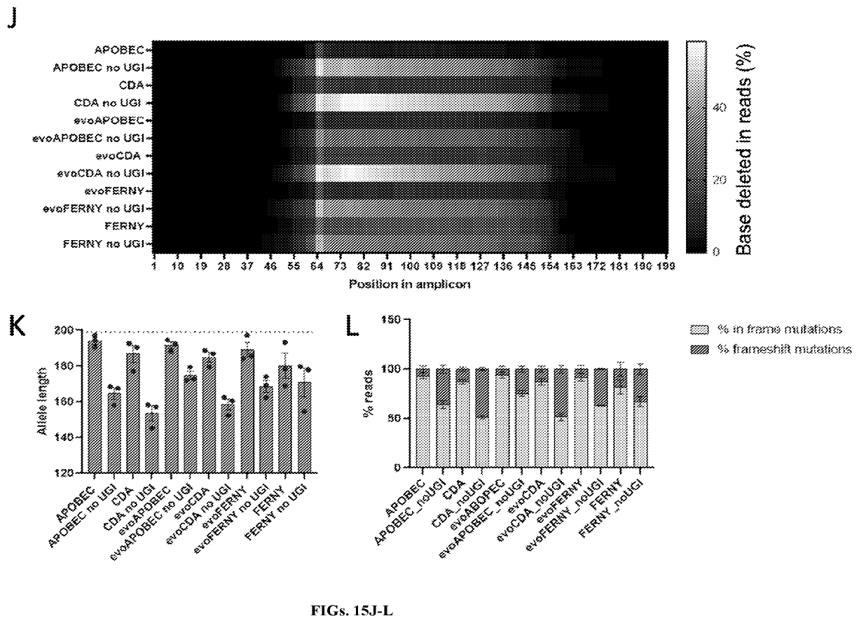

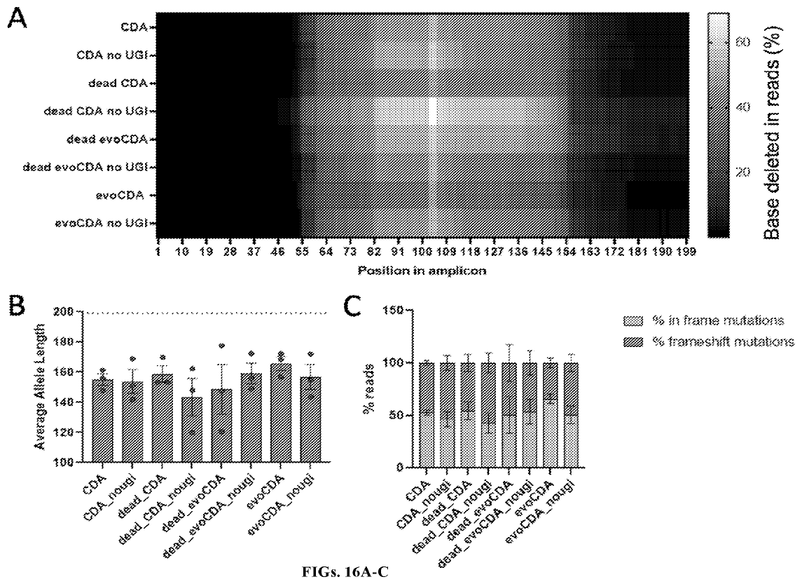

Next, we assessed additional editors using somewhat different set of SpCas9 gRNAs that would position the edit window of the base editors or repeat editors within different phases of the CAG repeat (FIG. 14 and Table 1). We tested a further engineered set of SpG-derived CBEs (containing 2×UGI domains) and REds (without the UGI domains) harboring various deaminase domains (rAPOBEC1, CDA1, evoAPOBEC, evoCDA, evoFERNY, and FERNY) with four different gRNAs. The gRNA target sites were either fully embedded within the CAG repeat targeted to the top strand (FIGS. 15A-15C), fully embedded within the CAG repeat targeted to the bottom strand (FIGS. 15D-15F), positioned on the bottom strand at the 5′ border of the CAG repeat where the edit window is further into the repeat (FIGS. 15G-15I), and positioned on the bottom strand at the 5′ border of the CAG repeat where the edit window is overlaps the exon-repeat junction (FIGS. 15J-15L). Depending on the positioning of the gRNA and BE or REd used, we observed a range of different edit efficiencies and deletion/repeat contraction sizes. Edits shorter in length predominantly led to in-frame contractions (FIGS. 15A-15F), compared to certain longer edits that tended to occur more often out-of-frame (FIGS. 15G-15L). In general, the UGI-less REd constructs led to more efficient editing and contraction (leading to over 50% deletion in some alleles and ablation of more than 15 CAG repeats; FIGS. 15G and 15H, respectively), which is consistent with our hypothesis/model that uracil glycosylase creating an abasic site could initiate BER and cause longer contractions/deletions (FIG. 5A).

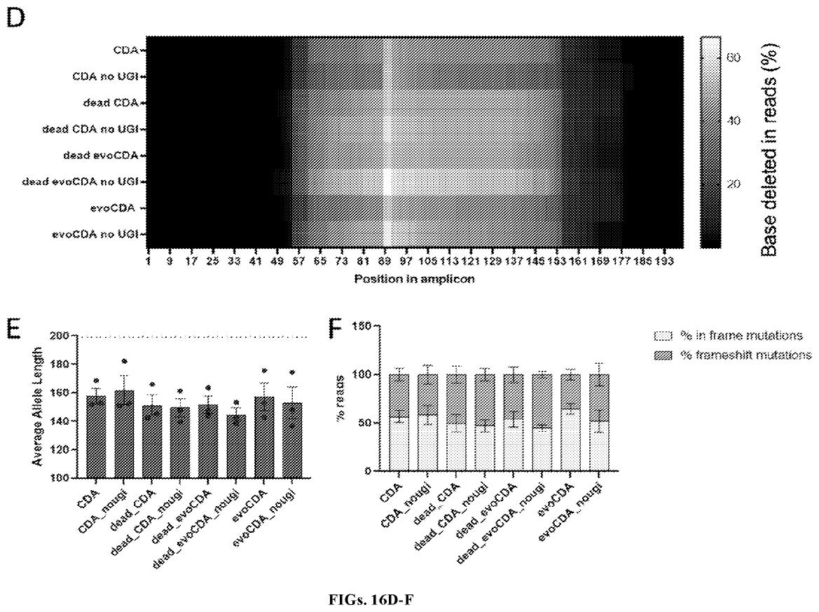

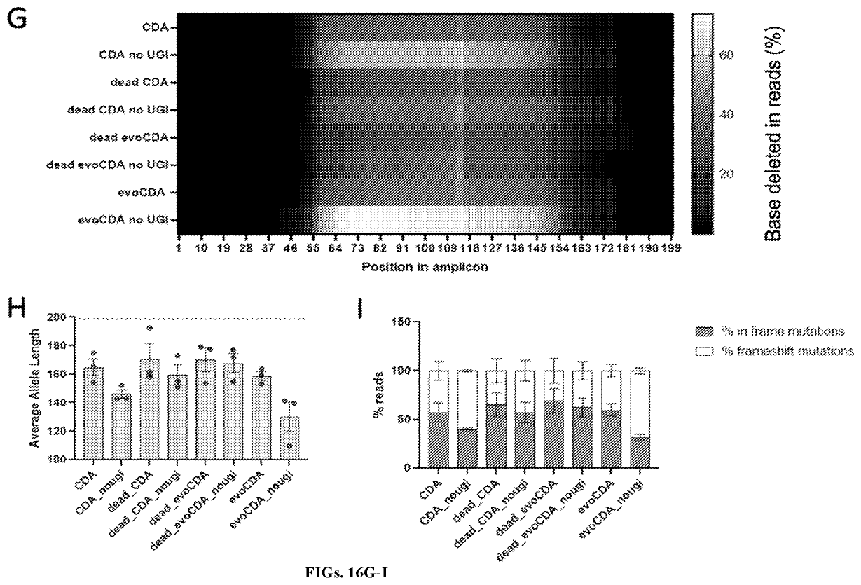

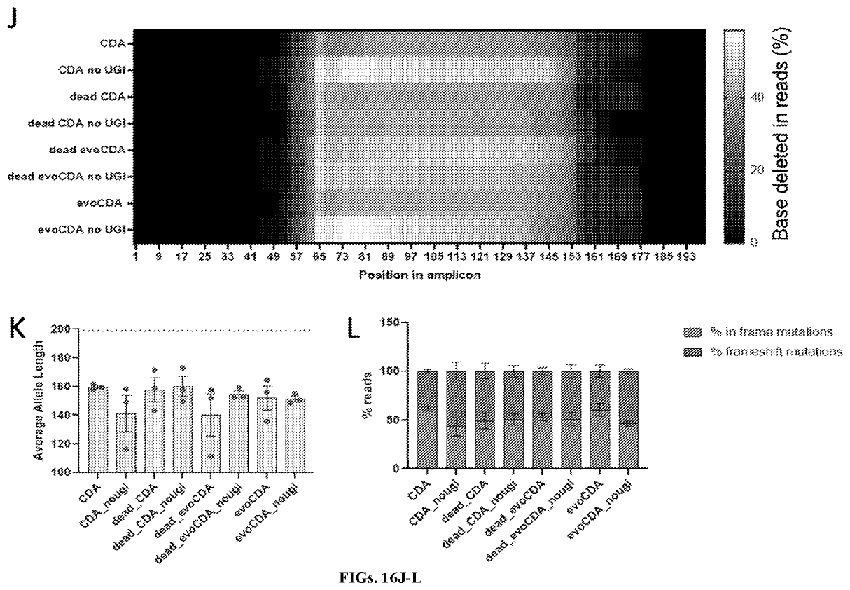

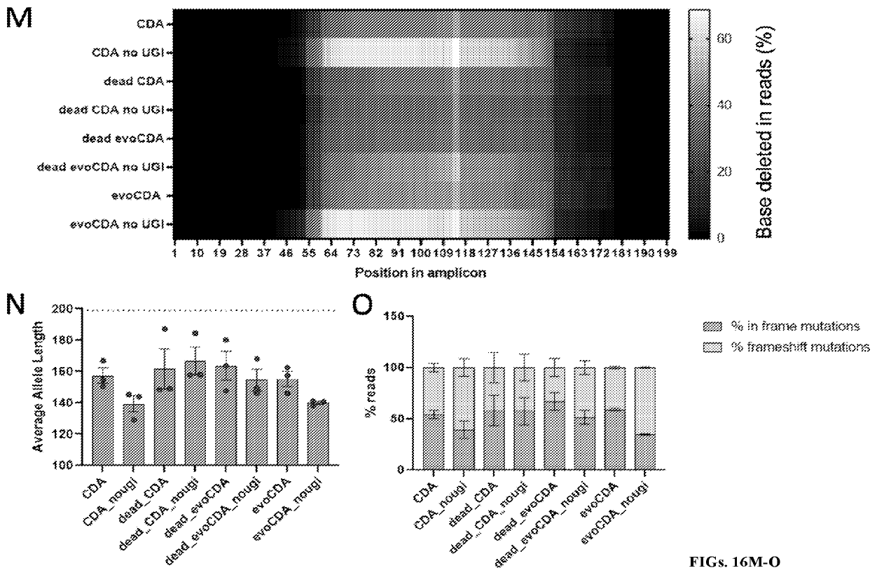

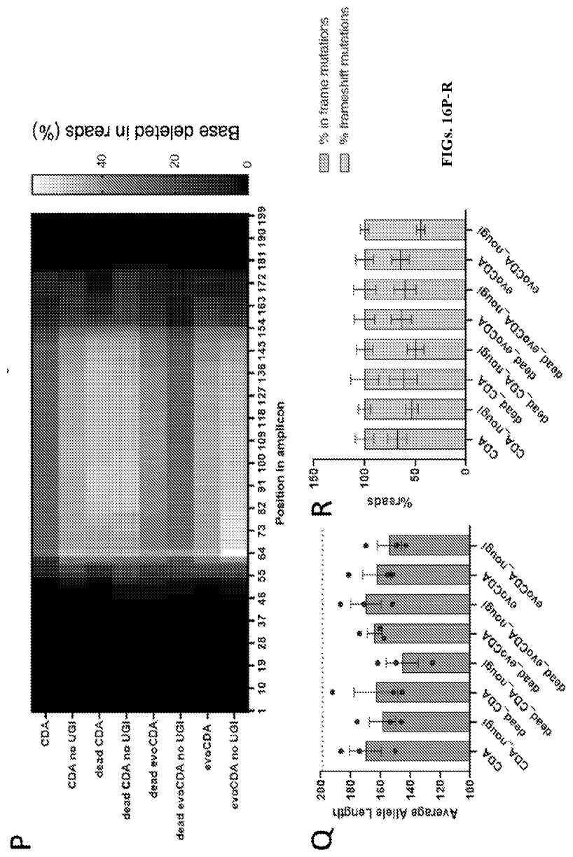

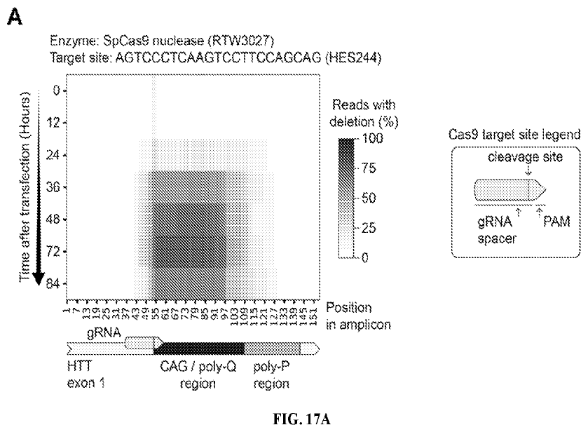

We then examined editing efficiencies when testing SpG versions of CBEs or REds with without the UGI, respectively, and either as nickases (nCas9, D10A causing RuvC nuclease inactivation) or as catalytically dead Cas9 enzymes (dCas9, additionally encoding the H840A mutation to inactivate the HNH domain). We tested a total of 8 different editors bearing these four configurations and harboring either the CDA1 deaminase or evoCDA. These eight constructs were assessed for editing and contraction of the HTT exon 1 CAG repeat using six different gRNAs (FIG. 14 and Table 1); the four gRNAs used above (as described in FIG. 15A-15L, now used in FIGS. 16A-16L), and two additional gRNAs targeting the bottom strand that position the edit window near the junction of exon 1 and the 5′ end of the CAG repeat (all six gRNAs shown in FIG. 14). We observed several combinations of editors and gRNAs that approached or exceeded 60% deletion (FIGS. 16A, 16D, 16G, 16J, 16M, and 16P), with some alleles harboring a complete deletion of the CAG repeat (and sometimes even into the flanking exon 1 or poly-P sequence). Depending on the gRNA used, the dCas9 versions of the CBEs or REds led to higher efficiency deletions than the nCas9 versions (though some led to lower editing).

Together, these results reveal that engineered base editors and repeat editors (using alternate deaminase domains, optionally without the UGI domains, and either as nCas9 or dCas9 enzymes) were proficient at creating deletions in the HTT CAG repeat. High levels of editing across the length of the CAG repeat were achieved, with many cases leading to >50% of edited alleles remaining in-frame. The positioning of the gRNA target site played a role in defining the edit profile, by controlling the position of the deamination event and the optional DNA nick. Our results establish a set of technologies capable of efficient repeat contraction.

### Example 1.6: Timecourse Experiments to Analyze CAG Contraction

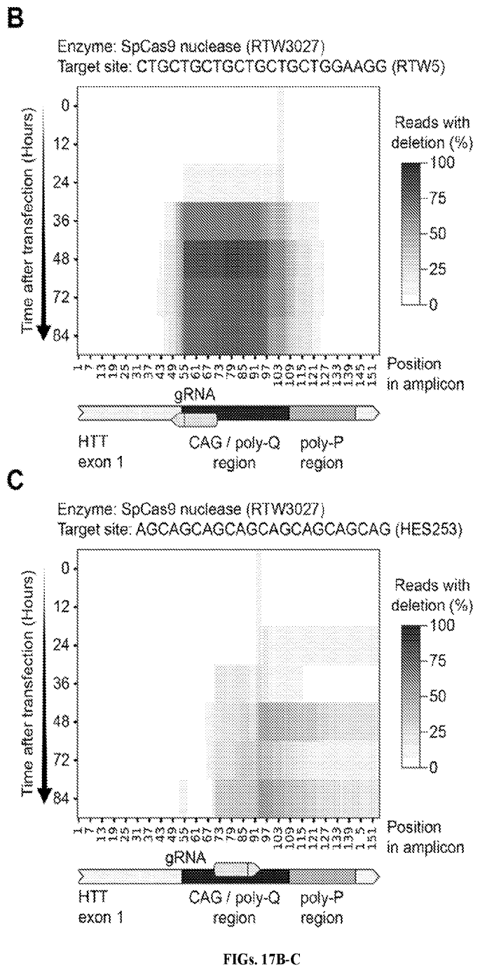

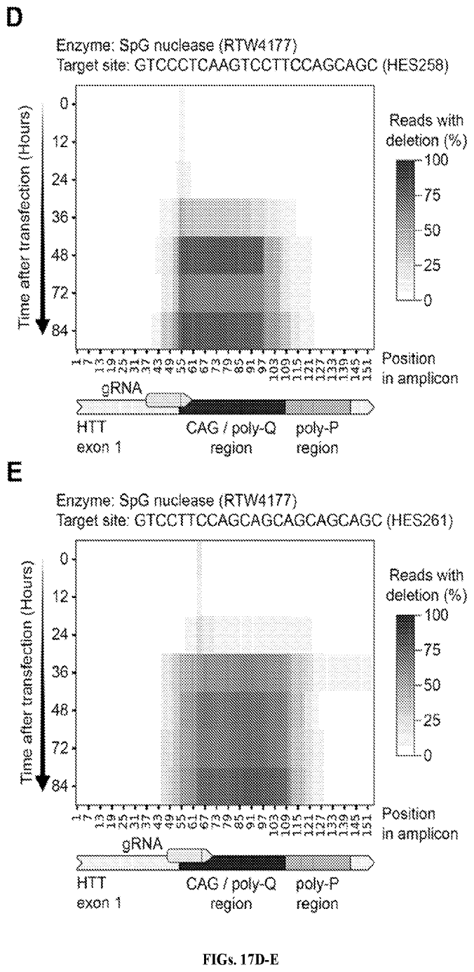

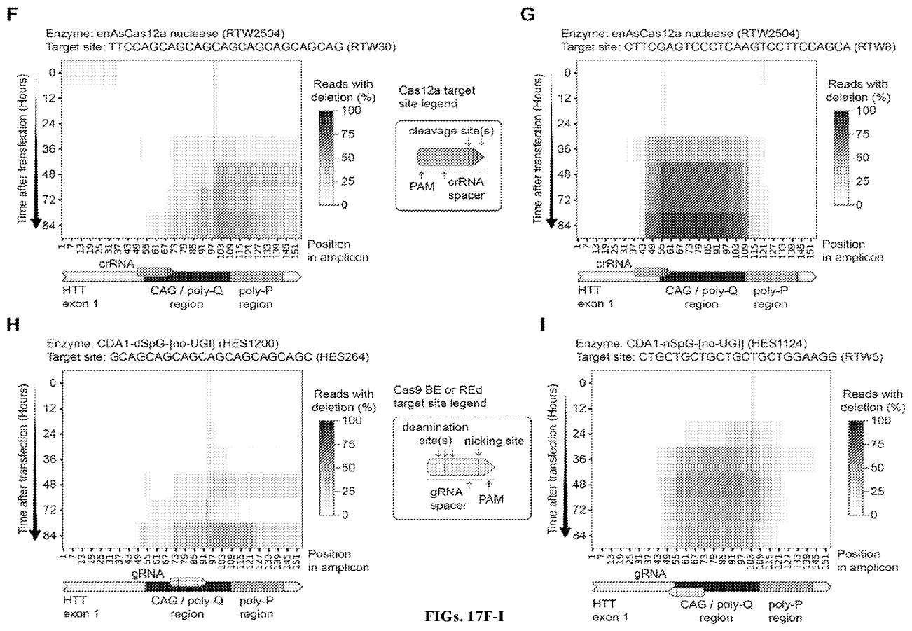

Next we performed experiments to assess the ability of the various Cas9 nuclease, Cas12a nuclease, base editor, and repeat editors constructs, combined with various gRNAs, to contract the CAG-repeat over time. To do so, we transfected enzyme and gRNA expression plasmids into human HEK293T cells. Editing efficiency and repeat contraction was assessed by extracting genomic DNA at various time-points between 12 and 84 hours post-transfection, and performing PCR to amplify the HTT exon-1 region (FIG. 3b). The extent of CAG-repeat reduction was determined by NGS and visualized in heatmaps (FIGS. 17A-171). Depending on the construct and gRNA, we generally observed the initial appearance of deletions within the repeat around 24-48 hours post transfection. There was also variability in the kinetics of deletion over time amongst the editors and gRNAs tested, with some conditions reaching saturated levels of deletion early (by ~48 hours) and some continuing to increase in editing at the final timepoint (84 hours). Certain Cas9 (SpCas9 and SpG) or Cas12a nuclease and gRNA conditions exhibited more rapid and efficient repeat contraction (FIGS. 17A, 17B, 17D, 17E, and 17G), which appeared to occur when the cleavage site of the gRNA/enzyme was situated more proximally to the 5′ end of the CAG repeat. Targeting across the exon ⅕′ repeat boundary typically led to deletions confined within the CAG repeat, whereas target sites mostly or fully embedded within the repeat often caused deletions that extended outside of the CAG repeat. The DNA strand being targeted by the enzyme/gRNA did not appear to play a large role in the deletion pattern, although this (and other) observations merit further investigation. These results provide additional insight into the kinetics of repeat contraction in human cells and suggest that certain enzyme/gRNA combinations are more effective than others at precisely deleting the CAG repeat.

### REFERENCES

1. Paulson, H. Repeat expansion diseases. in Handbook of Clinical Neurology (eds. Geschwind, D. H., Paulson, H. L. & Klein, C.) vol. 147 105-123 (Elsevier, 2018).
2. Gatchel, J. R. & Zoghbi, H. Y. Diseases of Unstable Repeat Expansion: Mechanisms and Common Principles. Nat. Rev. Genet. 6, 743-755 (2005).
3. Polleys, E. J., House, N. C. M. & Freudenreich, C. H. Role of recombination and replication fork restart in repeat instability. DNA Repair 56, 156-165 (2017).
4. Richard, G.-F. Shortening trinucleotide repeats using highly specific endonucleases: a possible approach to gene therapy? Trends Genet. 31, 177-186 (2015).
5. Orr, H. T. & Zoghbi, H. Y. Trinucleotide Repeat Disorders. Annu. Rev. Neurosci. 30, 575-621 (2007).
6. The Huntington's Disease Collaborative Research Group. A novel gene containing a trinucleotide repeat that is expanded and unstable on Huntington's disease chromosomes. Cell 72, 971-983 (1993).
7. Budworth, H. & McMurray, C. T. A Brief History of Triplet Repeat Diseases. in Trinucleotide Repeat Protocols (eds. Kohwi, Y. & McMurray, C. T.) 3-17 (Humana Press, 2013). doi: 10.1007/978-1-62703-411-1_1.
8. Labbadia, J. & Morimoto, R. I. Huntington's disease: underlying molecular mechanisms and emerging concepts. Trends Biochem. Sci. 38, 378-385 (2013).
9. McColgan, P. & Tabrizi, S. J. Huntington's disease: a clinical review. Eur. J. Neurol. 25, 24-34 (2018).
10. Gaj, T., Gersbach, C. A. & Barbas, C. F. ZFN, TALEN, and CRISPR/Cas-based methods for genome engineering. Trends Biotechnol. 31, 397-405 (2013).
11. Doudna, J. A. & Charpentier, E. The new frontier of genome engineering with CRISPR-Cas9. Science 346, (2014).
12. Sander, J. D. & Joung, J. K. CRISPR-Cas systems for editing, regulating and targeting genomes. Nat. Biotechnol. 32, 347-355 (2014).
13. Hsu, P. D., Lander, E. S. & Zhang, F. Development and applications of CRISPR-Cas9 for genome engineering. Cell 157, 1262-1278 (2014).
14. Maeder, M. L. & Gersbach, C. A. Genome-editing Technologies for Gene and Cell Therapy. Mol. Ther. 24, 430-446 (2016).
15. Cubbon, A., Ivancic-Bace, I. & Bolt, E. L. CRISPR-Cas immunity, DNA repair and genome stability. Biosci. Rep. 38, (2018).
16. Anzalone, A. V., Koblan, L. W. & Liu, D. R. Genome editing with CRISPR-Cas nucleases, base editors, transposases and prime editors. Nat. Biotechnol. 38, 824-844 (2020).
17. Adli, M. The CRISPR tool kit for genome editing and beyond. Nat. Commun. 9, 1911 (2018).
18. Thakore, P. I., Black, J. B., Hilton, I. B. & Gersbach, C. A. Editing the epigenome: technologies for programmable transcription and epigenetic modulation. Nat. Methods 13, 127-137 (2016).
19. Dominguez, A. A., Lim, W. A. & Qi, L. S. Beyond editing: repurposing CRISPR-Cas9 for precision genome regulation and interrogation. Nat. Rev. Mol. Cell Biol. 17, 5-15 (2016).
20. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
21. Komor, A. C., Badran, A. H. & Liu, D. R. Editing the Genome Without Double-Stranded DNA Breaks. ACS Chem. Biol. 13, 383-388 (2018).
22. Rees, H. A. & Liu, D. R. Base editing: precision chemistry on the genome and transcriptome of living cells. Nat. Rev. Genet. 19, 770-788 (2018).
23. Anzalone, A. V. et al. Search-and-replace genome editing without double-strand breaks or donor DNA. Nature 576, 149-157 (2019).
24. Jinek, M. et al. A Programmable Dual-RNA-Guided DNA Endonuclease in Adaptive Bacterial Immunity. Science 337, 816-821 (2012).
25. Gasiunas, G., Barrangou, R., Horvath, P. & Siksnys, V. Cas9-crRNA ribonucleoprotein complex mediates specific DNA cleavage for adaptive immunity in bacteria. Proc. Natl. Acad. Sci. 109, E2579-E2586 (2012).
26. Cong, L. et al. Multiplex Genome Engineering Using CRISPR/Cas Systems. Science 339, 819-823 (2013).
27. Tsai, S. Q. & Joung, J. K. Defining and improving the genome-wide specificities of CRISPR-Cas9 nucleases. Nat. Rev. Genet. 17, 300-312 (2016).
28. Shin, J. W. et al. Permanent inactivation of Huntington's disease mutation by personalized allele-specific CRISPR/Cas9. Hum. Mol. Genet. 25, 4566-4576 (2016).
29. Monteys, A. M., Ebanks, S. A., Keiser, M. S. & Davidson, B. L. CRISPR/Cas9 Editing of the Mutant Huntingtin Allele In Vitro and In Vivo. Mol. Ther. 25, 12-23 (2017).
30. Cinesi, C., Aeschbach, L., Yang, B. & Dion, V. Contracting CAG/CTG repeats using the CRISPR-Cas9 nickase. Nat. Commun. 7, 13272 (2016).
31. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
32 Kleinstiver, B. P. et al. Engineered CRISPR-Cas9 nucleases with altered PAM specificities. Nature 523, 481-485 (2015).
33. Kleinstiver, B. P. et al. Broadening the targeting range of Staphylococcus aureus CRISPR-Cas9 by modifying PAM recognition. Nat. Biotechnol. 33, 1293-1298 (2015).
34. Ran, F. A. et al. In vivo genome editing using Staphylococcus aureus Cas9. Nature 520, 186-191 (2015).
35. Kleinstiver, B. P. et al. Genome-wide specificities of CRISPR-Cas Cpf1 nucleases in human cells. Nat. Biotechnol. 34, 869-874 (2016).
36. Zetsche, B. et al. Cpf1 Is a Single RNA-Guided Endonuclease of a Class 2 CRISPR-Cas System. Cell 163, 759-771 (2015).
37. Kim, D. et al. Genome-wide analysis reveals specificities of Cpf1 endonucleases in human cells. Nat. Biotechnol. 34, 863-868 (2016).
38. Fonfara, I., Richter, H., Bratovič, M., Le Rhun, A. & Charpentier, E. The CRISPR-associated DNA-cleaving enzyme Cpf1 also processes precursor CRISPR RNA. Nature 532, 517-521 (2016).
39. Zetsche, B. et al. Multiplex gene editing by CRISPR-Cpf1 using a single crRNA array. Nat. Biotechnol. 35, 31-34 (2017).
40. Tak, Y. E. et al. Inducible and multiplex gene regulation using CRISPR-Cpf1-based transcription factors. Nat. Methods 14, 1163-1166 (2017).
41. Kleinstiver, B. P. et al. Engineered CRISPR-Cas12a variants with increased activities and improved targeting ranges for gene, epigenetic and base editing. Nat. Biotechnol. 37, 276-282 (2019).
42. van Overbeek, M. et al. DNA Repair Profiling Reveals Nonrandom Outcomes at Cas9-Mediated Breaks. Mol. Cell 63, 633-646 (2016).
43. White, J. K. et al. Huntingtin is required for neurogenesis and is not impaired by the Huntington's disease CAG expansion. Nat. Genet. 17, 404-410 (1997).
44. Trettel, F. et al. Dominant phenotypes produced by the HD mutation in STHdhQ111 striatal cells. Hum. Mol. Genet. 9, 2799-2809 (2000).
45. De Mello, W. C., Gerena, Y. & Ayala-Peña, S. Angiotensins and Huntington's Disease: A Study on Immortalized Progenitor Striatal Cell Lines. Front. Endocrinol. 8, (2017).
46. Bae, S., Kweon, J., Kim, H. S. & Kim, J.-S. Microhomology-based choice of Cas9 nuclease target sites. Nat. Methods 11, 705-706 (2014).
47. Shen, M. W. et al. Predictable and precise template-free CRISPR editing of pathogenic variants. Nature 563, 646-651 (2018).
48. Kim, H. K. et al. SpCas9 activity prediction by DeepSpCas9, a deep learning-based model with high generalization performance. Sci. Adv. 5, caax9249 (2019).
49. Mali, P. et al. CAS9 transcriptional activators for target specificity screening and paired nickases for cooperative genome engineering. Nat. Biotechnol. 31, 833-838 (2013).
50. Ran, F. A. et al. Double Nicking by RNA-Guided CRISPR Cas9 for Enhanced Genome Editing Specificity. Cell 154, 1380-1389 (2013).
51. Clarke, R. et al. Enhanced Bacterial Immunity and Mammalian Genome Editing via RNA-Polymerase-Mediated Dislodging of Cas9 from Double-Strand DNA Breaks. Mol. Cell 71, 42-55.e8 (2018).
52. Clement, K. et al. CRISPResso2 provides accurate and rapid genome editing sequence analysis. Nat. Biotechnol. 37, 224-226 (2019).
53. Usdin, K., House, N. C. M. & Freudenreich, C. H. Repeat instability during DNA repair: Insights from model systems. Crit. Rev. Biochem. Mol. Biol. 50, 142-167 (2015).
54. Sundararajan, R., Gellon, L., Zunder, R. M. & Freudenreich, C. H. Double-Strand Break Repair Pathways Protect against CAG/CTG Repeat Expansions, Contractions and Repeat-Mediated Chromosomal Fragility in Saccharomyces cerevisiae. Genetics 184, 65-77 (2010).
55. Liu, Y. & Wilson, S. H. DNA base excision repair: a mechanism of trinucleotide repeat expansion. Trends Biochem. Sci. 37, 162-172 (2012).
56. Beaver, J. M. et al. AP endonuclease 1 prevents trinucleotide repeat expansion via a novel mechanism during base excision repair. Nucleic Acids Res. 43, 5948-5960 (2015).
57. Jones, L., Houlden, H. & Tabrizi, S. J. DNA repair in the trinucleotide repeat disorders. Lancet Neurol. 16, 88-96 (2017).
58. Su, X. A. & Freudenreich, C. H. Cytosine deamination and base excision repair cause R-loop-induced CAG repeat fragility and instability in Saccharomyces cerevisiae. Proc. Natl. Acad. Sci. 114, E8392-E8401 (2017).
59. Freudenreich, C. H. R-loops: targets for nuclease cleavage and repeat instability. Curr. Genet. 64, 789-794 (2018).
60. Nishida, K. et al. Targeted nucleotide editing using hybrid prokaryotic and vertebrate adaptive immune systems. Science 353, (2016).
61. Thuronyi, B. W. et al. Continuous evolution of base editors with expanded target compatibility and improved activity. Nat. Biotechnol. 37, 1070-1079 (2019).
62. Gaudelli, N. M. et al. Programmable base editing of A·T to G·C in genomic DNA without DNA cleavage. Nature 551, 464-471 (2017).
63. Richter, M. F. et al. Phage-assisted evolution of an adenine base editor with improved Cas domain compatibility and activity. Nat. Biotechnol. 38, 883-891 (2020).
64. Gaudelli, N. M. et al. Directed evolution of adenine base editors with increased activity and therapeutic application. Nat. Biotechnol. 38, 892-900 (2020).
65. Huang, T. P., Newby, G. A. & Liu, D. R. Precision genome editing using cytosine and adenine base editors in mammalian cells. Nat. Protoc. 16, 1089-1128 (2021).
66. Koblan, L. W. et al. Improving cytidine and adenine base editors by expression optimization and ancestral reconstruction. Nat. Biotechnol. 36, 843-846 (2018).
67. Li, X. et al. Base editing with a Cpf1-cytidine deaminase fusion. Nat. Biotechnol. 36, 324-327 (2018).
68. Gibson, D. G. et al. Enzymatic assembly of DNA molecules up to several hundred kilobases. Nat. Methods 6, 343-345 (2009).
69. Rohland, N. & Reich, D. Cost-effective, high-throughput DNA sequencing libraries for multiplexed target capture. Genome Res. 22, 939-946 (2012).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of contracting a nucleotide repeat expansion in a cell, the method comprising contacting the cell with or expressing in the cell a Cas-based enzyme and a guide RNA (gRNA) that directs the Cas-based enzyme to the nucleotide repeat, preferably wherein the gRNA binds to an exon/repeat border, in an amount sufficient to reduce the number of nucleotide repeats in the cell.

2. A method of treating a subject who has a condition associated with nucleotide repeat expansion, optionally CAG nucleotide repeat expansion in a HTT gene, the method comprising administering to the subject a therapeutically effective amount of a Cas-based enzyme and a guide RNA that directs the Cas-based enzyme to the nucleotide repeat, preferably wherein the gRNA binds to an exon/repeat border, in an amount sufficient to reduce the number of nucleotide repeats in the cell.

3. The method of claim 2, wherein the Cas-based enzyme and gRNA are administered to the CNS, optionally brain or spinal cord of the subject (optionally via ICV, cisternae magna, or intrathecal administration), or administered systemically to the subject.

4. The method of claim 1, wherein the Cas-based enzyme comprises Cas9, optionally SpG, or SpRY, or Cas12a, optionally enAsCas12a.

5. The method of claim 4, wherein the Cas-based enzyme is:
(i) a Cas9 nickase, optionally a SpG nickase, or SpRY nickase;
(ii) a Cas12a nickase, optionally a enAsCas 12a nickase;
(iii) a Cas9- or Cas12a-Base editor (BE) comprising a nicking or catalytically inactive Cas9, SpG, SpRY, Cas12a, or enAsCas12a and a UGI, or
(iv) a Cas9- or Cas12a-Repeat editor comprising a nicking or catalytically inactive Cas9, SpG, SpRY, Cas12a, or enAsCas12a and lacking a UGI.

6. The method of claim 1, wherein the gRNA is listed in Table 1 or 2.

7. The method of claim 6, wherein the Cas-based enzyme and gRNA are as listed in Table 4.

8. The method of claim 2, wherein the Cas-based enzyme and gRNA are administered in an expression vector, optionally a plasmid or viral vector; are administered as mRNA; or are administered as RNPs.

9. The method of claim 2, wherein the number of CAG repeats is reduced to below 40 or below 26.

10. A composition comprising a Cas-based enzyme and gRNA as described herein, optionally as listed in Table 4.

11. A nucleic acid encoding a Cas-based enzyme and gRNA as described herein, optionally as listed in Table 4.


SEQUENCE LISTING (178 sequences)

SEQ ID NO: 1 - protein, 1368 aa, Streptococcus pyogenes [sequence omitted]

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - DNA, 20 bp, synthetic construct
  source 1..20: mol_type = other DNA
gcagcagcagcagcagcagc

SEQ ID NO: 4 - DNA, 21 bp, synthetic construct
  source 1..21: mol_type = other DNA
gtgctgctggaaggacttgag

SEQ ID NO: 5 - DNA, 21 bp, synthetic construct
  source 1..21: mol_type = other DNA
gagcagcagcagcagcagcag

SEQ ID NO: 6 - DNA, 21 bp, synthetic construct
  source 1..21: mol_type = other DNA
gtgctgctgctggaaggactt

SEQ ID NO: 7 - DNA, 21 bp, synthetic construct
  source 1..21: mol_type = other DNA
gcgccgccgccgccgcctcct

SEQ ID NO: 8 - DNA, 20 bp, synthetic construct
  source 1..20: mol_type = other DNA
gctgctgctgctgctgctgc

SEQ ID NO: 9 - DNA, 21 bp, synthetic construct
  source 1..21: mol_type = other DNA
gctgctgctgctggaaggact

SEQ ID NO: 10 - DNA, 21 bp, synthetic construct
  source 1..21: mol_type = other DNA
gctgctgctgctgctgctgga

SEQ ID NO: 11 - DNA, 21 bp, synthetic construct
  source 1..21: mol_type = other DNA
gtgctgctgctgctgctggaa

SEQ ID NO: 12 - protein, 495 aa, unidentified
MAVVYVISKSGKPLMPTTRCGHVRILLKEGKARVVERKPFTIQLTYESAEETQPLVLGIDPGRTNIGMSVVTESGESVFNAQIETRNKDVPKLMKDRKQYRMAHRRLKRRCKRRRRAKAAGTAFEEGEKQRLLPGCFKPITCKSIRNKEARFNNRKRPVGWLTPTANHLLVTHLNVVKKVQKILPVAKVVLELNRFSFMAMNNPKVQRWQYQRGPLYGKGSVEEAVSMQQDGHCLFCKHGIDHYHHVVPRRKNGSETLENRVGLCEEHHRLVHTDKEWEANLASKKSGMNKKYHALSVLNQIIPYLADQLADMFPGNFCVTSGQDTYLFREEHGIPKDHYLDAYCIACSALTDAKKVSSPKGRPYMVHQFRRHDRQACHKANLNRSYYMGGKLVATNRHKAMDQKTDSLEEYRAAHSAADVSKLTVKHPSAQYKDMSRIMPGSILVSGEGKLFTLSRSEGRNKGQVNYFVSTEGIKYWARKCQYLRNNGGLQIYV

SEQ ID NO: 13 - DNA, 23 bp, synthetic construct
  source 1..23: mol_type = other DNA
ctgctgctggaaggacttgaggg

SEQ ID NO: 14 - DNA, 21 bp, synthetic construct
  source 1..21: mol_type = other DNA
gctgctgctggaaggacttga

SEQ ID NO: 15 - DNA, 23 bp, synthetic construct
  source 1..23: mol_type = other DNA
gctgctgctggaaggacttgagg

SEQ ID NO: 16 - DNA, 20 bp, synthetic construct
  source 1..20: mol_type = other DNA
gctgctgctggaaggacttg

SEQ ID NO: 17 - DNA, 23 bp, synthetic construct
  source 1..23: mol_type = other DNA
ctgctgctgctgctgctggaagg

SEQ ID NO: 18 - DNA, 23 bp, synthetic construct
  source 1..23: mol_type = other DNA
gctgctgctgctgctgctgctgg

SEQ ID NO: 19 - DNA, 23 bp, synthetic construct
  source 1..23: mol_type = other DNA
gaaggacttgagggactcgaagg

SEQ ID NO: 20 - DNA, 20 bp, synthetic construct
  source 1..20: mol_type = other DNA
gaaggacttgagggactcga

SEQ ID NO: 21 - DNA, 23 bp, synthetic construct
  source 1..23: mol_type = other DNA
ggcggcggcggcggcggtggcgg

SEQ ID NO: 22 - DNA, 20 bp, synthetic construct
  source 1..20: mol_type = other DNA
ggcggcggcggcggcggtgg

SEQ ID NO: 23 - DNA, 23 bp, synthetic construct
  source 1..23: mol_type = other DNA
ggcggcggcggcggcggcggtgg

SEQ ID NO: 24 - DNA, 20 bp, synthetic construct
  source 1..20: mol_type = other DNA
ggcggcggcggcggcggcgg

SEQ ID NO: 25 - DNA, 23 bp, synthetic construct
  source 1..23: mol_type = other DNA
ggaagctgaggaggcggcggcgg

SEQ ID NO: 26 - DNA, 20 bp, synthetic construct
  source 1..20: mol_type = other DNA
ggaagctgaggaggcggcgg

SEQ ID NO: 27 - DNA, 23 bp, synthetic construct
  source 1..23: mol_type = other DNA
tgaggaagctgaggaggcggcgg

SEQ ID NO: 28 - DNA, 21 bp, synthetic construct
  source 1..21: mol_type = other DNA
gtgaggaagctgaggaggcgg

SEQ ID NO: 29 - DNA, 23 bp, synthetic construct
  source 1..23: mol_type = other DNA
ggctgaggaagctgaggaggcgg

SEQ ID NO: 30 - DNA, 20 bp, synthetic construct
  source 1..20: mol_type = other DNA
ggctgaggaagctgaggagg

SEQ ID NO: 31 - DNA, 23 bp, synthetic construct
  source 1..23: mol_type = other DNA
ggcggctgaggaagctgaggagg

SEQ ID NO: 32 - DNA, 20 bp, synthetic construct
  source 1..20: mol_type = other DNA
ggcggctgaggaagctgagg

SEQ ID NO: 33 - DNA, 23 bp, synthetic construct
  source 1..23: mol_type = other DNA
ggcggcggctgaggaagctgagg

SEQ ID NO: 34 - DNA, 20 bp, synthetic construct
  source 1..20: mol_type = other DNA
ggcggcggctgaggaagctg

SEQ ID NO: 35 - DNA, 23 bp, synthetic construct
  source 1..23: mol_type = other DNA
tgaaggccttcgagtccctcaag

SEQ ID NO: 36 - DNA, 21 bp, synthetic construct
  source 1..21: mol_type = other DNA
gtgaaggccttcgagtccctc

SEQ ID NO: 37 - DNA, 23 bp, synthetic construct
  source 1..23: mol_type = other DNA
tcgagtccctcaagtccttccag

SEQ ID NO: 38 - DNA, 21 bp, synthetic construct
  source 1..21: mol_type = other DNA
gtcgagtccctcaagtccttc

SEQ ID NO: 39 - DNA, 23 bp, synthetic construct
  source 1..23: mol_type = other DNA
agtccctcaagtccttccagcag

SEQ ID NO: 40 - DNA, 21 bp, synthetic construct
  source 1..21: mol_type = other DNA
gagtccctcaagtccttccag

SEQ ID NO: 41 - DNA, 23 bp, synthetic construct
  source 1..23: mol_type = other DNA
ccctcaagtccttccagcagcag

SEQ ID NO: 42 - DNA, 21 bp, synthetic construct
  source 1..21: mol_type = other DNA
gccctcaagtccttccagcag

SEQ ID NO: 43 - DNA, 23 bp, synthetic construct
  source 1..23: mol_type = other DNA
tcaagtccttccagcagcagcag

SEQ ID NO: 44 - DNA, 21 bp, synthetic construct
  source 1..21: mol_type = other DNA
gtcaagtccttccagcagcag

SEQ ID NO: 45 - DNA, 23 bp, synthetic construct
  source 1..23: mol_type = other DNA
agtccttccagcagcagcagcag

SEQ ID NO: 46 - DNA, 21 bp, synthetic construct
  source 1..21: mol_type = other DNA
gagtccttccagcagcagcag

SEQ ID NO: 47 - DNA, 23 bp, synthetic construct
  source 1..23: mol_type = other DNA
ccttccagcagcagcagcagcag

SEQ ID NO: 48 - DNA, 21 bp, synthetic construct
  source 1..21: mol_type = other DNA
gccttccagcagcagcagcag

SEQ ID NO: 49 - DNA, 23 bp, synthetic construct
  source 1..23: mol_type = other DNA
tccagcagcagcagcagcagcag

SEQ ID NO: 50 - DNA, 21 bp, synthetic construct
  source 1..21: mol_type = other DNA
gtccagcagcagcagcagcag

SEQ ID NO: 51 - DNA, 23 bp, synthetic construct
  source 1..23: mol_type = other DNA
agcagcagcagcagcagcagcag

SEQ ID NO: 52 - DNA, 23 bp, synthetic construct
  source 1..23: mol_type = other DNA
cgccgccgccgccgcctcctcag

SEQ ID NO: 53 - DNA, 23 bp, synthetic construct
  source 1..23: mol_type = other DNA
cgccgcctcctcagcttcctcag

SEQ ID NO: 54 - DNA, 21 bp, synthetic construct
  source 1..21: mol_type = other DNA
gcgccgcctcctcagcttcct

SEQ ID NO: 55 - DNA, 23 bp, synthetic construct
  source 1..23: mol_type = other DNA
tgctgctgctggaaggacttgag

SEQ ID NO: 56 - DNA, 23 bp, synthetic construct
  source 1..23: mol_type = other DNA
gctgctgctgctgctgctggaag

SEQ ID NO: 57 - DNA, 20 bp, synthetic construct
  source 1..20: mol_type = other DNA
gctgctgctgctgctgctgg

SEQ ID NO: 58 - DNA, 23 bp, synthetic construct
  source 1..23: mol_type = other DNA
ggaaggacttgagggactcgaag

SEQ ID NO: 59 - DNA, 20 bp, synthetic construct
  source 1..20: mol_type = other DNA
ggaaggacttgagggactcg

SEQ ID NO: 60 - DNA, 23 bp, synthetic construct
  source 1..23: mol_type = other DNA
cgagtccctcaagtccttccagc

SEQ ID NO: 61 - DNA, 21 bp, synthetic construct
  source 1..21: mol_type = other DNA
gcgagtccctcaagtccttcc

SEQ ID NO: 62 - DNA, 23 bp, synthetic construct
  source 1..23: mol_type = other DNA
gtccctcaagtccttccagcagc

SEQ ID NO: 63 - DNA, 20 bp, synthetic construct
  source 1..20: mol_type = other DNA
gtccctcaagtccttccagc

SEQ ID NO: 64 - DNA, 23 bp, synthetic construct
  source 1..23: mol_type = other DNA
cctcaagtccttccagcagcagc

SEQ ID NO: 65 - DNA, 21 bp, synthetic construct
  source 1..21: mol_type = other DNA
gcctcaagtccttccagcagc

SEQ ID NO: 66 - DNA, 23 bp, synthetic construct
  source 1..23: mol_type = other DNA
caagtccttccagcagcagcagc

SEQ ID NO: 67 - DNA, 21 bp, synthetic construct
  source 1..21: mol_type = other DNA
gcaagtccttccagcagcagc

SEQ ID NO: 68 - DNA, 23 bp, synthetic construct
  source 1..23: mol_type = other DNA
gtccttccagcagcagcagcagc

SEQ ID NO: 69 - DNA, 20 bp, synthetic construct
  source 1..20: mol_type = other DNA
gtccttccagcagcagcagc

SEQ ID NO: 70 - DNA, 23 bp, synthetic construct
  source 1..23: mol_type = other DNA
cttccagcagcagcagcagcagc

SEQ ID NO: 71 - DNA, 21 bp, synthetic construct
  source 1..21: mol_type = other DNA
gcttccagcagcagcagcagc

SEQ ID NO: 72 - DNA, 23 bp, synthetic construct
  source 1..23: mol_type = other DNA
ccagcagcagcagcagcagcagc

SEQ ID NO: 73 - DNA, 21 bp, synthetic construct
  source 1..21: mol_type = other DNA
gccagcagcagcagcagcagc

SEQ ID NO: 74 - DNA, 23 bp, synthetic construct
  source 1..23: mol_type = other DNA
gcagcagcagcagcagcagcagc

SEQ ID NO: 75 - DNA, 23 bp, synthetic construct
  source 1..23: mol_type = other DNA
gctgctgctgctgctgctgctgc

SEQ ID NO: 76 - DNA, 23 bp, synthetic construct
  source 1..23: mol_type = other DNA
gttgctgctgctgctgctgctgc

SEQ ID NO: 77 - DNA, 20 bp, synthetic construct
  source 1..20: mol_type = other DNA
gttgctgctgctgctgctgc

SEQ ID NO: 78 - DNA, 23 bp, synthetic construct
  source 1..23: mol_type = other DNA
gctgttgctgctgctgctgctgc

SEQ ID NO: 79 - DNA, 20 bp, synthetic construct
  source 1..20: mol_type = other DNA
gctgttgctgctgctgctgc

SEQ ID NO: 80 - DNA, 23 bp, synthetic construct
  source 1..23: mol_type = other DNA
gcggctgttgctgctgctgctgc

SEQ ID NO: 81 - DNA, 20 bp, synthetic construct
  source 1..20: mol_type = other DNA
gcggctgttgctgctgctgc

SEQ ID NO: 82 - DNA, 23 bp, synthetic construct
  source 1..23: mol_type = other DNA
gtggcggctgttgctgctgctgc

SEQ ID NO: 83 - DNA, 20 bp, synthetic construct
  source 1..20: mol_type = other DNA
gtggcggctgttgctgctgc

SEQ ID NO: 84 - DNA, 23 bp, synthetic construct
  source 1..23: mol_type = other DNA
gcggtggcggctgttgctgctgc

SEQ ID NO: 85 - DNA, 20 bp, synthetic construct
  source 1..20: mol_type = other DNA
gcggtggcggctgttgctgc

SEQ ID NO: 86 - DNA, 23 bp, synthetic construct
  source 1..23: mol_type = other DNA
gcggcggtggcggctgttgctgc

SEQ ID NO: 87 - DNA, 20 bp, synthetic construct
  source 1..20: mol_type = other DNA
gcggcggtggcggctgttgc

SEQ ID NO: 88 - DNA, 23 bp, synthetic construct
  source 1..23: mol_type = other DNA
gcggcggcggtggcggctgttgc

SEQ ID NO: 89 - DNA, 20 bp, synthetic construct
  source 1..20: mol_type = other DNA
gcggcggcggtggcggctgt

SEQ ID NO: 90 - DNA, 23 bp, synthetic construct
  source 1..23: mol_type = other DNA
cggcggcggcggcggcggtggcg

SEQ ID NO: 91 - DNA, 21 bp, synthetic construct
  source 1..21: mol_type = other DNA
gcggcggcggcggcggcggtg

SEQ ID NO: 92 - DNA, 23 bp, synthetic construct
  source 1..23: mol_type = other DNA
gaggcggcggcggcggcggcggc

SEQ ID NO: 93 - DNA, 20 bp, synthetic construct
  source 1..20: mol_type = other DNA
gaggcggcggcggcggcggc

SEQ ID NO: 94 - DNA, 23 bp, synthetic construct
  source 1..23: mol_type = other DNA
gcagcagcagcagcagcaacagc

SEQ ID NO: 95 - DNA, 20 bp, synthetic construct
  source 1..20: mol_type = other DNA
gcagcagcagcagcagcaac

SEQ ID NO: 96 - DNA, 23 bp, synthetic construct
  source 1..23: mol_type = other DNA
gcagcagcagcagcaacagccgc

SEQ ID NO: 97 - DNA, 20 bp, synthetic construct
  source 1..20: mol_type = other DNA
gcagcagcagcagcaacagc

SEQ ID NO: 98 - DNA, 23 bp, synthetic construct
  source 1..23: mol_type = other DNA
gcagcagcaacagccgccaccgc

SEQ ID NO: 99 - DNA, 20 bp, synthetic construct
  source 1..20: mol_type = other DNA
gcagcagcaacagccgccac

SEQ ID NO: 100 - DNA, 23 bp, synthetic construct
  source 1..23: mol_type = other DNA
gcagcaacagccgccaccgccgc

SEQ ID NO: 101 - DNA, 20 bp, synthetic construct
  source 1..20: mol_type = other DNA
gcagcaacagccgccaccgc

SEQ ID NO: 102 - DNA, 23 bp, synthetic construct
  source 1..23: mol_type = other DNA
gcaacagccgccaccgccgccgc

SEQ ID NO: 103 - DNA, 20 bp, synthetic construct
  source 1..20: mol_type = other DNA
gcaacagccgccaccgccgc

SEQ ID NO: 104 - DNA, 23 bp, synthetic construct
  source 1..23: mol_type = other DNA
acagccgccaccgccgccgccgc

SEQ ID NO: 105 - DNA, 21 bp, synthetic construct
  source 1..21: mol_type = other DNA
gacagccgccaccgccgccgc

SEQ ID NO: 106 - DNA, 23 bp, synthetic construct
  source 1..23: mol_type = other DNA
gcggcggcggcggtggcggctgt

SEQ ID NO: 107 - DNA, 20 bp, synthetic construct
  source 1..20: mol_type = other DNA
gcggcggcggcggtggcggc

SEQ ID NO: 108 - DNA, 23 bp, synthetic construct
  source 1..23: mol_type = other DNA
ctggaaggacttgagggactcga

SEQ ID NO: 109 - DNA, 21 bp, synthetic construct
  source 1..21: mol_type = other DNA
gctggaaggacttgagggact

SEQ ID NO: 110 - DNA, 23 bp, synthetic construct
  source 1..23: mol_type = other DNA
tgctgctggaaggacttgaggga

SEQ ID NO: 111 - DNA, 23 bp, synthetic construct
  source 1..23: mol_type = other DNA
ctgctgctgctggaaggacttga

SEQ ID NO: 112 - DNA, 23 bp, synthetic construct
  source 1..23: mol_type = other DNA
tgctgctgctgctgctggaagga

SEQ ID NO: 113 - DNA, 23 bp, synthetic construct
  source 1..23: mol_type = other DNA
ctgctgctgctgctgctgctgga

SEQ ID NO: 114 - DNA, 21 bp, synthetic construct
  source 1..21: mol_type = other DNA
gctgctgctgctgctgctgct

SEQ ID NO: 115 - DNA, 23 bp, synthetic construct
  source 1..23: mol_type = other DNA
tgctgctgctgctgctgctggaa

SEQ ID NO: 116 - DNA, 21 bp, synthetic construct
  source 1..21: mol_type = other DNA
gtgctgctgctgctgctgctg

SEQ ID NO: 117 - DNA, 23 bp, synthetic construct
  source 1..23: mol_type = other DNA
tggaaggacttgagggactcgaa

SEQ ID NO: 118 - DNA, 21 bp, synthetic construct
  source 1..21: mol_type = other DNA
gtggaaggacttgagggactc

SEQ ID NO: 119 - DNA, 23 bp, synthetic construct
  source 1..23: mol_type = other DNA
gctgctggaaggacttgagggac

SEQ ID NO: 120 - DNA, 20 bp, synthetic construct
  source 1..20: mol_type = other DNA
gctgctggaaggacttgagg

SEQ ID NO: 121 - DNA, 23 bp, synthetic construct
  source 1..23: mol_type = other DNA
gctgctgctgctgctggaaggac

SEQ ID NO: 122 - DNA, 20 bp, synthetic construct
  source 1..20: mol_type = other DNA
gctgctgctgctgctggaag

SEQ ID NO: 123 - DNA, 23 bp, synthetic construct
  source 1..23: mol_type = other DNA
gctggaaggacttgagggactcg

SEQ ID NO: 124 - DNA, 20 bp, synthetic construct
  source 1..20: mol_type = other DNA
gctggaaggacttgagggac

SEQ ID NO: 125 - DNA, 23 bp, synthetic construct
  source 1..23: mol_type = other DNA
tgctggaaggacttgagggactc

SEQ ID NO: 126 - DNA, 21 bp, synthetic construct
  source 1..21: mol_type = other DNA
gtgctggaaggacttgaggga

SEQ ID NO: 127 - DNA, 23 bp, synthetic construct
  source 1..23: mol_type = other DNA
ctgctggaaggacttgagggact

SEQ ID NO: 128 - DNA, 21 bp, synthetic construct
  source 1..21: mol_type = other DNA
gctgctggaaggacttgaggg

SEQ ID NO: 129 - DNA, 23 bp, synthetic construct
  source 1..23: mol_type = other DNA
gctgctgctgctggaaggacttg

SEQ ID NO: 130 - DNA, 20 bp, synthetic construct
  source 1..20: mol_type = other DNA
gctgctgctgctggaaggac

SEQ ID NO: 131 - DNA, 23 bp, synthetic construct
  source 1..23: mol_type = other DNA
tgctgctgctgctggaaggactt

SEQ ID NO: 132 - DNA, 21 bp, synthetic construct
  source 1..21: mol_type = other DNA
gtgctgctgctgctggaagga

SEQ ID NO: 133 - DNA, 23 bp, synthetic construct
  source 1..23: mol_type = other DNA
ctgctgctgctgctggaaggact

SEQ ID NO: 134 - DNA, 21 bp, synthetic construct
  source 1..21: mol_type = other DNA
gctgctgctgctgctggaagg

SEQ ID NO: 135 - DNA, 27 bp, synthetic construct
  source 1..27: mol_type = other DNA
cttcgagtccctcaagtccttccagca

SEQ ID NO: 136 - DNA, 23 bp, synthetic construct
  source 1..23: mol_type = other DNA
gagtccctcaagtccttccagca

SEQ ID NO: 137 - DNA, 27 bp, synthetic construct
  source 1..27: mol_type = other DNA
ttcgagtccctcaagtccttccagcag

SEQ ID NO: 138 - DNA, 27 bp, synthetic construct
  source 1..27: mol_type = other DNA
tccctcaagtccttccagcagcagcag

SEQ ID NO: 139 - DNA, 27 bp, synthetic construct
  source 1..27: mol_type = other DNA
tccttccagcagcagcagcagcagcag

SEQ ID NO: 140 - DNA, 27 bp, synthetic construct
  source 1..27: mol_type = other DNA
cttccagcagcagcagcagcagcagca

SEQ ID NO: 141 - DNA, 23 bp, synthetic construct
  source 1..23: mol_type = other DNA
cagcagcagcagcagcagcagca

SEQ ID NO: 142 - DNA, 27 bp, synthetic construct
  source 1..27: mol_type = other DNA
ttccagcagcagcagcagcagcagcag

SEQ ID NO: 143 - DNA, 27 bp, synthetic construct
  source 1..27: mol_type = other DNA
gttgctgctgctgctgctgctgctgct

SEQ ID NO: 144 - DNA, 23 bp, synthetic construct
  source 1..23: mol_type = other DNA
ctgctgctgctgctgctgctgct

SEQ ID NO: 145 - DNA, 27 bp, synthetic construct
  source 1..27: mol_type = other DNA
cttgagggactcgaaggccttcatcag

SEQ ID NO: 146 - DNA, 23 bp, synthetic construct
  source 1..23: mol_type = other DNA
agggactcgaaggccttcatcag

SEQ ID NO: 147 - DNA, 27 bp, synthetic construct
  source 1..27: mol_type = other DNA
cttcctcagccgccgccgcaggcacag

SEQ ID NO: 148 - DNA, 23 bp, synthetic construct
  source 1..23: mol_type = other DNA
ctcagccgccgccgcaggcacag

SEQ ID NO: 149 - DNA, 27 bp, synthetic construct
  source 1..27: mol_type = other DNA
tcctcagccgccgccgcaggcacagcc

SEQ ID NO: 150 - DNA, 23 bp, synthetic construct
  source 1..23: mol_type = other DNA
cagccgccgccgcaggcacagcc

SEQ ID NO: 151 - DNA, 19 bp, synthetic construct
  source 1..19: mol_type = other DNA
gggagaccgccatggcgac

SEQ ID NO: 152 - DNA, 20 bp, synthetic construct
  source 1..20: mol_type = other DNA
ggctgaggcagcagcggctg

SEQ ID NO: 153 - DNA, 25 bp, synthetic construct
  source 1..25: mol_type = other DNA
catggcgaccctggaaaagctgatg

SEQ ID NO: 154 - DNA, 21 bp, synthetic construct
  source 1..21: mol_type = other DNA
ctgaggaagctgaggaggcgg

SEQ ID NO: 155 - DNA, 15 bp, synthetic construct
  source 1..15: mol_type = other DNA
cagcagcagcagcag

SEQ ID NO: 156 - DNA, 12 bp, synthetic construct
  source 1..12: mol_type = other DNA
cagcagcagcag

SEQ ID NO: 157 - DNA, 57 bp, Homo sapiens
  source 1..57: mol_type = genomic DNA
aaggccttcgagtccctcaagtccttccagcagcagcagcagcagcagcagcagcag

SEQ ID NO: 158 - protein, 19 aa, Homo sapiens
KAFESLKSFQQQQQQQQQQ

SEQ ID NO: 159 - DNA, 18 bp, synthetic construct
  source 1..18: mol_type = other DNA
cagcagcagcagcagcag

SEQ ID NO: 160 - DNA, 14 bp, synthetic construct
  source 1..14: mol_type = other DNA
agcagcagcagcag

SEQ ID NO: 161 - DNA, 18 bp, synthetic construct
  source 1..18: mol_type = other DNA
ctgctgctgctgctgctg

SEQ ID NO: 162 - DNA, 24 bp, synthetic construct
  source 1..24: mol_type = other DNA
ctgctgctgctgctgctgctgctg

SEQ ID NO: 163 - DNA, 139 bp, Homo sapiens
  source 1..139: mol_type = genomic DNA
gaaggccttcgagtccctcaagtccttccagcagcagcagcagcagcagcagcagcagcagcagcagcagcagcagcagcaacagccgccaccgccgccgccgccgccgccgcctcctcagcttcctcagccgccgccg

SEQ ID NO: 164 - protein, 46 aa, Homo sapiens
KAFESLKSFQQQQQQQQQQQQQQQQQQQPPPPPPPPPPPQLPQPPP

SEQ ID NO: 165 - DNA, 136 bp, Homo sapiens
  source 1..136: mol_type = genomic DNA
ggccttcgagtccctcaagtccttccagcagcagcagcagcagcagcagcagcagcagcagcagcagcagcagcagcaacagccgccaccgccgccgccgccgccgccgcctcctcagcttcctcagccgccgccg

SEQ ID NO: 166 - protein, 45 aa, Homo sapiens
AFESLKSFQQQQQQQQQQQQQQQQQQQPPPPPPPPPPPQLPQPPP

SEQ ID NO: 167 - DNA, 137 bp, Homo sapiens
  source 1..137: mol_type = genomic DNA
aggccttcgagtccctcaagtccttccagcagcagcagcagcagcagcagcagcagcagcagcagcagcagcagcagcaacagccgccaccgccgccgccgccgccgccgcctcctcagcttcctcagccgccgccg

SEQ ID NO: 168 - DNA, 136 bp, Homo sapiens
  source 1..136: mol_type = genomic DNA
catggcgaccctggaaaagctgatgaaggccttcgagtccctcaagtccttccagcagcagcagcagcagcagcagcagcagcagcagcagcagcagcagcagcaacagccgccaccgccgccgccgccgccgccg

SEQ ID NO: 169 - protein, 45 aa, Homo sapiens
MATLEKLMKAFESLKSFQQQQQQQQQQQQQQQQQQQPPPPPPPPP

SEQ ID NO: 170 - DNA, 166 bp, Homo sapiens
  source 1..166: mol_type = genomic DNA
catggcgaccctggaaaagctgatgaaggccttcgagtccctcaagtccttccagcagcagcagcagcagcagcagcagcagcagcagcagcagcagcagcagcaacagccgccaccgccgccgccgccgccgccgcctcctcagcttcctcagccgccgccgcag

SEQ ID NO: 171 - protein, 55 aa, Homo sapiens
MATLEKLMKAFESLKSFQQQQQQQQQQQQQQQQQQQPPPPPPPPPPPQLPQPPPQ

SEQ ID NO: 172 - DNA, 133 bp, Homo sapiens
  source 1..133: mol_type = genomic DNA
cgagtccctcaagtccttccagcagcagcagcagcagcagcagcagcagcagcagcagcagcagcagcagcaacagccgccaccgccgccgccgccgccgccgcctcctcagcttcctcagccgccgccgcag

SEQ ID NO: 173 - protein, 44 aa, Homo sapiens
ESLKSFQQQQQQQQQQQQQQQQQQQPPPPPPPPPPPQLPQPPPQ

SEQ ID NO: 174 - DNA, 20 bp, synthetic construct
  source 1..20: mol_type = other DNA
gctgctgctgctgctgctgc

SEQ ID NO: 175 - DNA, 23 bp, synthetic construct
  source 1..23: mol_type = other DNA
agtccctcaagtccttccagcag

SEQ ID NO: 176 - DNA, 23 bp, synthetic construct
  source 1..23: mol_type = other DNA
tcaagtccttccagcagcagcag

SEQ ID NO: 177 - DNA, 23 bp, synthetic construct
  source 1..23: mol_type = other DNA
tccagcagcagcagcagcagcag

SEQ ID NO: 178 - DNA, 23 bp, synthetic construct
  source 1..23: mol_type = other DNA
agcagcagcagcagcagcagcag